SEQUENCE LISTING (56 sequences)

SEQ ID NO: 1 - protein, 452 aa, Unknown
  Full-length Phenylalanine Hydroxylase (PAH)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 2 - protein, 338 aa, Artificial sequence
  N-terminal phenylalanine Hydroxylase (PAH) fragment C237/N-terminal Nostoc punctiforme (Npu) split DnaE intein
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 3 - protein, 252 aa, Artificial sequence
  C-terminal Nostoc punctiforme (Npu) split DnaE intein/C-terminal phenylalanine Hydroxylase (PAH) fragment C237
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 4 - protein, 366 aa, Artificial sequence
  N-terminal phenylalanine Hydroxylase (PAH) fragment C265/N-terminal Nostoc punctiforme (Npu) split DnaE intein
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 5 - protein, 224 aa, Artificial sequence
  C-terminal Nostoc punctiforme (Npu) split DnaE intein/C-terminal phenylalanine Hydroxylase (PAH) fragment C265
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 6 - protein, 385 aa, Artificial sequence
  N-terminal phenylalanine Hydroxylase (PAH) fragment C284/N-terminal Nostoc punctiforme (Npu) split DnaE intein
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 7 - protein, 205 aa, Artificial sequence
  C-terminal Nostoc punctiforme (Npu) split DnaE intein/C-terminal phenylalanine Hydroxylase (PAH) fragment C284
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 8 - protein, 435 aa, Artificial sequence
  N-terminal phenylalanine Hydroxylase (PAH) fragment C334/N-terminal Nostoc punctiforme (Npu) split DnaE intein
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 9 - protein, 155 aa, Artificial sequence
  C-terminal Nostoc punctiforme (Npu) split DnaE intein/C-terminal phenylalanine Hydroxylase (PAH) fragment C334
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 10 - protein, 250 aa, Unknown
  GTP cyclohydrolase I
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 11 - protein, 22 aa, Unknown
  2A self-cleaving peptide
GSGATNFSLLKQAGDVEENPGP

SEQ ID NO: 12 - protein, 724 aa, Artificial sequence
  Full-length phenylalanine Hydroxylase (PAH)-P2A-(GTP-CH1)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 13 - protein, 610 aa, Artificial sequence
  N-terminal phenylalanine Hydroxylase (PAH) fragment C237/N-terminal Nostoc punctiforme (Npu) split DnaE intein-P2A-(GTP-CH1)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 14 - protein, 524 aa, Artificial sequence
  C-terminal Nostoc punctiforme (Npu) split DnaE intein/C-terminal phenylalanine Hydroxylase (PAH) fragment C237-P2A-(GTP-CH1)
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 15 - protein, 638 aa, Artificial sequence
  N-terminal phenylalanine Hydroxylase (PAH) fragment C265/N-terminal Nostoc punctiforme (Npu) split DnaE intein-P2A-(GTP-CH1)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 16 - protein, 496 aa, Artificial sequence
  C-terminal Nostoc punctiforme (Npu) split DnaE intein/C-terminal phenylalanine Hydroxylase (PAH) fragment C265-P2A-(GTP-CH1)
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 17 - protein, 657 aa, Artificial sequence
  N-terminal phenylalanine Hydroxylase (PAH) fragment C284/N-terminal Nostoc punctiforme (Npu) split DnaE intein-P2A-(GTP-CH1)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 18 - protein, 477 aa, Artificial sequence
  C-terminal Nostoc punctiforme (Npu) split DnaE intein/C-terminal phenylalanine Hydroxylase (PAH) fragment C284-P2A-(GTP-CH1)
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 19 - protein, 707 aa, Artificial sequence
  N-terminal phenylalanine Hydroxylase (PAH) fragment C334/N-terminal Nostoc punctiforme (Npu) split DnaE intein-P2A-(GTP-CH1)
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 20 - protein, 427 aa, Artificial sequence
  C-terminal Nostoc punctiforme (Npu) split DnaE intein/C-terminal phenylalanine Hydroxylase (PAH) fragment C334-P2A-(GTP-CH1)
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 21 - protein, 246 aa, Artificial sequence
  synthetic sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYSDLELK

SEQ ID NO: 22 - protein, 236 aa, Artificial sequence
  synthetic sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 23 - protein, 373 aa, Unknown
  Full-length glutamine synthetase (GS)
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 24 - protein, 154 aa, Artificial sequence
  N-terminal GS fragment C53/N-terminal Nostoc punctiforme (Npu) split DnaE intein
MTTSASSHLNKGIKQVYMSLPQGEKVQAMYIWIDGTGEGLRCKTRTLDSEPKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 25 - protein, 357 aa, Artificial sequence
  C-terminal Nostoc punctiforme (Npu) split DnaE intein/C-terminal GS fragment C53
MIKIATRKYLGKQNVYDIGVERDHNFALKNGFIASNCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 26 - protein, 218 aa, Artificial sequence
  N-terminal GS fragment C117/N-terminal Nostoc punctiforme (Npu) split DnaE intein
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 27 - protein, 293 aa, Artificial sequence
  C-terminal Nostoc punctiforme (Npu) split DnaE intein/C-terminal GS fragment C117
MIKIATRKYLGKQNVYDIGVERDHNFALKNGFIASNCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 28 - protein, 284 aa, Artificial sequence
  N-terminal GS fragment C183/N-terminal Nostoc punctiforme (Npu) split DnaE intein
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 29 - protein, 227 aa, Artificial sequence
  C-terminal Nostoc punctiforme (Npu) split DnaE intein/C-terminal GS fragment C183
MIKIATRKYLGKQNVYDIGVERDHNFALKNGFIASNCLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 30 - protein, 330 aa, Artificial sequence
  N-terminal GS fragment C229/N-terminal Nostoc punctiforme (Npu) split DnaE intein
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 31 - protein, 181 aa, Artificial sequence
  C-terminal Nostoc punctiforme (Npu) split DnaE intein/C-terminal GS fragment C229
MIKIATRKYLGKQNVYDIGVERDHNFALKNGFIASNCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 32 - protein, 353 aa, Artificial sequence
  N-terminal GS fragment C252/N-terminal Nostoc punctiforme (Npu) split DnaE intein
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 33 - protein, 158 aa, Artificial sequence
  C-terminal Nostoc punctiforme (Npu) split DnaE intein/C-terminal GS fragment C252
MIKIATRKYLGKQNVYDIGVERDHNFALKNGFIASNCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 34 - protein, 279 aa, Unknown
  Full-length Thymidylate Synthase (TYMS)
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 35 - protein, 144 aa, Artificial sequence
  N-terminal TYMS fragment C41/N-terminal Nostoc punctiforme (Npu) split DnaE intein
MPVAGSELPRRPLPPAAQERDAEPRPPHGELQYLGQIQHILRCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 36 - protein, 273 aa, Artificial sequence
  C-terminal Nostoc punctiforme (Npu) split DnaE intein/C-terminal TYMS fragment C41
MIKIATRKYLGKQNVYDIGVERDHNFALKNGFIASNCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 37 - protein, 262 aa, Artificial sequence
  N-terminal TYMS fragment C161/N-terminal Nostoc punctiforme (Npu) split DnaE intein
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 38 - protein, 155 aa, Artificial sequence
  C-terminal Nostoc punctiforme (Npu) split DnaE intein/C-terminal TYMS fragment C161
MIKIATRKYLGKQNVYDIGVERDHNFALKNGFIASNCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 39 - protein, 266 aa, Artificial sequence
  N-terminal TYMS fragment C165/N-terminal Nostoc punctiforme (Npu) split DnaE intein
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 40 - protein, 151 aa, Artificial sequence
  C-terminal Nostoc punctiforme (Npu) split DnaE intein/C-terminal TYMS fragment C165
MIKIATRKYLGKQNVYDIGVERDHNFALKNGFIASNCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 41 - protein, 277 aa, Artificial sequence
  N-terminal TYMS fragment C176/N-terminal Nostoc punctiforme (Npu) split DnaE intein
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 42 - protein, 140 aa, Artificial sequence
  C-terminal Nostoc punctiforme (Npu) split DnaE intein/C-terminal TYMS fragment C176
MIKIATRKYLGKQNVYDIGVERDHNFALKNGFIASNCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 43 - DNA, 546 bp, Artificial sequence
  EF1alpha mutated promoter (attenuated)
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 44 - DNA, 546 bp, Unknown
  EF1a wild-type promoter
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 45 - DNA, 350 bp, Artificial sequence
  synthetic sequence
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 46 - DNA, 588 bp, Artificial sequence
  synthetic sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 47 - DNA, 6397 bp, Artificial sequence
  synthetic sequence
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgt

SEQ ID NO: 48 - DNA, 10347 bp, Artificial sequence
  synthetic sequence
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcc

SEQ ID NO: 49 - DNA, 13923 bp, Artificial sequence
  synthetic sequence
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 50 - DNA, 613 bp, Artificial sequence
  synthetic sequence
gaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactga

SEQ ID NO: 51 - DNA, 223 bp, Artificial sequence
  synthetic sequence
atgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaatt

SEQ ID NO: 52 - DNA, 2005 bp, Artificial sequence
  synthetic sequence
gcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaa

SEQ ID NO: 53 - protein, 102 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 54 - protein, 36 aa, Nostoc punctiforme
MIKIATRKYLGKQNVYDIGVERDHNFALKNGFIASN

SEQ ID NO: 55 - protein, 11 aa, Artificial sequence
  synthetic sequence
  VARIANT (1)..(7): Xaa is any naturally occuring amino acid
XXXXXXXCLSY

SEQ ID NO: 56 - protein, 11 aa, Artificial sequence
  synthetic sequence
  VARIANT (6)..(11): Xaa is any naturally occuring amino acid
IASNCXXXXXX

(19) United States
(12) Patent Application Publication
(10) Pub. No.: US 2024/0124850 A1
(43) Pub. Date: Apr. 18, 2024

(54) Auxotrophic Cells for Virus Production and Compositions and Methods of Making

(71) Applicant: Shape Therapeutics Inc., Seattle, WA (US)

(72) Inventors: Kenneth Prentice, Seattle, WA (US); Lindsey Nicole Deis Huffman, Lynwood, WA (US); Sandhya Pande, Kent, WA (US)

(21) Appl. No.: 18/277,033

(22) PCT Filed: Mar. 3, 2022
(86) PCT No.: PCT/US2022/018784

§ 371 Date: Aug. 11, 2023

Related U.S. Application Data
(60) Provisional application No. 63/156,203, filed on Mar. 3, 2021.

(51) Int. Cl. C12N 7/00; C07K 14/005; C12N 5/00; C12N 5/071; C12N 9/00; C12N 9/06; C12N 9/10; C12N 15/52

(52) U.S. Cl. CPC C12N 7/00; C07K 14/005; C12N 5/0018; C12N 5/0682; C12N 5/0686; C12N 9/0016; C12N 9/1007; C12N 9/93; C12N 15/52; C12N 2500/32; C12N 2501/999; C12N 2750/14122; C12N 2750/14151; C12Y 114/16001; C12Y 201/01045; C12Y 603/01002

(57) ABSTRACT

Disclosed herein are cells and cell lines that are selected for retention of at least two exogenous nucleic acid constructs using a single selective pressure. Also disclosed herein are compositions and methods for generating recombinant cells and cell lines using a single selective pressure.

### CROSS-REFERENCE TO RELATED APPLICATIONS

This application claims priority benefit of U.S. Provisional Application No. 63/156,203, filed Mar. 3, 2021, the disclosure of which is incorporated herein by reference in its entirety.

### INCORPORATION BY REFERENCE OF SEQUENCE LISTING PROVIDED AS A TEXT FILE

A Sequence Listing is provided herewith as a text file, “SHPE-002WO Seq List_ST25.txt,” created on Mar. 1, 2022 and having a size of 171,000 bytes. The contents of the text file are incorporated by reference herein in their entirety.

### BACKGROUND

Numerous biotechnology applications require the introduction of exogenous nucleic acid into a host cell. A critical step in generating host cells that contain exogenous nucleic acid is the process of selecting the cells that retain the exogenous nucleic acid of interest. It is important to be able to efficiently select host cells that have retained one or more exogenous nucleic acids of interest.

Antibiotic resistance genes are frequently used for selecting host cells that retain exogenous nucleic acid. For example, the exogenous nucleic acid introduced to the host cell may encode a protein that confers resistance to a particular antibiotic. Host cells can then be selected for retention of the exogenous nucleic acid by subjecting the cells to media containing the antibiotic. Only cells which have retained the exogenous nucleic acid and, accordingly, have acquired the ability to grow in the presence of the antibiotic, will remain viable under the selection conditions. While effective, this method is often undesirable due to the use of antibiotics and the potential risk of propagating resistance genes. Further, it is generally undesirable to subject cells to multiple selective pressures in order to introduce two or more nucleic acid constructs into a host cell or cell line.

Accordingly, there is a need for improved compositions and methods for generation and selection of cells that have retained exogenous nucleic acid of interest. In particular, there is need for improved methods of generating and selecting cells that have retained two or more nucleic acid constructs.

### SUMMARY

In some embodiments, provided herein is a method of generating a recombinant host cell that includes a first and second exogenous nucleic acid construct and selecting for the eukaryotic host cell that includes both exogenous nucleic acid constructs with a single selective pressure. In some embodiments, the method comprises introducing into a host cell (a) first exogenous nucleic acid construct comprising a first polynucleotide of interest and a first portion of a selectable marker and (b) a second exogenous nucleic acid construct comprising a second polynucleotide of interest and a second portion of a selectable marker. In some embodiments, the first portion of the selectable marker encodes a nonfunctional first portion of a selectable protein and the second portion of the selectable marker encodes a nonfunctional second portion of the selectable protein. In some embodiments, the nonfunctional first and second portions of the selectable protein are capable of assembling in the cell to create a functional selectable protein.

In some embodiments, the host cell is a eukaryotic cell, e.g., a mammalian cell. In some embodiments, the mammalian cell is a human embryonic kidney (HEK) cell, chinese hamster ovary (CHO) cell, HeLa cell, or a derivative thereof. In some embodiments, the HEK cell is an HEK293 cell.

In some embodiments, the host cell is suspension-adapted. In some embodiments, the recombinant eukaryotic host cell is capable of virus production. In some embodiments, the host cell is a viral production cell.

In some embodiments, the first exogenous nucleic acid construct, the second exogenous nucleic acid construct, or both the first and second exogenous nucleic acid constructs become stably incorporated in the host cell genome. In another aspect, plasmids or episomes are provided comprising the nucleic acid constructs as disclosed herein. In some embodiments, the plasmids or episomes further comprise Epstein-Barr virus (EBV) sequences to stably maintain the constructs extrachromosomally. In some embodiments, the first polynucleotide of interest encodes an adeno-associated virus (AAV) Rep protein, an AAV Cap protein, an adenoviral helper protein, a first payload, or any combination thereof. In some embodiments, the second polynucleotide of interest encodes an adeno-associated virus (AAV) Rep protein, an AAV Cap protein, an adenoviral helper protein, a second payload, or any combination thereof.

In some embodiments, the first and/or second payload is a guide RNA, a tRNA, or a gene (e.g., a transgene). In some embodiments, the first and/or second payload is a nucleic acid sequence that encodes a protein. In some embodiments, the first and/or second payload comprises a gene for replacement gene therapy. In some embodiments, the first and/or second payload comprises a homology construct for homologous recombination.

In some embodiments, the selectable marker does not confer resistance to an antibiotic or a toxin. In some embodiments, wherein the single selective pressure is not an antibiotic or a toxin. In some embodiments, the selectable protein is a functional enzyme. In some embodiments, the functional enzyme is not endogenous to the host cell. In some embodiments, the function enzyme is endogenous to the host cell.

In some embodiments, the functional enzyme catalyzes a reaction that results in the production of a molecule necessary for growth of the host cell when the host cell is grown in media deficient for the molecule. In some embodiments, the functional enzyme catalyzes the conversion of an amino acid into the molecule necessary for growth of the host cell. In some embodiments the enzyme is dihydrofolate reductase (DHFR), glutamine synthetase (GS), thymidylate synthase (TYMS), phenylalanine hydroxylase (PAH), or any combination thereof.

In some embodiments, the molecule necessary for growth of the host cell is hypoxanthine, glutamine, tyrosine, and/or thymidine.

In some embodiments, PAH catalyzes the conversion of phenylalanine to tyrosine in the presence of (6R)-5,6,7,8-tetrahydrobiopterin (BH4) or a BH4 precursor. In some embodiments, the BH4 precursor is 7,8-dihydrobiopterin (7,8-BH2).

In some embodiments, the host cell is grown in a media deficient for a molecule necessary for growth of the host cell. In some embodiments, the molecule necessary for growth of the host cell is tyrosine.

In some embodiments, the first portion of the selectable marker is fused to a coding sequence of an N-terminal fragment of a split intein. In some embodiments, the second portion of the selectable marker is fused to a coding sequence of a C-terminal fragment of a split intein. In some embodiments, the split intein is derived from the Nostoc punctiforme (Npu) DnaE intein, the Synechocystis species, strain PCC6803 (Ssp) DnaE intein, or the consensus DnaE intein (Cfa).

In some embodiments, the nonfunctional first portion of a selectable protein and the nonfunctional second portion of a selectable protein, once joined to generate the functional selectable protein, are linked by a peptide bond at a split point in the functional selectable protein. In some embodiments, the split point is a cysteine or serine residue within the catalytic domain of the functional selectable protein. In some embodiments, the nonfunctional first portion of a selectable protein is an N-terminal fragment of the functional selectable protein. In some embodiments, the nonfunctional second portion of a selectable protein is a C-terminal fragment of the functional selectable protein. In some embodiments, the N-terminal residue of the nonfunctional second portion of a selectable protein is cysteine or serine.

In some embodiments, the functional selectable protein is a functional enzyme. In some embodiments, the functional enzyme is required for production of a molecule required for cell growth. In some embodiments, the functional enzyme is glutamine synthetase (GS), thymidylate synthase (TYMS), or phenylalanine hydroxylase (PAH). In some embodiments, the polypeptide is an enzyme that catalyzes production of a cofactor.

In some embodiments, the first or second exogenous nucleic acid construct further encodes a helper enzyme, wherein expression of the helper enzyme facilitates growth of the host cell in conjunction with the functional enzyme upon application of the single selective pressure. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the first or second exogenous nucleic acid construct may increase helper enzyme levels thereby increasing production of the molecule required for cell growth, by, e.g., increasing levels of a co-factor required for enzyme activity. In some embodiments, the first or the second exogenous nucleic acid construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the GTP-CH1 produces the cofactor (6R)-5,6,7,8-tetrahydrobiopterin (BH4) that is required for conversion of phenylalanine to tyrosine. In some embodiments, the host cell is a cell that expresses or is genetically modified to express GTP-CH1. In some embodiments, expression of GTP-CH1 facilitates growth of the host cell in conjunction with functional PAH upon application of the single selective pressure.

In some embodiments, the functional enzyme is PAH and the host cell is grown in media comprising a cofactor and deficient in tyrosine. In some embodiments, the cofactor is (6R)-5,6,7,8-tetrahydrobiopterin (BH4). In some embodiments, the cofactor is a (6R)-5,6,7,8-tetrahydrobiopterin (BH4) precursor molecule. In some embodiments, the BH4 precursor molecule is 7,8-dihydobiopterin (7,8-BH2).

In some embodiments, the method further comprises applying the single selective pressure. In some embodiments, the single selective pressure comprises growing the host cell in media deficient in at least one nutrient. In some embodiments, the host cell is grown in media deficient in tyrosine and cells expressing functional PAH are selected.

In some embodiments, the method further comprises applying a second selective pressure, wherein application of the second selective pressure selects for cells that highly express the first portion and the second portion of the selectable marker. In some embodiments, the second selective pressure is the presence of an inhibitor. In some embodiments, the inhibitor inhibits activity of the functional enzyme.

In some embodiments, a virus particle produced by the recombinant eukaryotic host cell has an increased safety profile as compared to a virus particle produced by a method wherein the single selective pressure is an antibiotic.

In some embodiments, the method yields an increase in a number of clones integrated with the first and second polynucleotide of interest as compared to a method wherein the single selective pressure is an antibiotic or a method wherein two different selectable markers are used.

In another aspect, provided herein is a composition of plasmids for stably transfecting a eukaryotic host cell with two or more exogenous nucleic acid constructs that are capable of being retained in the cell with a single selective pressure. In some embodiments, the composition comprises (a) a first plasmid comprising a first polynucleotide of interest and a first portion of a selectable marker and (b) a second plasmid comprising a second polynucleotide of interest and a second portion of a selectable marker. In another aspect, episomes are provided comprising the constructs as disclosed herein. In some embodiments, the plasmids or episomes further comprise Epstein-Barr virus (EBV) sequences to stably maintain the constructs extrachromosomally.

In another aspect, provided herein is a cell or cell line selected to retain a first and second exogenous nucleic acid construct with a single selective pressure. In some embodiments, the first exogenous nucleic acid construct comprises a first polynucleotide of interest and a first portion of a selectable marker and the second exogenous nucleic acid construct comprises a second polynucleotide of interest and a second portion of a selectable marker. In some embodiments, the first portion of the selectable marker encodes a nonfunctional first portion of a selectable protein and the second portion of the selectable marker encodes a nonfunctional second portion of a selectable protein. In some embodiments, survival of the cell or cell line under the single selective pressure requires expression of a functional selectable protein and the functional selectable protein is generated by protein trans-splicing the nonfunctional first and second portions of the selectable protein.

In another aspect, provided herein is a method of selecting a cell for retention of at least two exogenous nucleic acid constructs. In some embodiments, a single selective pressure is used for selecting a cell for retention of at least two nucleic acid constructs. In some embodiments, expression of a functional selectable protein is required for the cell to survive the selective pressure. In some embodiments, the functional selectable protein is expressed following protein trans-splicing of nonfunctional polypeptide fragments where the nonfunctional polypeptide fragments are encoded by at least two separate nucleic acid constructs.

### BRIEF DESCRIPTION OF THE DRAWINGS

These and other features, aspects, and advantages of the present invention will become better understood with regard to the following description, and accompanying drawings, where:

FIGS. 1A-1C provide a schematic overview of the split selectable marker system. FIG. 1A depicts the selectable marker split into an N-terminal fragment and a C-terminal fragment. FIG. 1B depicts two plasmids that separately comprise a polynucleotide of interest (Transgene 1 or 2) and nucleic acid encoding either the N-terminal or C-terminal fragment of a selectable marker protein. FIG. 1C depicts a cell expressing the full-length selectable marker protein and both transgenes.

FIG. 2 is a schematic depicting the criterion for identifying a split point for a selectable marker protein engineered for trans protein splicing via the split NpuDnaE intein. Partial sequence of a fusion protein comprising an N-terminal fragment of a functional selectable protein (e.g., PAH) and an N-terminal fragment of NpuDnaE intein is set forth in SEQ ID NO:55. Partial sequence of a fusion protein comprising a C-terminal fragment of the NpuDnaE intein and a C-terminal fragment of the functional selectable protein (e.g., PAH) is set forth in SEQ ID NO:56.

FIG. 3 is a cartoon representation of the protein structure of phenylalanine hydroxylase (PAH) displayed in two different perspectives to show the position of each of the four cysteine residues identified as a potential split point for a split intein. For example, the PAH protein can be split at Cys237, Cys265, Cys284, or Cys334.

FIGS. 4A-4B are schematics depicting plasmids encoding the N-terminal PAH fragment/N-terminal NpuDnaE intein (PAH N-term) (FIG. 4A) and the C-terminal NpuDnaE intein/C-terminal PAH fragment (PAH C-term) (FIG. 4B). The plasmids encoding the N- and C-terminal portions of the PAH selectable protein were generated by separately introducing the split point at each of residues Cys237, Cys265, Cys284, and Cys334. Promoters shown in FIG. 4 (e.g., CMV and EF-1 alpha) can be swapped out for any known promoter. Examples of promoters include, but are not limited to the following: CMV, EF-1 alpha, UBC, PGK, CAGG, SV40, and TRE.

FIGS. 5A-5B show the head-to-head vector configuration for co-expression of a gene of interest with the PAH gene. FIG. 5A shows the configuration for the plasmid encoding full-length PAH. FIG. 5B shows the configuration for the plasmids encoding each of the split intein/PAH fragments. Promoters shown in FIGS. 5A-5B (e.g., CMV and EF-1 alpha) can be swapped out for any known promoter. Examples of promoters include, but are not limited to the following: CMV, EF-1 alpha, UBC, PGK, CAGG, SV40, and TRE.

FIGS. 6A-6D show viability of cells co-transfected with plasmids encoding N-terminal PAH fragment/N-terminal NpuDnaE intein (PAH N-term) and C-terminal NpuDnaE intein/C-terminal PAH fragment (PAH C-term) where the split point was located at Cys237 (FIG. 6A), Cys265 (FIG. 6B), Cys284 (FIG. 6C), or Cys334 (FIG. 6D) of PAH, following selection in tyrosine-deficient media containing (6R)-5,6,7,8-tetrahydrobiopterin (BH4).

FIGS. 7A-7B show cell viability of cells transfected with full-length PAH (FIG. 7A) or split intein PAH (FIG. 7B), following selection in tyrosine-deficient media containing 7,8-dihydrobiopterin (7,8-BH2). FIG. 7C shows cells transfected with split intein PAH and then cultured in selection media comprising 7,8-BH2 had increased viability and viable cell density after four days in selection media compared to cells cultured in selection media comprising BH4.

FIGS. 8A-8B show vector diagrams of PAH selection cassettes for co-expression with GTP cyclohydrolase (GTP-CH1). The PAH and GTP-CH1 are expressed under the control of a single promoter (FIG. 8A) or under the control of separate promoters from separate expression cassettes (FIG. 8B). In FIG. 8A, the GTP-CH1 and PAH are produced either after cleavage of the P2A cleavable peptide or using an IRES. Promoters shown in FIGS. 8A and 8B (e.g., CMV, EF-1 alpha, and CAGG) can be swapped out for any known promoter. Examples of promoters include, but are not limited to the following: CMV, EF-1 alpha, UBC, PGK, CAGG, bGH, SV40, and TRE.

FIG. 9 shows the viability of cells transfected with GTP-CH1 following full-length PAH and a cleavable P2A peptide. Similar viability data are observed when GTP-CH1 and PAH are expressed in separate expression cassettes.

FIGS. 10A-10B show vector diagrams of PAH co-expressed with GTP cyclohydrolase (GTP-CH1) adjacent to the gene of interest (GOI). FIG. 10A shows the configuration for full-length PAH in conjunction with GTP-CH1-(IRES/P2A)-GOI. FIG. 10B shows the configuration for the plasmids encoding each of the split intein/PAH fragments in conjunction with GTP-CH1-(IRES/P2A)-GOI. Promoters shown in FIG. 10 (e.g., CMV and EF-1 alpha) can be swapped out for any known promoters. Examples of promoters include, but are not limited to the following: CMV, EF-1 alpha, UBC, PGK, CAGG, SV40, and TRE.

FIGS. 11A-11B show the growth of cells transfected with plasmid containing either (GTP-CH1)-IRES-GOI with the N-terminal PAH fragment/N-terminal NpuDnaE intein (PAH N-term(G)); (GTP-CH1)-IRES-GOI with the C-terminal NpuDnaE intein/C-terminal PAH fragment (PAH C-term(G)); or both (PAH N-term(G) and PAH C-term(G)). Cells transfected with (GTP-CH1)-IRES-GOI with full-length PAH (FL PAH(G)) or control (mock) are also shown. FIG. 11A shows the viability of cells following selection in tyrosine-deficient media containing no cofactors. FIG. 11B shows the viable cell density (VCD) of cells following selection in tyrosine-deficient media containing no cofactors.

FIGS. 12A-12B show the growth of cells transfected with both the N-terminal PAH fragment/N-terminal NpuDnaE intein and the C-terminal NpuDnaE intein/C-terminal PAH fragment, where (GTP-CH1)-IRES-GOI is co-expressed on just the N-terminal plasmid (PAH N-term(G)+PAH C-term), the C-terminal plasmid (PAH N-term+C-term(G)), or both (PAH N-term(G)+PAH C-term(G)). FIG. 12A shows the viability of cells following selection in tyrosine-deficient media containing no cofactors. FIG. 12B shows the viable cell density (VCD) of cells following selection in tyrosine-deficient media containing no cofactors.

FIGS. 13A-13B show vector diagrams for a representative split-intein design for glutamine synthetase (GS). FIG. 13A shows the N-terminal GS fragment ending at the Cys53 split point, fused to the N-terminal NpuDnaE intein fragment. FIG. 13B shows the C-terminal NpuDnaE intein fragment fused to the C-terminal GS fragment at the Cys53 split point.

FIG. 14 shows the vector diagrams for a exemplary split-intein design for thymidylate synthase (TYMS). FIG. 14A shows the N-terminal TYMS fragment ending at the Cys161 split point, fused to the N-terminal NpuDnaE intein fragment. FIG. 14B shows the C-terminal NpuDnaE intein fragment fused to the C-terminal TYMS fragment at the Cys161 split point.

FIGS. 15A-15B show vector diagrams for exemplary split-intein designs for constructs encoding each of the split intein/PAH fragments. FIG. 15A shows vector diagrams for a construct 1 (C1) encoding for AAV Rep (Rep2BFP CODE) and Cap (Cap5) proteins and a construct 2 (C2) encoding for a gene of interest (e.g., GFP AAV), where both constructs further comprise a PAH fragment operably linked to a portion of an intein (e.g., a C-terminal portion of an intein+C-terminal PAH fragment in C1 and an N-terminal portion of an intein+N-terminal PAH fragment in C2). FIG. 15B shows vector diagrams for a construct 3 (C3) encoding for AAV Rep (Rep2BFP CODE) and Cap (Cap5) proteins and a construct 4 (C4) encoding for a gene of interest (e.g., GFP AAV), where both constructs further comprise sequences encoding for P2A (a self-cleaving peptide) and GTP-CH1 (to facilitate tyrosine production and support cell growth in the absence of exogenously added cofactors). Both constructs additionally further comprise PAH fragments operably linked to portions of split inteins (e.g., a C-terminal portion of an intein+C-terminal PAH fragment in C3 and an N-terminal portion of an intein+N-terminal PAH fragment in C4).

FIG. 16 show vector diagrams for exemplary split-intein designs for constructs encoding each of the split intein/PAH fragments. Construct 5 (C5) shows a vector diagram for a construct encoding for AAV Rep (Rep2BFP CODE) and Cap (Cap5) proteins in a tail-to-tail orientation with a C-terminal portion of an intein+C-terminal PAH fragment, a P2A (a self-cleaving peptide), and GTP-CH1 (to facilitate tyrosine production and support cell growth in the absence of exogenously added cofactors) under the control of a EFla WT promoter. Construct 6 (C6) shows a vector diagram for a construct encoding for AAV Rep (Rep2BFP CODE) and Cap (Cap5) proteins in a tail-to-tail orientation with a C-terminal portion of an intein+C-terminal PAH fragment, a P2A, and GTP-CH1 under the control of a EFla mutant promoter (TATGTA). Construct 7 (C7) shows a vector diagram for a construct encoding for AAV Rep (Rep2BFP CODE) and Cap (Cap5) proteins in a tail-to-tail orientation with a C-terminal portion of an intein+C-terminal PAH fragment under the control of a EFla WT promoter. Construct 8 (C8) shows a vector diagram for a construct encoding for AAV Rep (Rep2BFP CODE) and Cap (Cap5) proteins in a tail-to-tail orientation with a C-terminal portion of an intein+C-terminal PAH fragment under the control of a EF1a mutant promoter (TATGTA). Construct 9 (C9) shows a vector diagram for a construct encoding for AAV Rep (Rep2BFP CODE) and Cap (Cap5) proteins in a tail-to-tail orientation with a C-terminal portion of an intein+C-terminal PAH fragment under the control of a EFla WT promoter. C9 further encodes for GTP-CH1 under the control of a CMV promoter in head-to-head orientation with the C-terminal portion of an intein+C-terminal PAH fragment under the control of the EFla WT promoter. Construct 10 (C10) shows a vector diagram for a construct encoding for AAV Rep (Rep2BFP CODE) and Cap (Cap5) proteins in a tail-to-tail orientation with a C-terminal portion of an intein+C-terminal PAH fragment under the control of a EFla mutant promoter (TATGTA). C10 further encodes for GTP-CH1 under the control of a CMV promoter in head-to-head orientation with the C-terminal portion of an intein+C-terminal PAH fragment under the control of the EF1a mutant promoter (TATGTA). Construct 11 (C11) shows a vector diagram for a construct encoding for a gene or payload of interest (e.g., GFP AAV) in a head-to-head orientation with a N-terminal portion of an intein+N-terminal PAH fragment under the control of EF1a mutant promoter (TATGTA). Construct 12 (C12) shows a vector diagram for a construct encoding for a gene or payload of interest (e.g., GFP AAV) in a head-to-head orientation with a N-terminal portion of an intein+N-terminal PAH fragment, a P2A, and GTP-CH1 under the control of EF1a mutant promoter (TATGTA).

FIG. 17 shows the viable cell density (VCD) of cells transfected with different constructs from FIGS. 15A, 15B, and 16 following 7 days, 10 days, or 14 days selection in tyrosine-deficient media containing 200 uM co-factor (BH2) (selection media).

FIG. 18 shows the viable cell density (VCD) of cells transfected with different constructs from FIGS. 15A, 15B, and 16 following 7 days, 10 days, or 14 days selection in tyrosine-deficient media containing no cofactors (selection media).

FIG. 19 shows the viable cell density (VCD) of cells transfected with different constructs from FIGS. 15A, 15B, and 16 following 7 days, 10 days, or 14 days in tyrosine-deficient media containing no cofactors (selection media).

FIG. 20 shows the viable cell density (VCD) of cells transfected with different constructs from FIGS. 15A, 15B, and 16 following 7 days, 10 days, or 14 days in tyrosine-deficient media containing no cofactors (selection media). The boxed bars on the graph indicate the cells having the highest percentage of cells expressing EGFP (Top EGFP+) among the different construct combinations tested.

FIG. 21 shows an exemplary flow cytometry plot for EGFP expression (x-axis) of cells from the boxed bars on the graph of FIG. 20.

FIG. 22 shows flow cytometry plots for EGFP expression (x-axis; percentage of EGFP+ cells shown in lower right corner) for cells transfected with C4 and C3 (top plots) or C12 and C6 (bottom plots). Cells were then grown in selective media not having tyrosine (left column), for 3 days in complete media having tyrosine (middle column), or for 11 days in complete media having tyrosine (right column).

FIG. 23 shows a generic schematic of splitting a glutamine synthetase (GS) protein into two different constructs, in which the split occurs at Cys residue within the GS protein. For example, a split can occur at Cys53, Cys183, Cys229, and Cys252 for producing a split GS protein. More specifically, one schematic shows a split-GS N-Term Module comprising a sequence encoding the N terminus of a split GS (which can be split at a position immediately N-terminal to the Cys residue (Met1 to CysN-1)) and an N terminus of a split intein (Dna-NpuE N-terminus) as well as a sequence encoding GFP AAV. The second schematic shows a split-GS C-Term Module comprising a sequence encoding a C terminus of the split intein (Dna-NpuE C-terminus) and the C terminus of the split GS (which starts at the Cys N residue of the split-GS N-Term Module (CysN to End)) and as well as a sequence encoding the Rep and Cap proteins (Rep2 and Cap5) for AAV production. Exemplary Cys residue N can be Cys53, Cys183, Cys229, or Cys252.

FIG. 24 shows the viable cell density (VCD) of cells transfected with a plasmid coding for a split-GS N-Term Module and a plasmid coding for a split-GS C-Term Module, only a plasmid coding for the split-GS N-Term Module, only a plasmid coding for the split-GS C-Term Module, no plasmids encoding split-GS modules, or a plasmid coding for a split-Blasticidin N-Term Module and a plasmid coding for a split-Blasticidin C-Term Module (a split of a protein encoding for a Blasticidin resistance is used in place of the split GS). These cells were produced by transfecting a GS KO parent cell (parental viral producer cell (VPC)) with a construct coding for helper proteins and a puromycin resistant protein (helper construct). These cells were cultured in media comprising puromycin to select for integration of the helper construct. Next, these cells were transfected with a plasmid coding for a split-GS N-Term Module and a plasmid coding for a split-GS C-Term Module, only a plasmid coding for the split-GS N-Term Module, only a plasmid coding for the split-GS C-Term Module, no plasmids encoding split-GS modules, or a plasmid coding for a split-Blasticidin N-Term Module and a plasmid coding for a split-Blasticidin C-Term Module. These cells were cultured in media having no glutamine (selection media) and then VCD was measured at various time points out to 15 days after switching to the selection media. Different split GS modules were tested as indicated: top left graph tested a split at Cys53; top right tested a split at Cys183; bottom left tested a split at Cys229; and bottom right tested a split at Cys252.

FIG. 25 shows the percentage of cells expressing EGFP in the cells transfected with a plasmid coding for a split-GS N-Term Module and a plasmid coding for a split-GS C-Term Module compared to a plasmid coding for a split-Blasticidin N-Term Module and a plasmid coding for a split-Blasticidin C-Term Module (positive control) or a parental VPC not transfected with any plasmids (negative control). The split GS modules tested were, from left to right, a split at Cys53, a split at Cys183, a split at Cys229, or a split at Cys252.

FIG. 26 shows titer of virions (vg/ml) as measured by qPCR after induction of the cells having integrated helper constructs and the termini of the split GS module (P1-Puro/P2-SplitGS) in which split GS modules tested were, from left to right, a split at Cys53, a split at Cys183, a split at Cys229, or a split at Cys252, as described in FIG. 24; cells transfected with a helper construct coding for a GS protein instead of puromycin resistance gene followed by transfection with constructs coding for a split blasticidin resistance protein instead of a split GS protein (P1-GS/P2-SplitBlast); cells transfected with a helper construct coding for a puromycin resistance gene followed by transfection with constructs coding for a split blasticidin resistance protein instead of a split GS protein (T42); or a negative control. Titer was measured at either day 3 post-induction of virion or day 5 post-induction of virion.

### DETAILED DESCRIPTION

The present disclosure provides compositions for leveraging metabolic markers for selection of cells within a population of cells. Cells selected by utilizing the compositions and methods disclosed herein retain exogenous nucleic acid of interest. Exogenous nucleic acids of interest encompassed herein include polynucleotide constructs encoding for various components needed for adeno-associated virus (AAV) production. In some embodiments, the compositions disclosed herein encompass exogenous nucleic acid constructs encoding for (a) adenoviral helper proteins such as E1, E2A, E4A, VA-RNA, or any combination thereof and (b) a functional enzyme capable of metabolizing and producing a molecule necessary for cell growth. In some embodiments, the compositions disclosed herein encompass exogenous nucleic acid constructs encoding for (a) adenoviral Rep proteins, adenoviral Cap proteins, or any combination thereof and (b) a functional enzyme capable of metabolizing and producing a molecule necessary for cell growth. In some embodiments, the compositions disclosed herein encompass exogenous nucleic acid constructs encoding for (a) a payload such as any therapeutic payload disclosed herein and (b) a functional enzyme capable of producing a molecule necessary for cell growth in selection conditions. In some embodiments, the compositions disclosed herein encompass a set of exogenous nucleic acid constructs, including (i) a first exogenous nucleic acid construct encoding for (a) adenoviral Rep proteins, adenoviral Cap proteins, or any combination thereof and (b) a portion of a split intein linked to a portion of a functional enzyme capable of producing a molecule necessary for cell growth and (ii) a second exogenous nucleic acid construct encoding for (a) adenoviral helper proteins such as E1, E2A, E4A, VA-RNA, or any combination thereof and (b) a second portion of the split intein linked to a second portion of the functional enzyme capable of producing a molecule necessary for cell growth.

In some embodiments, the compositions disclosed herein encompass a set of exogenous nucleic acid constructs, including (i) a first exogenous nucleic acid construct encoding for (a) adenoviral Rep proteins, adenoviral Cap proteins, or any combination thereof and (b) a portion of a split intein linked to a portion of a functional enzyme capable of producing a molecule necessary for cell growth and (ii) a second exogenous nucleic acid construct encoding for (a) a payload such as any therapeutic payload disclosed herein and (b) a second portion of the split intein linked to a second portion of the functional enzyme capable of producing a molecule necessary for cell growth.

In some embodiments, the compositions disclosed herein encompass a set of exogenous nucleic acid constructs, including (i) a first exogenous nucleic acid construct encoding for (a) a payload such as any therapeutic payload disclosed herein and (b) a portion of a split intein linked to a portion of a functional enzyme capable of producing a molecule necessary for cell growth and (ii) a second exogenous nucleic acid construct encoding for (a) adenoviral helper proteins such as E1, E2A, E4A, VA-RNA, or any combination thereof and (b) a second portion of the split intein linked to a second portion of the functional enzyme capable of producing a molecule necessary for cell growth.

Also provided herein are methods for transfecting cells with any combination of the exogenous nucleic acid constructs disclosed herein to leverage metabolic cell selection of cells expressing Rep and Cap proteins, adenoviral helper proteins and, optionally, a payload. In some embodiments, a single selective pressure is used to select for two exogenous nucleic acid constructs. For example, provided herein are cells transfected with a first exogenous nucleic acid construct encoding for adenoviral helper proteins and a first functional enzyme capable of enabling metabolic marker based selection as described through this disclosure. Cells successfully transfected with adenoviral helper proteins are selected for with a first single selective pressure. Subsequently, these cells are transfected with a set of exogenous nucleic acid constructs, wherein one construct encodes for Rep and Cap proteins along with a first portion of a second functional enzyme linked to one portion of a split intein and the other construct encodes for a payload along with the second portion of the second functional enzyme linked to the second portion of the split intein. Upon transfecting cells with this set of exogenous nucleic acids, the second functional enzyme is fully reconstituted and cells having the fully reconstituted second functional enzyme are selected for with a second single selective pressure.

Thus, the compositions and methods disclosed herein offer the ability to perform metabolic marker-based selection of cells in multi-step transfections. In some embodiments, any of the compositions and methods disclosed herein can be combined with conventional antibiotic-based selection of cells.

### Definitions

Unless defined otherwise, all technical and scientific terms used herein have the meaning commonly understood by one of ordinary skill in the art to which the invention pertains.

The term “auxotroph or “auxotrophic” as used herein refers to a cell or cell line that requires a particular nutrient in order to grow. Cells can be naturally auxotrophic for a particular nutrient or can be engineered to be auxotrophic, for example, by knocking out a gene encoding an enzyme necessary for generating a metabolite that is essential for cell growth.

The term “selectable marker” as used herein refers to a gene that when expressed in a cell, permits the cell to be selected for retention and expression of the gene. In some embodiments, a selectable marker encodes an enzyme that allows the cell to grow in a medium lacking an essential nutrient. In some embodiments, a selectable marker encodes an enzyme that allows the cell to grow in the presence of a toxic agent (e.g., antibiotic, toxin).

The term “mammalian cell” as used herein refers to cells from humans and non-humans, including but not limited to humans, non-human primates, canines, felines, murines, bovines, equines, and porcines.

The term “recombinant cell” as used herein refers to a cell into which exogenous nucleic acid has been introduced.

The term “cell line” as used herein refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term “includes the progeny of the original cell which has been transfected. Thus, a “host cell” may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. A “host cell” as used herein may refer to any mammalian cell which is capable of functioning as an adenovirus packaging cell, i.e., expresses any adenovirus proteins essential to the production of AAV, such as HEK 293 cells and their derivatives (HEK293T cells, HEK293F cells), HeLa, A549, Vero, CHO cells or CHO-derived cells, and other packaging cells.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

As used herein, the term “intermediate cell line” refers to a cell line that contains the AAV rep and cap components integrated into the host cell genome or a cell line that contains the adenoviral helper functions integrated into the host cell genome.

As used herein, the term “packaging cell line” refers to a cell line that contains the AAV rep and cap components and the adenoviral helper functions integrated into the host cell genome or otherwise stably retained in the cell line (e.g., as an episome). A payload construct must be added to the packaging cell line to generate rAAV virions.

As used herein, the term “production cell line” refers to a cell line that contains the AAV rep and cap components, the adenoviral helper functions, and a payload construct. The rep and cap components and the adenoviral helper functions are integrated into the host cell genome or otherwise stably retained in the cell line (e.g., as an episome). The payload construct can be stably integrated into the host cell genome or transiently transfected. rAAV virions can be generated from the production cell line upon the introduction of one or more triggering agents in the absence of any plasmid or transfection agent.

As used herein, the term “downstream purification” refers to the process of separating rAAV virions from cellular and other impurities. Downstream purification processes include chromatography-based purification processes, such as ion exchange (IEX) chromatography and affinity chromatography.

The term “prepurification yield” refers to the rAAV yield prior to the downstream purification processes. The term “postpurification yield” refers to the rAAV yield after the downstream purification processes. rAAV yield can be measured as viral genome (vg)/L.

The encapsidation ratio of a population of rAAV virions can be measured as the ratio of rAAV viral particle (VP) to viral genome (VG). The rAAV viral particle includes empty capsids, partially full capsids (e.g., comprising a partial viral genome), and full capsids (e.g., comprising a full viral genome).

The F:E ratio of a population of rAAV virions can be measured as the ratio of rAAV full capsids to empty capsids. The rAAV full capsid particle includes partially full capsids (e.g., comprising a partial viral genome) and full capsids (e.g., comprising a full viral genome). The empty capsids lack a viral genome.

The potency or infectivity of a population of rAAV virions can be measured as the percentage of target cells infected by the rAAV virions at a multiplicity of infection (MOI; viral genomes/target cell). Exemplary MOI values are 1×10¹, 1×10², 2×10³, 5×10⁴, or 1×10⁵ vg/target cell. An MOI can be a value chosen from the range of 1×10¹ to 1×10⁵ vg/target cell.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. The use of the term “vector” throughout this specification refers to either plasmid or viral vectors, which permit the desired components to be transferred to the host cell via transfection or infection. For example, an adeno-associated viral (AAV) vector is a plasmid comprising a recombinant AAV genome. In some embodiments, useful vectors are contemplated to be those vectors in which the nucleic acid segment to be transcribed is positioned under the transcriptional control of a promoter.

The phrases “operatively positioned,” “operatively linked,” “under control” or “under transcriptional control” means that the promoter is in the correct location and orientation in relation to the nucleic acid to control RNA polymerase initiation and expression of the gene.

The term “expression vector or construct” or “synthetic construct” means any type of genetic construct containing a nucleic acid in which part or all of the nucleic acid coding sequence is capable of being transcribed. In some embodiments, expression includes transcription and translation of the nucleic acid, for example, to generate a biologically-active polypeptide product from a gene or includes transcription of a functional RNA (e.g., guide RNA) from a transcribed nucleic acid sequence.

The term “payload”, “payload polynucleotide”, “expressible therapeutic polynucleotide,” or “expressible polynucleotide encoding a payload” refers to a polynucleotide that is encoded in an AAV genome vector (“AAV genome vector”) flanked by AAV inverted terminal repeats (ITRs). In some embodiments, the payload is a therapeutic payload (also referred to as a “therapeutic polynucleotide”). Such a polynucleotide payload is a payload that may include any one or combination of the following: a gene (e.g., a transgene), a tRNA suppressor, a guide RNA, or any other target binding/modifying oligonucleotide or derivative thereof, or payloads can include immunogens for vaccines, and elements for any gene editing machinery (DNA or RNA editing). Payloads can also include those that deliver a transgene encoding antibody chains or fragments that are amenable to viral vector-mediated expression. Payloads can also include those that deliver a gene encoding a protein that is amenable to viral vector-mediated expression. Payloads can also encode for detectable markers including, but not limited to, GFP, EGFP, BFP, RFP, or YFP.

An “rAAV vector” as used herein refers to an AAV vector comprising a polynucleotide sequence not of AAV origin (e.g., a polynucleotide heterologous to AAV), typically a sequence of interest for the genetic transformation of a cell. In some embodiments, the heterologous polynucleotide may be flanked by at least one, and sometimes by two, AAV inverted terminal repeat sequences (ITRs). The term rAAV vector encompasses both rAAV vector particles and rAAV vector plasmids. A rAAV vector may either be single-stranded (ssAAV) or self-complementary (scAAV). An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as a “rAAV vector particle” or simply an “rAAV vector”. Thus, production of rAAV particle necessarily includes production of rAAV vector, as such a vector is contained within an rAAV particle.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information.

Abbreviations used in this application include the following: 7,8-BH2 (7,8-dihydrobiopterin); BH4 ((6R)-5,6,7,8-tetrahydrobiopterin); DHFR (dihydrofolate reductase); GS (glutamine synthetase); IRES (internal ribosome entry site); PAH (phenylalanine hydroxylase); and TYMS (thymidylate synthase).

It must be noted that, as used in the specification and the appended claims, the singular forms “a,” “an” and “the” include plural referents unless the context clearly dictates otherwise.

Metabolic Selection

The disclosure provided herein relates to generating cells that are selected for retention of exogenous nucleic acid without the use of antibiotics or cellular toxins for selection. In a first aspect, provided herein are compositions and methods for metabolic selection.

In certain embodiments, exogenous nucleic acid that is introduced into host cells encodes an enzyme that is involved in the production of a molecule that is necessary for cell growth. Cells that retain the exogenous nucleic acid are selected for based on the ability of the cells to grow in medium that lacks the molecule necessary for cell growth. Metabolic selection is further described in US 2019/0078099 and US 2020/0056190, both of which are herein incorporated by reference in their entirety.

In some embodiments, the molecule necessary for cell growth is glutamine. The enzyme glutamine synthetase (GS) catalyzes the production of glutamine from glutamate. In some embodiments, exogenous nucleic acid encoding GS is introduced into host cells that do not endogenously express functional glutamine synthetase (GS). In some embodiments, provided herein are host cells that have been engineered to knockout GS. For example, gene editing tools (e.g., CRISPR/Cas systems including CRISPR/Cas9; TALENs; ZFNs; etc.) are used to generate engineered cell lines that are knocked out for GS and capable of viral production. In some embodiments, the host cells are HEK cells and their derivatives (e.g., HEK293 cells), AV459 cells, Vero cells, HeLa cells, and CHO cells, or any cells derived therefrom. In particular embodiments, cells that do not endogenously express functional GS are selected for retention of exogenous nucleic acid encoding GS based on the ability of the cells to grow in selection medium lacking glutamine Thus, the present disclosure provides host cells capable of viral production knocked out for GS and exogenous nucleic acid constructs encoding for GS. In some embodiments, an exogenous nucleic acid construct encodes for full length GS. In some embodiments, provided herein are a set of exogenous nucleic acid constructs, each encoding a portion of the GS enzyme. Said exogenous nucleic acid constructs also encode a portion of a split intein (e.g., the N-terminal fragment of the Nostoc punctiforme (Npu) split DnaE intein (NpuDnaE split intein) or the C-terminal fragment of the NpuDnaE split intein), wherein the portion of the split intein is linked to the portion of the GS enzyme.

In some embodiments, the molecule necessary for cell growth is thymidine. The endogenous enzyme thymidylate synthetase (TYMS) converts deoxyuridine monophosphate (dUMP) to deoxythymidine monosphosphate (dTMP). TYMS-deficient HEK293 cells cannot grow in the absence of thymidine. In some embodiments, exogenous nucleic acid encoding TYMS is introduced into host cells that do not endogeneously express functional TYMS. In some embodiments, provided herein are host cells that have been engineered to knockout TYMS. For example, gene editing tools (e.g., CRISPR/Cas systems including CRISPR/Cas9; TALENs; ZFNs; etc.) are used to generate engineered cell lines that are knocked out for TYMS and capable of viral production. In some embodiments, the host cells are HEK cells and their derivatives (e.g., HEK293 cells), AV459 cells, Vero cells, HeLa cells, and CHO cells, or any cells derived therefrom. In particular embodiments, cells that do not endogenously express functional TYMS are selected for retention of exogenous nucleic acid encoding TYMS based on the ability of the cells to grow in selection medium lacking thymidine. Thus, the present disclosure provides host cells capable of viral production knocked out for TYMS and exogenous nucleic acid constructs encoding for TYMS. In some embodiments, an exogenous nucleic acid construct encodes for full length TYMS. In some embodiments, provided herein are a set of exogenous nucleic acid constructs, each encoding a portion of the TYMS enzyme. Said exogenous nucleic acid constructs also encode for a portion of a split intein (e.g., the N-terminal fragment of the Nostoc punctiforme (Npu) split DnaE intein (NpuDnaE split intein) or the C-terminal fragment of NpuDnaE split intein), wherein the portion of the split intein is linked to the portion of the TYMS enzyme.

In some embodiments, the molecule necessary for cell growth is hypoxanthine or thymidine. The enzyme dihydrofolate reductase (DHFR) catalyzes a reaction necessary for the production hypoxanthine and thymidine. In some embodiments, exogenous nucleic acid encoding DHFR is introduced into host cells that do not endogenously express functional DHFR. In some embodiments, provided herein are host cells that have been engineered to knockout DHFR. For example, gene editing tools (e.g., CRISPR/Cas systems including CRISPR/Cas9; TALENs; ZFNs; etc.) are used to generate engineered cell lines that are knocked out for DHFR and capable of viral production. In some embodiments, the host cells are HEK cells and their derivatives (e.g., HEK293 cells), AV459 cells, Vero cells, HeLa cells, and CHO cells, or any cells derived therefrom. In particular embodiments, cells that do not endogenously express functional DHFR are selected for retention of exogenous nucleic acid encoding DHFR based on the ability of the cells to grow in selection media lacking hypoxanthine and thymidine. Thus, the present disclosure provides host cells capable of viral production knocked out for DHFR and exogenous nucleic acid constructs encoding for DHFR. In some embodiments, an exogenous nucleic acid construct encodes for full length DHFR. In some embodiments, provided herein are a set of exogenous nucleic acid constructs, each encoding a portion of the DHFR enzyme. Said exogenous nucleic acid constructs also encode for a portion of a split intein (e.g., the N-terminal fragment of the Nostoc punctiforme (Npu) split DnaE intein (NpuDnaE split intein) or the C-terminal fragment of NpuDnaE split intein), wherein the portion of the split intein is linked to the portion of the DHFR enzyme.

In some embodiments, the molecule necessary for cell growth is tyrosine. The enzyme phenylalanine hydroxylase (PAH) catalyzes the conversion of phenylalanine to tyrosine. In some embodiments, exogenous nucleic acid encoding PAH is introduced into host cells that do not endogenously express functional PAH. In some embodiments, these host cells are capable of viral production, are naturally auxotrophic for one or more nutrients (e.g., tyrosine), and lack endogenous functional PAH. In particular embodiments, cells that do not endogenously express functional PAH are selected for retention of exogenous nucleic acid encoding PAH based on the ability of the cells to grow in selection media lacking tyrosine. In some embodiments, metabolic selection media comprises a cofactor or cofactor precursor. In particular embodiments, the cofactor or cofactor precursor is tetrahydrobiopterin (BH4) or 7,8-dihydrobiopterin (7,8-BH2). Thus, the present disclosure provides naturally auxotrophic host cells capable of viral production and lacking endogenous PAH, exogenous nucleic acid constructs encoding for PAH, and a cofactor (e.g., BH4 or BH2). In some embodiments, an exogenous nucleic acid construct encodes for full length PAH. In some embodiments, provided herein are a set of exogenous nucleic acid constructs, each encoding a portion of the PAH enzyme. Said exogenous nucleic acid constructs also encode for a portion of a split intein (e.g., the N-terminal fragment of the Nostoc punctiforme (Npu) split DnaE intein (NpuDnaE split intein) or the C-terminal fragment of NpuDnaE split intein), wherein the portion of the split intein is linked to the portion of the PAH enzyme.

Co-factors (e.g., BH4) that may be needed for certain selectable marker systems disclosed herein (e.g, PAH) can be supplemented in multiple ways. In some embodiments, the present disclosure provides for exogenous supplementation of a cofactor (e.g., BH4) by addition of the cofactor to the culture media. In some embodiments, the present disclosure provides for exogenous supplementation of a cofactor (e.g., BH4) by encoding for the cofactor on one of polynucleotide constructs encoding for PAH. In some embodiments, the present disclosure circumvents exogenous addition of the cofactor by instead encoding for an enzyme that converts a first molecule into the cofactor. For example, the polynucleotide constructs disclosed herein may encode for a full length or split PAH system and also further encode for GTP cyclohydrolase I (GTP-CH1), an enzyme in the GTP to BH4 conversion pathway. The resulting overexpression of GTP-CH1 in tandem with PAH can result in sufficient production of tyrosine to facilitate cell growth and maintenance of cell viability without the addition of exogenous BH4.

The compositions and methods as described herein for therapeutics using metabolic selection can provide increased safety, processing efficacy, and tunability compared to therapeutics using antibiotic selection. For example, using metabolic selection increases therapeutic safety by decreasing or eliminating the risk of packaging an antibiotic resistance gene in the therapeutic. As another example, using metabolic selection put less pressure on cells during selection relying on the production of nutrient (e.g., a metabolite) compared to the pressure of overcoming toxicity for selection using an antibiotic. As another example, metabolic selection allows for greater tunability of the copy number of constructs integrated into a cell during selection (e.g., by titrating inhibitors, tuning the strength of the promoter operably linked to the selectable marker, or mutating the selectable marker to tune activity of the selectable marker) compared to antibiotic selection that relies on titrating antibiotics.

Split Inteins for Metabolic Selection

Inteins

The disclosure provided herein relates to use of split intervening proteins (inteins) for metabolic selection. Inteins auto catalyze a protein splicing reaction that results in excision of the intein and joining of the flanking amino acids (extein sequences) via a peptide bond. Inteins exist in nature as a single domain within a host protein or, less frequently, in a split form. For split inteins, the two separate polypeptide fragments of the intein must associate in order for protein trans-splicing to occur to excise the intein. Split intein systems are described in: Cheriyan et al, J. Biol. Chem 288: 6202-6211 (2013); Stevens et al, PNAS 114: 8538-8543 (2017); Jillette et al., Nat Comm 10: 4968 (2019); US 2020/0087388 A1; and US 2020/0263197 A1.

In the disclosure provided herein, split inteins are used to catalyze the joining of two fragments (e.g., an N-terminal fragment and a C-terminal fragment) of a selectable protein, such as any one of the enzymes disclosed herein (e.g., PAH, GS, TYMS, DHFR). Split inteins may be naturally occurring or engineered.

Naturally occurring split inteins are found within the DnaE and DnaB genes of cyanobacteria. DnaE inteins of the present disclosure include, but are not limited to, the Nostoc punctiforme (Npu) DnaE intein and the Synechocystis species, strain PCC6803 (Ssp) DnaE intein. In some embodiments, an exogenous nucleic acid construct disclosed herein encodes for the N-terminal fragment of Npu DnaE intein linked to a first portion of any enzyme disclosed herein (e.g., PAH, GS, TYMS, DHFR). In further embodiments, a second exogenous nucleic acid construct disclosed herein encodes for the C-terminal fragment of Npu DnaE intein linked to a second portion of the enzyme. In some embodiments, the N-terminal fragment of Npu DnaE item comprises at least 80%, 90%, 95%, 98%, 99%, or 100% sequence identity to SEQ ID NO: 53. In some embodiments, the C-terminal fragment of Npu DnaE item comprises at least 80%, 90%, 95%, 98%, 99%, or 100% sequence identity to SEQ ID NO: 54. In some embodiments, an exogenous nucleic acid construct disclosed herein encodes for the N-terminal fragment of Ssp DnaE intein linked to a first portion of any enzyme disclosed herein (e.g., PAH, GS, TYMS, DHFR). In further embodiments, a second exogenous nucleic acid construct disclosed herein encodes for the C-terminal fragment of Ssp DnaE intein linked to a second portion of the enzyme. These exogenous nucleic acid constructs may further encode for components needed for AAV production (e.g., Rep and Cap proteins, adenoviral helper proteins) or payloads (e.g., any therapeutic payload disclosed herein).

In some embodiments, split inteins are engineered. Engineered split inteins of the present disclosure include, but are not limited to, the consensus DnaE intein (Cfa) (see, e.g., Stevens, et al., J Am Chem Soc. 138: 2162-2165 (2016).). In some embodiments, engineered split inteins may be modified DnaB inteins.

In some embodiments, the N-terminal fragment of Npu DnaE intein linked to a second portion of the enzyme comprises at least 80%, 85%, 90%, 95%, 98%, 99%, or 100% sequence identity to SEQ ID NO: 2, 4, 6, 8, 24, 26, 28, 30, 32, 35, 37, 39, or 41. In some embodiments, the C-terminal fragment of Npu DnaE intein linked to a second portion of the enzyme comprises at least 80%, 85%, 90%, 95%, 98%, 99%, or 100% sequence identity to SEQ ID NO: 3, 5, 7, 9, 25, 27, 29, 31, 33, 36, 38, 40, or 42.

Polynucleotides of Interest

Provided herein are compositions and methods for generating recombinant cells selected for retention of at least two exogenous nucleic acid constructs. In some embodiments, at least two exogenous nucleic acid constructs comprise a first polynucleotide of interest and a second polynucleotide of interest. In some embodiments, said first and second polynucleotides of interest are any of the payloads disclosed herein.

In some embodiments of the present disclosure the polynucleotide of interest is a gene or transgene encoding a protein of interest. Examples of proteins of interest include, but are not limited to, therapeutic proteins (e.g., enzymes, hormones, transcription factors), AAV Rep and Cap proteins, and adenoviral helper proteins (e.g., E1, E2A, E4A, VA-RNA, or any combination thereof). In some embodiments, a polynucleotide of interest comprises at least 80%, 85%, 90%, 95%, 98%, 99%, or 100% sequence identity to any of SEQ ID NO: 46-SEQ ID NO: 49.

In some embodiments, the polynucleotide of interest is a non-coding RNA and may be a therapeutic payload. Examples of non-coding RNA include, but are not limited to, guide RNA (gRNA), antisense RNA (asRNA), microRNA (miRNA), short-interfering RNA (siRNA), short-hairpin RNA (shRNA), and transfer RNA (tRNA). In some embodiments, the polynucleotide of interest encodes a therapeutic payload. For example, a therapeutic payload disclosed herein may include a guide RNA (gRNA) or a tRNA suppressor. In certain embodiments, the guide RNA directs RNA editing. In some embodiments, the guide RNA directs Cas-mediated DNA editing. In some embodiments, the transgene encodes for progranulin. In some embodiments, the tRNA suppressor is capable of suppressing an opal stop codon. In some embodiments, the tRNA suppressor is capable of suppressing an ochre stop codon. In some embodiments, the tRNA suppressor is capable of suppressing an amber stop codon. In some embodiments, the payload is a homology element for homolog-directed repair. In some embodiments, the payload refers to a polynucleotide pacakaged for gene therapy.

Payloads can also include those that deliver transgene-encoding antibody chains or fragments that are amenable to viral vector-mediated expression (also referred to as “vectored antibody” or “vectorized antibody” for gene delivery). See, e.g., Curr Opin HIV AIDS. 2015 May; 10(3): 190-197, describing vectored antibody gene delivery for the prevention or treatment of HIV infection and U.S. Pat. No. 10,780,182, describing AAV delivery of trastuzumab (Herceptin) for treatment of HER2+ brain metastases.

In some embodiments, the polynucleotide of interest encodes for multiple copies of the same payload. In some embodiments, the polynucleotide of interest encodes for different payloads. In some embodiments, the polynucleotide of interest encodes for any marker. Non-limiting examples of markers include fluorescent proteins, such as GFP, EGFP, RFP, BFP, YFP, or any combination thereof.

In another embodiment, provided herein are compositions and methods for the production of recombinant antibodies. In some embodiments, the first polynucleotide of interest encodes an antibody heavy chain. In some embodiments, the second polynucleotide of interest encodes an antibody light chain. In some embodiments, the polynucleotide of interest encodes a variable region of an antibody heavy chain or light chain. In some embodiments, the polynucleotide of interest encodes a constant region of an antibody.

In yet another embodiment, provided herein are compositions and methods for generating recombinant cells that express reporter proteins. In some embodiments, the recombinant cells are selected for retention of nucleic acid constructs encoding at least two reporter proteins using a single selective pressure. In some embodiments, the reporter protein is a membrane transporter. In some embodiments, the reporter protein is a drug-metabolizing enzyme.

Selectable Marker

The recombinant cells of the present disclosure are selected based on their expression of a functional selectable protein encoded by a selectable marker. A selectable marker confers a trait suitable for artificial selection.

In some embodiments, the selectable marker encodes a selectable protein necessary for synthesis of an essential nutrient. Examples of such metabolic selectable markers include, but are not limited to, genes encoding dihydrofolate reductase (DHFR), glutamine synthetase (GS), thymidylate synthetase (TYMS), and phenylalanine hydroxylase (PAH). In some embodiments, PAH comprises at least 80%, 85%, 90%, 95%, 98%, 99%, or 100% sequence identity to SEQ ID NO: 1. In some embodiments, GS comprises at least 80%, 85%, 90%, 95%, 98%, 99%, or 100% sequence identity to SEQ ID NO: 23. In some embodiments, TYMS comprises at least 80%, 85%, 90%, 95%, 98%, 99%, or 100% sequence identity to SEQ ID NO: 34.

In some embodiments, the selectable marker encodes a selectable protein that confers resistance to a particular antibiotic or class of antibiotics. Examples of such antibiotic resistance genes include, but are not limited to, genes encoding proteins that confer resistance to ampicillin, blasticidin, bleomycin, carbenicillin, erythromycin, hygromycin, kanamycin, and puromycin.

In some embodiments, a full-length selectable marker is expressed under control of a single promoter. In some embodiments, a selectable marker is produced by joining a first portion and a second portion of a selectable marker in a cell, wherein the first and second portions are separately transcribed gene fragments of the full-length selectable marker.

With reference to a full-length selectable protein encoded by a first portion and a second portion of a selectable marker, in some embodiments the first portion of the selectable marker encodes an N-terminal fragment of the selectable protein. In some embodiments, the second portion of the selectable marker encodes a C-terminal fragment of the selectable protein. Accordingly, in some embodiments, a first portion of a selectable marker encodes a N-terminal fragment of phenylalanine hydroxylase (PAH) and a second portion of a selectable marker encodes a C-terminal fragment of PAH.

In some embodiments, a full-length, functional selectable protein is produced by joining a first portion of a selectable protein and a second portion of a selectable protein. In some embodiments, the first portion of the selectable protein is a nonfunctional N-terminal fragment of the selectable protein. In some embodiments, the second portion of the selectable protein is a nonfunctional C-terminal fragment of the selectable protein. In particular embodiments, the nonfunctional N-terminal fragment is linked by a peptide bond to the nonfunctional C-terminal fragment to generate a functional selectable protein (e.g., PAH).

In some embodiments, a selectable marker is produced by joining a first, a second portion, and a third portion of a selectable marker in a cell, wherein the first, second, and third portions are separately transcribed gene fragments of the full-length selectable marker.

With reference to a full-length selectable protein encoded by a first, second, and third portion of a selectable marker, in some embodiments the first portion of the selectable marker encodes an N-terminal fragment of the selectable protein. In some embodiments, the second portion of the selectable marker encodes a central fragment of the selectable protein and the third portion of the selectable marker encodes a C-terminal fragment of the selectable protein. In some embodiments, the first, second, and third portions of the selectable protein are nonfunctional fragments of the selectable protein. In particular embodiments, the nonfunctional N-terminal and C-terminal fragments are separately linked by a peptide bond to the nonfunctional central fragment to generate a functional selectable protein (e.g., PAH).

In some embodiments, the first or second exogenous nucleic acid construct further encodes a helper enzyme, wherein expression of the helper enzyme facilitates growth of the host cell in conjunction with the functional enzyme upon application of the single selective pressure. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the first or second exogenous nucleic acid construct may increase helper enzyme levels thereby increasing production of the molecule required for cell growth, by, e.g., increasing levels of a co-factor required for enzyme activity. In some embodiments, the first or the second exogenous nucleic acid construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the GTP-CH1 produces the cofactor (6R)-5,6,7,8-tetrahydrobiopterin (BH4) that is required for conversion of phenylalanine to tyrosine. In some embodiments, the host cell is a cell that expresses or is genetically modified to express GTP-CH1. In some embodiments, expression of GTP-CH1 facilitates growth of the host cell in conjunction with functional PAH upon application of the single selective pressure. In some embodiments, the helper enzyme comprises at least 80%, 90%, 95%, 98%, 99%, or 100% sequence identity to SEQ ID NO: 10.

Promoters

In some embodiments, a promoter suitable for maintaining the desired transcriptional activity is selected for use in a nucleic acid construct. In some embodiments, selection of a particular promoter is used to tune expression. In some embodiments, a strong promoter is selected to drive high expression of an encoded protein or payload. For example, a strong promoter may be selected to drive high expression of a therapeutic protein or payload encoded by a polynucleotide of interest. In some embodiments, a weak promoter is selected to drive low expression of an encoded protein or payload. For example, a weak promoter may be selected to drive expression of PAH in order to increase the stringency of tyrosine selection.

Promoters of the present disclosure include, but are not limited to: CMV, EF-1 alpha, UBC, PGK, CAGG, SV40, TRE., U6, and U7. A CMV promoter can have at least 80%, 90%, 95%, 98%, 99%, or 100% sequence identity to SEQ ID NO: 45. An EF-1 alpha (also referred to as EF1a or WT EF1a) promoter can have at least 80%, 90%, 95%, 98%, 99%, or 100% sequence identity to SEQ ID NO: 44. In some embodiments, the promoter is a mutated promoter. A mutated promoter can increase expression of an encoded protein or payload compared to a promoter that is not mutated. A mutated promoter can decrease or attenuate expression of an encoded protein or a payload as compared to a promoter that is not mutated. A mutated promoter can be, for example, an attenuated EF-1 alpha promoter. The attenuated EF-1 alpha (also referred to as mutant or mutated EF1a) promoter can have at least 80%, 90%, 95%, 98%, 99%, or 100% sequence identity to SEQ ID NO: 43. In some embodiments, the attenuate EF-1 alpha promoter may drive expression of GS in order to increase the stringency of glutamine selection.

Cells

Cells of the present disclosure include host cells used for generating recombinant cells; stable recombinant cells for viral production; and cells selected for high expression of one or more polynucleotides of interest.

Any cell or cell line that is known in the art to produce rAAV particles can be used for the methods disclosed herein. In some embodiments, a method of producing rAAV particles or increasing the production of rAAV particles disclosed herein uses HeLa cells, HEK293 cells, HEK293 derived cells (e.g., primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. A subject host cell can also be made using a baculovirus to infect insect cells such as Sf9 cells, which produce AAV (see, e.g., U.S. Pat. Nos. 7,271,002 and 8,945,918). In some embodiments, a host cell is any cell capable of activating a p5 promoter of sequence encoding a Rep protein. In some embodiments, a method disclosed herein uses HEK293 cells. In some embodiments, a method disclosed herein uses HEK293 cells adapted for growth in suspension culture.

In some embodiments, a cell culture disclosed herein is a suspension culture. In some embodiments, a cell culture disclosed herein is a suspension culture comprising HEK293. In some embodiments, a cell culture disclosed herein is a suspension culture comprising HEK293 cells adapted for growth in suspension culture. In some embodiments, a cell culture disclosed herein comprises a serum-free medium, an animal-component free medium, or a chemically defined medium. In some embodiments, a cell culture disclosed herein comprises a serum-free medium. In some embodiments, suspension-adapted cells are cultured in a shaker flask, a spinner flask, a cellbag, or a bioreactor.

In some embodiments, a cell culture disclosed herein comprises cells attached to a substrate (e.g., microcarriers) that are themselves in suspension in a medium. In some embodiments, the cells are HEK293 cells.

In some embodiments, a cell culture disclosed herein is an adherent culture. In some embodiments, a cell culture disclosed herein is an adherent culture comprising HEK293. In some embodiments, a cell culture disclosed herein comprises a serum-free medium, an animal-component free medium, or a chemically defined medium. In some embodiments, a cell culture disclosed herein comprises a serum-free medium.

In some embodiments, a cell culture disclosed herein comprises a high-density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

Cell lines for use as packaging cells include insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al., 1989, J. Virol. 63:3822-3828; Kajigaya et al., 1991, Proc. Nat'l. Acad. Sci. USA 88: 4646-4650; Ruffing et al., 1992, J. Virol. 66:6922-6930; Kimbauer et al., 1996, Virol. 219:37-44; Zhao et al., 2000, Virol. 272:382-393; and Samulski et al., U.S. Pat. No. 6,204,059.

For example, virus capsids according to the invention can be produced using any method known in the art, e.g., by expression from a baculovirus (Brown et al., (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, the present invention provide for a method of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculavirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA

Host Cells

As described herein, host cells are cells into which exogenous nucleic acid is introduced, thereby generating recombinant cells. Host cells of the present invention are eukaryotic cells. In some embodiments, host cells are mammalian cells. Examples of host cells include, but are not limited to, HEK cells and their derivatives (e.g., HEK293 cells), AV459 cells, Vero cells, HeLa cells, and CHO cells or any cells derived therefrom.

In some embodiments, host cells are genetically altered cells or cell lines derived from HEK293, AV459, or Vero cells. In some embodiments, host cells are genetically altered HEK293 cells that have been engineered to knock out one or more functional genes. In particular embodiments, host cells are modified HEK293 cells or cell lines in which the dihydrofolate reductase (DHFR), glutamine synthetase (GS), and/or thymidylate synthase (TYMS) genes have been knocked out, generating DHFR and/or GS null HEK293 cells. Methods of generating DHFR and GS null HEK293 cells have been previously described (see, e.g., US 2019/0078099 A1).

In some embodiments, host cells are naturally auxotrophic for one or more nutrients. In particular embodiments, host cells are HEK293 cells that are naturally auxotrophic for tyrosine.

In typical embodiments, the host cells of the present disclosure can be selected for retention of exogenous nucleic acid by culturing the cells in a selection medium. In particular embodiments, HEK293 host cells are selected for retention of exogenous nucleic acid comprising PAH by culturing the cells in medium lacking tyrosine. Accordingly, the naturally tyrosine auxotrophic HEK293 cells only grow in medium lacking tyrosine if they express functional PAH and can thereby produce tyrosine.

Stable Cells

Cells and cell lines generated by the compositions and methods of the present disclosure are host cells into which one or more nucleic acid constructs has been stably integrated into the genome of the host cell, thereby generating stable cells or cell lines. In some embodiments the stable cells or cell lines are viral production cells.

In some embodiments, a polynucleotide construct is integrated into the genome using a transposon system comprising a transposase and transposon donor DNA. The transposase can be provided to a host cell with an expression vector or mRNA comprising a coding sequence encoding the transposase. The transposon donor DNA can be provided with a vector comprising transposon terminal inverted repeats (TIRs). The polynucleotide construct is cloned into the transposon donor vector between the TIRs. The host cell is cotransfected with an expression vector or mRNA encoding the transposase and the transposon donor vector containing the polynucleotide construct insert, wherein the polynucleotide construct is excised from the transposon donor vector and integrated into the genome of the host cell at a target transposon insertion site. Transposition efficiency may be improved in a host cell by codon optimization of the transposase, using engineered hyperactive transposases, and/or introduction of mutations in the transposon terminal repeats. Any suitable transposon system can be used including, without limitation, the piggyBac, To12, or Sleeping Beauty transposon systems. For a description of various transposon systems, see, e.g., Kawakami et al. (2007) Genome Biol. 8 Suppl 1 (Suppl 1):57, Tipanee et al. (2017) Biosci Rep. 37(6):BSR20160614, Yoshida et al. (2017) Sci Rep. 7:43613, Yusa et al. (2011) Proc. Natl. Acad. Sci. USA 108(4):1531-1536, Doherty et al. (2012) Hum. Gene Ther. 23(3):311-320; herein incorporated by reference in their entireties.

In some embodiments, a construct is integrated at a target chromosomal locus by homologous recombination using site-specific nucleases or site-specific recombinases. For example, a construct can be integrated into a double-strand DNA break at the target chromosomal site by homology-directed repair. A DNA break may be created by a site-specific nuclease, such as, but not limited to, a Cas nuclease (e.g., Cas9, Cpf1, or C2c1), an engineered RNA-guided FokI nuclease, a zinc finger nuclease (ZFN), a transcription activator-like effector-based nuclease (TALEN), a restriction endonuclease, a meganuclease, a homing endonuclease, and the like. Any site-specific nuclease that selectively cleaves a sequence at the target site for integration of the construct may be used. Targeted Genome Editing Using Site-Specific Nucleases: ZFNs, TALENs, and the CRISPR/Cas9 System (T. Yamamoto ed., Springer, 2015); Genome Editing: The Next Step in Gene Therapy (Advances in Experimental Medicine and Biology, T. Cathomen, M. Hirsch, and M. Porteus eds., Springer, 2016); Aachen Press Genome Editing (CreateSpace Independent Publishing Platform, 2015); herein incorporated by reference in their entireties.

The construct sequence to be integrated is flanked by a pair of homology arms responsible for targeting the construct to the target chromosomal locus. A 5′ homology arm that hybridizes to a 5′ genomic target sequence and a 3′ homology arm that hybridizes to a 3′ genomic target sequence can be introduced into a polynucleotide construct. The homology arms are referred to herein as 5′ and 3′ (i.e., upstream and downstream) homology arms, which relates to the relative position of the homology arms in the polynucleotide construct. The 5′ and 3′ homology arms hybridize to regions within the target locus where the construct is integrated, which are referred to herein as the “5′ target sequence” and “3′ target sequence,” respectively.

In certain embodiments, the corresponding homologous nucleotide sequences in the genomic target sequence (i.e., the “5′ target sequence” and “3′ target sequence”) flank a specific site for cleavage and/or a specific site for integrating the construct. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate.

A homology arm can be of any length, e.g., 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc.

An RNA-guided nuclease can be targeted to a particular genomic sequence (i.e., genomic target sequence for insertion of a polynucleotide construct) by altering its guide RNA sequence. A target-specific guide RNA comprises a nucleotide sequence that is complementary to a genomic target sequence, and thereby mediates binding of the nuclease-gRNA complex by hybridization at the target site. For example, the gRNA can be designed selectively bind to the chromosomal target site where integration of the construct is desired. In certain embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of polynucleotide constructs by the HDR mechanism can be used for selective integration at a target chromosomal site, including CRISPR system type I, type II, or type III Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to selectively integrate a construct at a chromosomal target site as described herein.

The genomic target site may comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

The RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector) that is introduced into the host cell. Codon usage may be optimized to improve production of an RNA-guided nuclease in a particular cell or organism. For example, a nucleic acid encoding an RNA-guided nuclease can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells, the protein can be transiently, conditionally, or constitutively expressed in the cell.

Alternatively, site-specific recombinases can be used to selectively integrate a polynucleotide construct at a target chromosomal site. A target chromosomal site for integration of one or more polynucleotide constructs disclosed herein may include one or more transcriptionally active chromosomal sites. Examples of transcriptionally active chromosomal sites include DNasel hypersensitive sites (DHSs). A polynucleotide construct can be site-specifically integrated into the genome of a host cell by introducing a first recombination site into the construct and expressing a site-specific recombinase in the host cell. The target chromosomal site of the host cell comprises a second recombination site, wherein recombination between the first and second recombination sites mediated by the site-specific recombinase results in integration of the vector at the target chromosomal locus. The target chromosomal site may comprise either a recombination site native to the genome of the host cell or an engineered recombination site recognized by the site-specific recombinase. Various recombinases may be used for site-specific integration of vector constructs, including, but not limited to phi C31 phage recombinase, TP901-1 phage recombinase, and R4 phage recombinase. In some cases, a recombinase engineered to improve the efficiency of genomic integration at the target chromosomal site may be used. For a description of various site-specific recombinase systems and their use in site-specific recombination and genomic integration of constructs, see, e.g., U.S. Pat. No. 6,632,672; Olivares et al. (2001) Gene 278:167-176; Stoll et al. (2002) J. Bacteriol. 184(13):3657-3663; Thyagarajan et al. (2001) Mol. Cell Biol. 21(12):3926-3934; Sclimenti et al. (2001) Nucleic Acids Res. 29(24):5044-5051; Stark et al. (2011) Biochem. Soc. Trans. 39(2):617-22; Olorunniji et al. (2016) Biochem. J. 473(6):673-684; Birling et al. (2009) Methods Mol. Biol. 561:245-63; Garcia-Otin et al. (2006) Front. Biosci. 11:1108-1136; Weasner et al. (2017) Methods Mol. Biol. 1642:195-209; herein incorporated by reference in their entireties).

In some embodiments, one or more of the polynucleotide constructs are not integrated into the genome of the production host cell, and instead are maintained in the cell extrachromosomally. Examples of extrachromosomal polynucleotide constructs include those that persist as stable/persistent plasmids or episomal plasmids. In some embodiments, a construct comprises Epstein-Barr virus (EBV) sequences, including the EBV origin of replication, oriP, and the EBV gene, EBNA1, to provide stable extrachromosomal maintenance and replication of the construct. For a description of methods of using EBV sequences to stably maintain vectors extrachromosomally, see, e.g., Stoll et al. (2010) Mol. Ther. 4(2):122-129 and Deutsch et al. (2010) J. Virol. 84(5):2533-2546; herein incorporated by reference in their entireties. In some embodiments, the polynucleotide constructs of the present disclosure may be introduced into a cell in manner similar to the currently used triple-transfection method for production of rAAV virions.

In various embodiments, the stable cells or cell lines are propagated in selection media that lack a nutrient for which the host cell is auxotrophic. In particular embodiments, the stable cells or cell lines are propagated in media that lacks tyrosine.

In some embodiments, the present disclosure provides for compositions and methods of use thereof for metabolic marker-based selection of stable cell lines genomically integrated with constructs essential to adeno-associated virus (AAV) production. In place of, or in addition to, employing the incorporation of antibiotic resistance genes as a means for selecting for cells, the present disclosure provides for a means of selecting for stable cell lines using the full length or split selectable markers disclosed herein. In some embodiments, a suspension adapted viral production cell (VPC) is transfected with a construct encoding for AAV Rep and Cap proteins, a construct encoding for helper proteins, a construct encoding for a gene of interest, or a construct encoding for more than one of the aforementioned components. In further embodiments, the suspension adapted viral production cell is also transfected with a construct encoding for AAV Rep and Cap proteins, a construct encoding for helper proteins, a construct encoding for a gene of interest, or a construct encoding for more than one of the aforementioned components. In some embodiments, any one of the full length or split selectable marker systems disclosed herein (e.g., PAH, GS, DHFR, TYMS) is integrated into any of the above constructs in order to select for suspension adapted VPCs having all of the components (Rep and Cap proteins, helper proteins, and GOD needed for production of AAV encapsidating a payload. The suspension adapted VPCs may be first engineered to be knocked out for an enzyme (e.g., GS or DHFR) depending on the particular selectable marker system disclosed herein chosen to be integrated into the stable cell lines for AAV production. The suspension adapted VPCs may be grown in culture media lacking certain essential nutrients (e.g., glutamine or thymidine) depending on the particular selectable marker system disclosed herein chosen to be integated into the stable cell lines for AAV production. Examples of stable cell lines for AAV production further adapted to use the metabolic selectable markers disclosed herein are described in detail in Example 7-Example 12.

High-Expressing Cells

In some embodiments, stable cells or cell lines of the present disclosure are incubated in the presence of an inhibitor that amplifies the copy number of the selectable marker and, consequently, any polynucleotide of interest that is co-integrated or co-transfected with the selectable marker on the same construct. In some embodiments, a polynucleotide of interest that is co-integrated with the DHFR selectable marker in a DHFR null cell line is amplified by exposure to an inhibitor including, but not limited to methotrexate, ochratoxin A, alpha-methyl-tyrosine, alpha-methyl-phenylalanine, beta-2-thienyl-DL-alanine, and fenclonine. In some embodiments, a polynucleotide of interest that is co-integrated with the GS selectable marker in a GS null cell line is amplified by exposure to the inhibitor methionine sulfoximine. In particular embodiments, amplification of the polynucleotide of interest results in increased expression of the protein or nucleic acid encoded by the polynucleotide of interest, thereby generating cells or cell lines that highly express the protein or nucleic acid of interest.

In some embodiments, the functional selectable protein of the present disclosure is a mutated functional selectable protein having decreased protein activity compared to the protein activity of the functional selectable protein lacking the mutation. In some embodiments, the decreased activity of the mutated functional selectable protein results in an amplified copy number of the mutated functional selectable protein in a cell when cultured in selection media and, consequently any polynucleotide of interest that is co-integrated with or transfected on the same construct as the mutated functional selectable protein, as compared to a copy number of functional selectable protein lacking the mutation and consequently any polynucleotide of interest that is co-integrated with or transfected on the same construct as that functional selectable protein. For example, the functional selectable protein is GS, and a mutated functional selectable protein is GS having a mutation at R324C, R324S, or R341C compared to SEQ ID NO: 23. In some embodiments, the mutated GS has in decreased glutamine synthesis activity compared to GS without the mutation, and therefore, when a polynucleotide of interest is co-integrated with or transfected on the same construct as the mutated GS in a GS null cell line, the polynucleotide of interest is amplified when cultured in glutamine deficient media compared to a polynucleotide of interest that is co-integrated with or transfected on the same construct as GS without a mutation and cultured in glutamine deficient media. In some embodiments, the expression of a functional selectable protein of the present disclosure is driven by a mutated promoter having decreased promoter activity compared to the promoter activity of a promoter lacking the mutation. In some embodiments, the decreased activity of the mutated promoter results in an amplified copy number of the functional selectable protein in a cell when cultured in selection media and, consequently any polynucleotide of interest that is co-integrated with or transfected on the same construct as the functional selectable protein, as compared to a copy number of functional selectable protein driven by a promoter lacking the mutation and consequently any polynucleotide of interest that is co-integrated with or transfected on the same construct as that functional selectable protein. For example, the mutated promoter is an attenuated promoter, such as an attenuated EF1-alpha promoter. In some embodiments, a polynucleotide of interest that is co-integrated with or transfected on the same construct as the functional selectable protein driven by an attenuated EF1-alpha promoter is amplified when cultured in selection media compared to a polynucleotide of interest that is co-integrated with or transfected on the same construct as the functional selectable protein driven by a wild-type EF1-alpha promoter (e.g., SEQ ID NO: 44). In some embodiments, the attenuated EF-1 alpha (also referred to as mutant or mutated EF1a) promoter has at least 80%, 90%, 95%, 98%, 99%, or 100% sequence identity to SEQ ID NO: 43. In some embodiments, the wild-type EF-1 alpha (also referred to as wild-type EF1a) promoter has at least 80%, 90%, 95%, 98%, 99%, or 100% sequence identity to SEQ ID NO: 44.

In some embodiments, the methods as disclosed above for promoting high-expressing cells can be applied to methods of tuning the selection to achieve a desired copy number of a construct (e.g., a construct comprising the selectable marker or a portion of the selectable marker as described herein and the polynucleotide of interest). In some embodiments, the tuning of the copy number of the selectable marker and, consequently, any polynucleotide of interest that is co-integrated or co-transfected with the selectable marker on the same construct can include using a promoter having a desired strength (e.g., strong, medium, weak) that drives expression of the selectable marker for selection of a cell with a desired copy number of the selectable marker/polynucleotide. For example, a weak promoter can be used to produce a cell comprising a high copy number of the selectable marker/polynucleotide of interest. A strong promoter can be used to produce a cell comprising a low copy number of the selectable marker of interest. A weak promoter can be a mutated EF1alpha promoter, such as an attenuated EF1alpha promoter comprising SEQ ID NO: 43. An strong promoter can be the EF1alpha promoter comprising SEQ ID NO: 44. In some embodiments, the tuning of the copy number of the selectable marker and, consequently, any polynucleotide of interest that is co-integrated or co-transfected with the selectable marker on the same construct, can include using a selectable marker having a desired enzymatic activity (e.g., strong, medium, weak) for selection of a cell with a desired copy number of the selectable marker/polynucleotide. For example, a selectable marker mutated to have weak enzymatic activity can be used to produce a cell comprising a high copy number of the selectable marker/polynucleotide of interest. For example, a selectable marker having strong enzymatic activity can be used to produce a cell comprising a low copy number of the selectable marker/polynucleotide of interest. For example, the weak selectable marker can be a mutated GS, having a mutation at R324C, R324S, or R341C mutation as compared to SEQ ID NO: 23 (a selectable marker that is not mutated to have decreased enzymatic activity for this mutated GS is a GS having SEQ ID NO: 23). In some embodiments, the tuning of the copy number of the selectable marker and, consequently, any polynucleotide of interest that is co-integrated or co-transfected with the selectable marker on the same construct, can include culturing the cell with a specified concentration of inhibitor of the selectable marker for selection of a cell with a desired copy number of the selectable marker/polynucleotide. For example, the selectable marker can be GS and the cell can be cultured with a high concentration of methionine sulfoximine to produce a cell comprising a high copy number of the selectable marker/polynucleotide of interest. For example, the selectable marker can be GS and the cell can be cultured with a low concentration of methionine sulfoximine to produce a cell comprising a low copy number of the selectable marker/polynucleotide of interest. In some embodiments, the selectable maker is DHFR and the cell can be cultured with differing concentrations of methotrexate, ochratoxin A, alpha-methyl-tyrosine, alpha-methyl-phenylalanine, beta-2-thienyl-DL-alanine, or fenclonine to achieve the desired copy number of the selectable marker/polynucleotide of interest. In some embodiments, the selectable marker is a portion of selectable marker or a portion of a selectable protein as described herein. In some embodiments, a method of tuning for the copy number of a construct comprising a selectable marker or a portion of the selectable marker as described herein and the polynucleotide of interest in cell, comprises altering a promoter operably linked to the selectable marker or the portion of the selectable marker, altering the enzymatic activity of the selectable marker, or altering a concentration of an inhibitor of the selectable marker when culturing the cell for selection. The altering of the promoter can be to increase or decrease the strength of the promoter by mutating the promoter, or using a different promoter that has a different promoter strength. The altering enzymatic activity of the selectable marker can be to increase or decrease the enzymatic activity of the selectable marker, e.g., by mutating the selectable marker. The altering a concentration of an inhibitor of the selectable marker when culturing the cell for selection can be to increase or decrease the concentration of the inhibitor.

A selectable marker or a portion of a selectable marker can comprises at least 80%, 85%, 90%, 95%, 98%, 99%, or 100% sequence identity to SEQ ID NO: 1-SEQ ID NO: 9, or SEQ ID NO: 23-SEQ ID NO: 42. In some embodiments, the construct further comprises a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 10. In some embodiments, the selectable marker and helper enzyme of the construct comprises at least 80%, 85%, 90%, 95%, 98%, 99%, or 100% sequence identity to SEQ ID NO: 12-SEQ ID NO: 20. In some embodiments, the selection occurs in media comprising, for example, an antibiotic, or lacking nutrient required for cell growth accordingly for the selectable marker being used. In some embodiments, the media is supplemented with a cofactor or a cofactor precursor accordingly for the selectable marker being used and/or the helper enzyme being used.

Methods of Selecting Cells for Incorporation of Exogenous Nucleic Acid

In another aspect, methods of selecting cells or cell lines for incorporation of exogenous nucleic acid are provided in the present disclosure. In some embodiments, the method comprises introducing nucleic acid constructs to a composition of host cells and maintaining the composition of cells under conditions that permit incorporation and expression of exogenous nucleic acid in the host cells. Such conditions are well known and include, for example, conditions for introducing nucleic acid constructs to mammalian cells by transfection, transduction, and electroporation. In some embodiments of the present disclosure, mammalian cells (e.g., HEK293 cells) are transfected with plasmid DNA comprising at least one polynucleotide of interest and a selectable marker.

The selection of cells or cell lines for incorporation of exogenous nucleic acid depends on the type of selectable marker used. In some embodiments of the present disclosure, the selectable marker is a gene encoding an enzyme necessary for production of an essential nutrient. In some embodiments, selection requires incubating the cells or cell lines in media that lacks the essential nutrient. In particular embodiments, the essential nutrient is tyrosine.

In some embodiments, incorporation of exogenous nucleic acid is monitored by use of a fluorescent reporter protein (e.g., mCherry, EGFP). Fluorescence is measured by well known methods (e.g., flow cytometry).

Kits

In another aspect, components or embodiments described herein for the system are provided in a kit. For example, any of the plasmids, as well as the mammalian cells, related buffers, media, triggering agents, or other components related to cell culture and virion production can be provided, with optional components frozen and packaged as a kit, alone or along with separate containers of any of the other agents and optional instructions for use. In some embodiments, the kit may comprise culture vessels, vials, tubes, or the like.

### Numbered Embodiments #1

[1] A method of generating a recombinant eukaryotic host cell that can be selected to retain a first exogenous nucleic acid construct and a second exogenous nucleic acid construct with a single selective pressure, the method comprising:
introducing into a host cell:
a first nucleic acid construct comprising:
   a first polynucleotide of interest; and
   a first portion of a selectable marker; and
a second nucleic acid construct comprising:
   a second polynucleotide of interest; and
   a second portion of the selectable marker;
wherein the first portion of the selectable marker encodes a nonfunctional first portion of a selectable protein and the second portion of the selectable marker encodes a nonfunctional second portion of a selectable protein; and
wherein upon application of the single selective pressure, the nonfunctional first and second portions of the selectable protein are capable of assembling in the cell to create a functional selectable protein.

[2] The method of embodiment 1, wherein the host cell is a mammalian cell.

[3] The method of embodiment 2, wherein the mammalian cell is a human embryonic kidney (HEK) cell, chinese hamster ovary (CHO) cell, HeLa cell, or a derivative thereof.

[4] The method of embodiment 3, wherein the HEK cell is an HEK293 cell.

[5] The method of any of the preceding embodiments, wherein the host cell is suspension-adapted.

[6] The method of any of the preceding embodiments, wherein the recombinant eukaryotic host cell is capable of virus production.

[7] The method of any of the preceding embodiments, wherein the first nucleic acid construct, the second nucleic acid construct, or both the first and second nucleic acid constructs become stably incorporated into the host cell genome.

[8] The method of any of the preceding embodiments, wherein the host cell is a viral production cell.

[9] The method of any of the preceding embodiments, wherein the first polynucleotide of interest encodes an adeno-associated virus (AAV) Rep protein, an AAV Cap protein, an adenoviral helper protein, a first payload, or any combination thereof.

[10] The method of any of the preceding embodiments, wherein the second polynucleotide of interest encodes an adeno-associated virus (AAV) Rep protein, an AAV Cap protein, an adenoviral helper protein, a second payload, or any combination thereof.

[11] The method of embodiment 9 or 10, wherein the first and/or second payload is a guide RNA or a tRNA.

[12] The method of embodiment 9 or 10, wherein the first and/or second payload encodes a protein.

[13] The method of embodiment 9 or 10, wherein the first and/or second payload comprises a gene for replacement gene therapy.

[14] The method of embodiment 9 or 10, wherein the first and/or second payload comprises a homology construct for homologous recombination.

[15] The method of any of the preceding embodiments, wherein the selectable marker does not confer resistance to an antibiotic or a toxin.

[16] The method of any of the preceding embodiments, wherein the single selective pressure is not an antibiotic or a toxin.

[17] The method of any of the preceding embodiments, wherein the selectable protein is a functional enzyme.

[18] The method of embodiment 17, wherein the functional enzyme is not endogenous to the host cell.

[19] The method of embodiment 17, wherein the functional enzyme is endogenous to the host cell.

[20] The method of embodiment 17, wherein the functional enzyme catalyzes a reaction that results in production of a molecule necessary for growth of the host cell, wherein the host cell is grown in media deficient for the molecule.

[21] The method of embodiment 20, wherein the functional enzyme catalyzes the conversion of an amino acid into the molecule necessary for growth of the host cell.

[22] The method of embodiment 20, wherein the enzyme is dihydrofolate reductase (DHFR), glutamine synthetase (GS), thymidylate synthase (TYMS), phenylalanine hydroxylase (PAH), or any combination thereof.

[23] The method of embodiment 22, wherein the enzyme is dihydrofolate reductase (DHFR).

[24] The method of embodiment 22, wherein the enzyme is glutamine synthetase (GS).

[25] The method of embodiment 22, wherein the enzyme is thymidylate synthase (TYMS).

[26] The method of embodiment 21 or 22, wherein the enzyme is phenylalanine hydroxylase (PAH) and the PAH catalyzes the conversion of phenylalanine to tyrosine.

[27] The method of embodiment 23, wherein the molecule necessary for growth of the host cell is hypoxanthine and/or thymidine.

[28] The method of embodiment 24, wherein the molecule necessary for growth of the host cell is glutamine.

[29] The method of embodiment 25, wherein the molecule necessary for growth of the host cell is thymidine.

[30] The method of embodiment 26, wherein the molecule necessary for growth of the host cell is tyrosine.

[31] The method of embodiment 26, wherein PAH catalyzes the conversion of phenylalanine to tyrosine in the presence of (6R)-5,6,7,8-tetrahydrobiopterin (BH4) or a BH4 precursor.

[32] The method of 31, wherein the BH4 precursor is 7,8-dihydrobiopterin (7,8-BH2).

[33] The method of any of the preceding embodiments, wherein the host cell is grown in a media deficient for a molecule necessary for growth of the host cell.

[34] The method of embodiment 33, wherein the molecule necessary for growth of the host cell is tyrosine.

[35] The method of any of the preceding embodiments, wherein the first portion of the selectable marker is fused to a coding sequence of an N-terminal fragment of a split intein.

[36] The method of any one of the preceding embodiments, wherein the second portion of the selectable marker is fused to a coding sequence of a C-terminal fragment of a split intein.

[37] The method of embodiment 35 or 36, wherein the split intein is derived from the Nostoc punctiforme (Npu) DnaE intein, the Synechocystis species, strain PCC6803 (Ssp) DnaE intein, or the consensus DnaE intein (Cfa).

[38] The method of any one of the preceding embodiments, wherein the nonfunctional first portion of a selectable protein and the nonfunctional second portion of a selectable protein are linked by a peptide bond at a split point in the functional selectable protein.

[39] The method of embodiment 38, wherein the split point is a cysteine or serine residue within the catalytic domain of the functional selectable protein.

[40] The method of embodiment 38 or 39, wherein the nonfunctional first portion of a selectable protein is the N-terminal fragment of the functional selectable protein.

[41] The method of any of embodiments 38-40, wherein the nonfunctional second portion of a selectable protein is the C-terminal fragment of the functional selectable protein.

[42] The method of embodiment 41, wherein the N-terminal residue of the nonfunctional second portion of a selectable protein is cysteine or serine.

[43] The method of embodiment 42, wherein the N-terminal residue is cysteine.

[44] The method of any embodiment 20 or 21, wherein activity of the functional enzyme is enabled by expression of a polypeptide encoded by the first or second nucleic acid constructs.

[45] The method of embodiment 41, wherein the functional enzyme is glutamine synthetase (GS), thymidylate synthase (TYMS), or phenylalanine hydroxylase (PAH).

[46] The method of embodiment 44 or 45, wherein the polypeptide is an enzyme that catalyzes production of a cofactor.

[47] The method of any one of embodiments 44-46, wherein the first or second nucleic acid construct further encodes GTP cyclohydrolase I (GTP-CH1).

[48] The method of embodiment 47, wherein the host cell expresses GTP-CH1.

[49] The method of embodiment 48, wherein expression of GTP-CH1 facilitates growth of the host cell in conjunction with the functional enzyme upon application of the single selective pressure.

[50] The method of embodiment 46, wherein the cofactor is (6R)-5,6,7,8-tetrahydrobiopterin (BH4).

[51] The method of any one of embodiments 1-45, further comprising growing the host cell in media comprising a cofactor.

[52] The method of embodiment 51, wherein the cofactor is (6R)-5,6,7,8-tetrahydrobiopterin (BH4).

[53] The method of embodiment 51, wherein the cofactor is a (6R)-5,6,7,8-tetrahydrobiopterin (BH4) precursor molecule.

[54] The method of embodiment 53, wherein the BH4 precursor molecule is 7,8-dihydobiopterin (7,8-BH2).

[55] The method of any one of the preceding embodiments, further comprising applying the single selective pressure.

[56] The method of embodiment 55, wherein the applying the single selective pressure comprises growing the host cell in media deficient in at least one nutrient.

[57] The method of embodiment 56, wherein the host cell is grown in media deficient in tyrosine.

[58] The method of any one of the preceding embodiments, further comprising applying a second selective pressure, wherein application of the second selective pressure selects for cells that highly express the first portion and second portion of the selectable marker.

[59] The method of embodiment 58, wherein the second selective pressure is the presence of an inhibitor.

[60] The method of embodiment 59, wherein the inhibitor inhibits activity of the functional enzyme.

[61] The method of any one of the preceding embodiments, wherein a virus particle produced by the recombinant eukaryotic host cell has an increased safety profile as compared to a virus particle produced by a method wherein the single selective pressure is an antibiotic.

[62] The method of any one of the preceding embodiments, wherein the method yields an increase in a number of clones integrated with the first and second polynucleotide of interest as compared to a method wherein the single selective pressure is an antibiotic or a method wherein two different selectable markers are used.

[63] A composition of plasmids for stably transfecting a eukaryotic host cell with two or more exogenous nucleic acid constructs that are capable of being retained in the cell with a single selective pressure, comprising:
a first plasmid comprising:
a first polynucleotide of interest; and
a first portion of a selectable marker; and
a second plasmid comprising:
a second polynucleotide of interest; and
a second portion of a selectable marker.

[64] The composition of embodiment 63, wherein the host cell is a mammalian cell.

[65] The composition of embodiment 64, wherein the mammalian cell is a human embryonic kidney (HEK) cell.

[66] The composition of any one of embodiments 63-65, wherein the first nucleic acid construct and/or the second nucleic acid construct become stably incorporated into the host cell genome.

[67] The composition of any one of embodiments 63-66, wherein the host cell is a viral production cell.

[68] The composition of any one of embodiments 63-67, wherein the first polynucleotide of interest encodes an adeno-associated virus (AAV) Rep protein, an AAV Cap protein, an adenoviral helper protein, a first payload, or any combination thereof.

[69] The composition of any one of embodiments 63-68, wherein the second polynucleotide of interest encodes an adeno-associated virus (AAV) Rep protein, an AAV Cap protein, an adenoviral helper protein, a second payload, or any combination thereof.

[70] The composition of any one of embodiments 63-69, wherein the selectable marker does not confer resistance to an antibiotic or a toxin.

[71] The composition of any one of embodiments 63-70, wherein the selectable marker comprises a nucleic acid sequence that encodes a functional enzyme.

[72] The composition of embodiment 71, wherein the functional enzyme is not endogenous to the host cell.

[73] The composition of embodiment 71 or 72, wherein the functional enzyme catalyzes a reaction that results in production of a molecule necessary for growth of the host cell, wherein the host cell is grown in media deficient for the molecule.

[74] The composition of embodiment 73, wherein the enzyme is dihydrofolate reductase (DHFR), glutamine synthetase (GS), thymidylate synthase (TYMS), phenylalanine hydroxylase (PAH), or any combination thereof.

[75] The composition of embodiment 74, wherein the enzyme is dihydrofolate reductase (DHFR).

[76] The composition of embodiment 74, wherein the enzyme is glutamine synthetase (GS).

[77] The method of embodiment 74, wherein the enzyme is thymidylate synthase (TYMS).

[78] The composition of embodiment 74, wherein the enzyme is phenylalanine hydroxylase (PAH).

[79] The composition of embodiment 75, wherein the molecule necessary for growth of the host cell is hypoxanthine and/or thymidine.

[80] The composition of embodiment 76, wherein the molecule necessary for growth of the host cell is glutamine.

[81] The composition of embodiment 77, wherein the molecule necessary for growth of the host cell is thymidine.

[82] The composition of embodiment 78, wherein the molecule necessary for growth of the host cell is tyrosine.

[83] The composition of any one of embodiments 63-82, wherein the first portion of the selectable marker encodes a nonfunctional first portion of a selectable protein and the second portion of the selectable marker encodes a nonfunctional second portion of a selectable protein.

[84] The composition of any one of embodiments 63-83, wherein the first portion of the selectable marker is fused to a coding sequence of an N-terminal fragment of a split intein.

[85] The composition of any one of embodiments 63-84, wherein the second portion of the selectable marker is fused to a coding sequence of a C-terminal fragment of a split intein.

[86] The composition of embodiment 84 or 85, wherein the split intein is derived from the Nostoc punctiforme (Npu) DnaE intein, the Synechocystis species, strain PCC6803 (Ssp) DnaE intein, or the consensus DnaE intein (Cfa).

[87] The composition of embodiment 83, wherein the nonfunctional first portion of a selectable protein and the nonfunctional second portion of a selectable protein are linked by a peptide bond at a split point in a functional selectable protein.

[88] The composition of embodiment 87, wherein the split point is a cysteine or serine residue within the catalytic domain of the functional selectable protein.

[89] The composition of embodiment 87 or 88, wherein the nonfunctional first portion of a selectable protein is the N-terminal fragment of the functional selectable protein.

[90] The composition of any one of embodiments 87-89, wherein the nonfunctional second portion of a selectable protein is the C-terminal fragment of the functional selectable protein.

[91] The composition of embodiment 90, wherein the N-terminal residue of the nonfunctional second portion of a selectable protein is cysteine or serine.

[92] The composition of embodiment 91, wherein the N-terminal residue is cysteine.

[93] The composition of embodiment 73 or 78, wherein activity of the functional enzyme is enhanced by expression of a polypeptide encoded by the first or second nucleic acid construct.

[94] The composition of embodiment 93, wherein the functional enzyme is phenylalanine hydroxylase (PAH).

[95] The composition of embodiment 93 or 94, wherein the polypeptide is an enzyme that catalyzes production of a cofactor.

[96] The composition of any one of embodiments 63-74, 78, or 82-95, wherein the first or second nucleic acid construct further encodes GTP cyclohydrolase I (GTP-CH1).

[97] The composition of embodiment 96, wherein the host cell overexpresses GTP-CH1.

[98] The composition of embodiment 97, wherein expression of GTP-CH1 facilitates growth of the host cell in conjunction with the functional enzyme upon application of the single selective pressure.

[99] The composition of embodiment 98, wherein the single selective pressure is tyrosine deficiency.

[100] The composition of embodiment 95, wherein the cofactor is tetrahydrobiopterin (BH4).

[101] The composition of any one of embodiments 63-74, 78, or 82-100, further comprising:

[102] media for growing the eukaryotic host cell, wherein the media comprises a cofactor.

[103] The composition of embodiment 101, wherein the cofactor is (6R)-5,6,7,8-tetrahydrobiopterin (BH4).

[104] The composition of embodiment 101, wherein the cofactor is a (6R)-5,6,7,8-tetrahydrobiopterin (BH4) precursor molecule.

[105] The composition of 103, wherein the BH4 precursor molecule is 7,8-dihydobiopterin (7,8-BH2).

[106] A eukaryotic cell or cell line, wherein:
the cell or cell line is selected to retain a first exogenous nucleic acid construct and a second exogenous nucleic acid construct with a single selective pressure;
the first nucleic acid construct comprises:
   a first polynucleotide of interest; and
   a first portion of a selectable marker;
the second nucleic acid construct comprises:
   a second polynucleotide of interest; and
   a second portion of a selectable marker;
wherein the first portion of the selectable marker encodes a nonfunctional first portion of a selectable protein and the second portion of the selectable marker encodes a nonfunctional second portion of a selectable protein;
survival of the cell or cell line under the single selective pressure requires expression of a functional selectable protein; and
the functional selectable protein is generated by protein splicing the nonfunctional first and second portions of the selectable protein.

[107] The cell or cell line of embodiment 105, wherein the cell or cell line is mammalian.

[108] The cell or cell line of embodiment 106, wherein the cell or cell line is human embryonic kidney (HEK).

[109] The cell or cell line of any of embodiments 105-107, wherein the first polynucleotide of interest encodes an adeno-associated virus (AAV) Rep protein, an AAV Cap protein, an adenoviral helper protein, a first payload, or any combination thereof.

[110] The cell or cell line of any of embodiments 105-108, wherein the second polynucleotide of interest encodes an adeno-associated virus (AAV) Rep protein, an AAV Cap protein, an adenoviral helper protein, a payload, or any combination thereof.

[111] The cell or cell line of any of embodiments 105-109, wherein the functional selectable protein does not confer resistance to an antibiotic or a toxin.

[112] The cell or cell line of any of embodiments 105-110, wherein the functional selectable protein is not endogenous to the cell or cell line.

[113] The cell or cell line of any of embodiments 105-111, wherein the functional selectable protein catalyzes a reaction that results in production of a molecule necessary for growth of the cells when the cells are grown in media deficient in the molecule.

[114] A method of selecting a cell for retention of at least two exogenous nucleic acid constructs, wherein:
a single selective pressure is used for selecting a cell for retention of the at least two nucleic acid constructs;
expression of a functional selectable protein is required for the cell to survive the selective pressure; and
the functional selectable protein is expressed following protein trans-splicing of nonfunctional polypeptide fragments, wherein the nonfunctional polypeptide fragments are encoded by at least two separate nucleic acid constructs.

[115] The method, composition, cell, or cell line of any one of embodiments 1-113, wherein a construct encoding for at least a portion of PAH comprises a sequence having at least 80% sequence identity to a portion of any one of SEQ ID NO: 1-SEQ ID NO: 9 or SEQ ID NO: 12-SEQ ID NO: 20.

[116] The method, composition, cell, or cell line of any one of embodiments 1-114, wherein a construct encoding for GTP-CH1 comprises a sequence having at least 80% sequence identity to a portion of any one of SEQ ID NO: 10 or SEQ ID NO: 12-SEQ ID NO: 20.

[117] The method, composition, cell, or cell line of any one of embodiments 1-115, wherein a construct encoding for at least a portion of glutamine synthetase (GS) comprises a sequence having at least 80% sequence identity to a portion of any one of SEQ ID NO: 23-SEQ ID NO: 33.

[118] The method, composition, cell, or cell line of any one of embodiments 1-115, wherein a construct encoding for at least a portion of thymidylate synthase (TYMS) comprises a sequence having at least 80% sequence identity to a portion of any one of SEQ ID NO: 34-SEQ ID NO: 42.

[119] The method, composition, cell, or cell line of any one of embodiments 1-115, wherein a construct encoding for a portion of an intein comprises a sequence having at least 80% sequence identity to a portion of any one of SEQ ID NO: 2-SEQ ID NO: 9, SEQ ID NO: 13-SEQ ID NO: 20, SEQ ID NO: 24-SEQ ID NO: 33, or SEQ ID NO: 35-SEQ ID NO: 42.

### Numbered Embodiments #2

1. A method of generating a cell that retains a first nucleic acid construct and a second nucleic acid construct upon application of a single selective pressure, the method comprising:
introducing into the cell:
a) a first nucleic acid construct comprising:
   i) a first polynucleotide sequence; and
   ii) a first portion of a selectable marker; and
b) a second nucleic acid construct comprising:
   i) a second polynucleotide sequence; and
   ii) a second portion of the selectable marker;
wherein the first portion of the selectable marker encodes a nonfunctional first portion of a selectable protein and the second portion of the selectable marker encodes a nonfunctional second portion of the selectable protein; and
thereby, upon application of the single selective pressure, the cell retains the first nucleic acid construct and the second nucleic acid construct.

2. The method of embodiment 1, wherein the first nucleic acid construct is a first exogenous nucleic acid construct.

3. The method of embodiment 1 or 2, wherein the second nucleic acid construct is a second exogenous nucleic acid construct.

4. The method of any one of embodiments 1-3, wherein the cell is a host cell.

5. The method of any one of embodiments 1-4, wherein the cell is a eukaryotic cell.

6. The method of any one of embodiments 1-5, wherein the cell is a eukaryotic host cell.

7. The method of any one of embodiments 1-6, wherein the cell is a recombinant eukaryotic host cell.

8. The method of any one of embodiments 1-7, wherein the cell is capable of assembling the first portion of the selectable marker and the second portion of the selectable maker into a functional selectable marker.

9. The method of any one of embodiments 1-8, wherein the cell survives the single selective pressure by assembling the first portion of the selectable marker and the second portion of the selectable maker into a functional selectable marker.

10. The method of any one of embodiments 1-9, wherein the cell is capable of assembling the nonfunctional first portion of the selectable protein and the nonfunctional second portion of the selectable protein into a functional selectable protein.

11. The method of any one of embodiments 1-10, wherein the cell survives the single selective pressure by assembling the nonfunctional first portion of the selectable protein and the nonfunctional second portion of the selectable protein into a functional selectable protein.

12. The method of any one of embodiments 1-11, wherein the cell is a mammalian cell.

13. The method of embodiment 2, wherein the cell is a human embryonic kidney (HEK) cell, chinese hamster ovary (CHO) cell, HeLa cell, or a derivative thereof.

14. The method of embodiment 3, wherein the HEK cell is an HEK293 cell.

15. The method of any of the preceding embodiments, wherein the cell is suspension-adapted.

16. The method of any of the preceding embodiments, wherein the cell is capable of virus production.

17. The method of any one of embodiments 1-16, wherein the cell is capable of virus production.

18. The method of any one of embodiments 1-17, wherein the cell is capable of adeno-associated virus (AAV) production.

19. The method of any of the preceding embodiments, wherein the first nucleic acid construct, the second nucleic acid construct, or both the first nucleic acid construct and the second nucleic acid construct become stably incorporated into the genome of the cell.

20. The method of any one of embodiments 1-19, wherein the first nucleic acid construct, the second nucleic acid construct, or both the first nucleic acid construct and second nucleic acid construct are stably maintained extrachromosomally in the cell.

21. The method of any one of embodiments 1-20, wherein the first nucleic acid construct is in a first plasmid or a first episome.

22. The method of any one of embodiments 1-21, wherein the second nucleic acid construct is in a second plasmid or a second episome.

23. The method of any one of embodiments 1-22, wherein the first plasmid or the first episome, the second plasmid or the second episome, or any combination thereof comprise an Epstein-Barr virus (EBV) sequence; optionally, wherein the EBV sequence comprises one or more of oriP and/or EBNA1.

24. The method of any of the preceding embodiments, wherein the cell is a viral production cell.

25. The method of any one of embodiments 1-24, wherein the first polynucleotide sequence encodes a first polynucleotide of interest.

26. The method of any one of embodiments 1-25, wherein the second polynucleotide encodes a second polynucleotide of interest.

27. The method of any of the preceding embodiments, wherein the first polynucleotide sequence encodes one or more of an adeno-associated virus (AAV) Rep protein, an AAV Cap protein, an adenoviral helper protein, a first payload, or any combination thereof.

28. The method of any of the preceding embodiments, wherein the second polynucleotide of sequence encodes one or more of an adeno-associated virus (AAV) Rep protein, an AAV Cap protein, an adenoviral helper protein, a second payload, or any combination thereof.

29. The method of any of the preceding embodiments, wherein the first polynucleotide of interest encodes one or more of adeno-associated virus (AAV) Rep proteins, AAV Cap proteins, adenoviral helper proteins, a first payload, or any combination thereof.

30. The method of any of the preceding embodiments, wherein the second polynucleotide of interest encodes one or more of an adeno-associated virus (AAV) Rep protein, an AAV Cap protein, an adenoviral helper protein, a second payload, or any combination thereof.

31. The method of any one of embodiments 27-30, wherein the AAV Rep proteins comprises one or more of Rep78, Rep68, Rep52, Rep40, or any combination thereof.

32. The method of any one of embodiments 27-31, wherein the AAV Cap protein comprises one or more of VP1, VP2, VP3, or any combination thereof.

33. The method of any one of embodiments 27-32, wherein the AAV helper proteins comprises one or more of E1A, E1B, E2A, E4, or any combination thereof.

34. The method of any one of embodiments 1-33, wherein the construct further comprises a sequence encoding VA RNA.

35. The method of embodiment 34, wherein the sequence encoding VA RNA encodes for a mutant VA RNA; optionally, wherein the mutant VA RNA comprises a G16A mutation, a G60A mutation, or a combination thereof.

36. The method of any one of embodiments 1-35, wherein the first polynucleotide sequence or the second polynucleotide sequence comprises at least 80%, 85%, 90%, 95%, 98%, 99%, or 100% sequence identity to SEQ ID NO: 47.

37. The method any one of embodiments 1-36, wherein the first polynucleotide sequence or the second polynucleotide sequence comprises at least 80%, 85%, 90%, 95%, 98%, 99%, or 100% sequence identity to SEQ ID NO: 48.

38. The method any one of embodiments 1-37, wherein the first polynucleotide sequence or the second polynucleotide sequence comprises at least 80%, 85%, 90%, 95%, 98%, 99%, or 100% sequence identity to SEQ ID NO: 49.

39. The method of any one of embodiments 1-38, wherein the first and/or second payload encodes a guide RNA or a tRNA; optionally, wherein the tRNA is a suppressor tRNA.

40. The method of any one of embodiments 1-38, wherein the first and/or second payload encodes a protein.

41. The method of any one of embodiments 1-38, wherein the first and/or second payload encodes a gene; optionally, wherein the gene is for replacement gene therapy or is a transgene.

42. The method of any one of embodiments 1-38, wherein the first and/or second payload comprises a homology construct for homologous recombination.

43. The method of any one of embodiments 1-38, wherein the first payload and/or the second payload is flanked by a 5′ AAV inverted terminal repeat (5′ ITR) and a 3′ AAV inverted terminal repeat (3′ ITR).

44. The method of any of the preceding embodiments, wherein the selectable marker or selectable protein does not confer resistance to an antibiotic or a toxin.

45. The method of any of the preceding embodiments, wherein the single selective pressure is not an antibiotic or a toxin.

46. The method of any one of embodiments 1-45, wherein the selectable marker is a selectable protein.

47. The method of any of the preceding embodiments, wherein the selectable protein is a functional enzyme.

48. The method of embodiment 47, wherein the functional enzyme is not endogenous to the cell.

49. The method of embodiment 47, wherein the functional enzyme is endogenous to a genome of the cell.

50. The method of embodiment 48, wherein the functional enzyme that is endogenous to the genome of the cell is genetically altered to a nonfunctional enzyme.

51. The method of any one of embodiments 47-50, wherein the functional enzyme catalyzes a reaction that results in production of a molecule necessary for growth of the cell, wherein the cell is grown in media deficient for the molecule.

52. The method of embodiment 51, wherein the functional enzyme catalyzes the conversion of an amino acid into the molecule necessary for growth of the cell.

53. The method of embodiment 52, wherein the enzyme is dihydrofolate reductase (DHFR), glutamine synthetase (GS), thymidylate synthase (TYMS), phenylalanine hydroxylase (PAH), or any combination thereof.

54. The method of embodiment 53, wherein the enzyme is dihydrofolate reductase (DHFR).

55. The method of embodiment 53, wherein the enzyme is glutamine synthetase (GS).

56. The method of embodiment 53, wherein the enzyme is thymidylate synthase (TYMS).

57. The method of embodiment 53, wherein the enzyme is phenylalanine hydroxylase (PAH) and the PAH catalyzes the conversion of phenylalanine to tyrosine.

58. The method of embodiment 56, wherein the molecule necessary for growth of the cell is hypoxanthine and/or thymidine.

59. The method of embodiment 55, wherein the molecule necessary for growth of the cell is glutamine 60. The method of embodiment 56, wherein the molecule necessary for growth of the cell is thymidine.

61. The method of embodiment 57 wherein the molecule necessary for growth of the cell is tyrosine.

62. The method of embodiment 61, wherein PAH catalyzes the conversion of phenylalanine to tyrosine in the presence of (6R)-5,6,7,8-tetrahydrobiopterin (BH4) or a BH4 precursor.

63. The method of 62, wherein the BH4 precursor is 7,8-dihydrobiopterin (7,8-BH2).

64. The method of any of the preceding embodiments, wherein the cell is grown in a media deficient for a molecule necessary for growth of the cell.

65. The method of embodiment 64, wherein the molecule necessary for growth of the cell is tyrosine.

66. The method of any of the preceding embodiments, wherein the first portion of the selectable marker is fused to a coding sequence of an N-terminal fragment of a split intein.

67. The method of any of the preceding embodiments, wherein the first portion of the selectable marker comprises a sequence encoding an N-terminal fragment of the selectable protein fused in-frame to a sequence encoding an N-terminal fragment of an intein.

68. The method of any of the preceding embodiments, wherein the first portion of the selectable marker comprises a sequence encoding the nonfunctional first portion of the selectable protein fused in-frame to a sequence encoding an N-terminal fragment of an intein.

69. The method of any one of the preceding embodiments, wherein the second portion of the selectable marker is fused to a coding sequence of a C-terminal fragment of a split intein.

70. The method of 69, wherein the second portion of the selectable marker comprises a sequence encoding a C-terminal fragment of an intein fused in-frame to a sequence encoding a C-terminal fragment of the selectable protein.

71. The method of 69, wherein the second portion of the selectable marker comprises a sequence encoding an N-terminal fragment of an intein fused in-frame to a sequence encoding the nonfunctional second portion of the selectable protein.

72. The method of any one of embodiments 66-71, wherein the sequence encoding the N-terminal fragment of the intein comprises at least 80%, 85%, 90%, 95%, 98%, 99%, or 100% sequence identity to SEQ ID NO: 53.

73. The method of any one of embodiments 66-72, wherein the sequence encoding the C-terminal fragment of the intein comprises at least 80%, 85%, 90%, 95%, 98%, 99%, or 100% sequence identity to SEQ ID NO: 54.

74. The method of embodiment 72 or 73, wherein the intein is derived from the Nostoc punctiforme (Npu) DnaE intein, the Synechocystis species, strain PCC6803 (Ssp) DnaE intein, or the consensus DnaE intein (Cfa).

75. The method of any one of the preceding embodiments, wherein the nonfunctional first portion of the selectable protein and the nonfunctional second portion of the selectable protein are linked by a peptide bond at a split point in the selectable protein.

76. The method of embodiment 75, wherein the split point is a cysteine or serine residue within the catalytic domain of the selectable protein.

77. The method of embodiment 75 or 76, wherein the nonfunctional first portion of a selectable protein is an N-terminal fragment of the selectable protein.

78. The method of any of embodiments 76-77, wherein the nonfunctional second portion of the selectable protein is a C-terminal fragment of the selectable protein.

79. The method of embodiment 78, wherein the C-terminal residue of the nonfunctional first portion of a selectable protein is cysteine or serine.

80. The method of embodiment 79, wherein the C-terminal residue is cysteine.

81. The method of any one of embodiments 48-80, wherein activity of the functional enzyme is enabled by expression of a first polypeptide encoded by the first nucleic acid construct and by expression of a second polypeptide encoded by the second nucleic acid construct.

82. The method of embodiment 81, wherein the functional enzyme is dihydrofolate reductase (DHFR), glutamine synthetase (GS), thymidylate synthase (TYMS), or phenylalanine hydroxylase (PAH).

83. The method of embodiment 81 or 82, wherein the functional enzyme catalyzes production of a cofactor.

84. The method of any one of embodiments 81-83, wherein the first nucleic acid construct or second nucleic acid construct further encodes a helper enzyme that facilitates production of a molecule required for growth of the cell.

85. The method of 84, wherein the helper enzyme is GTP cyclohydrolase I (GTP-CH1).

86. The method of embodiment 81 or 83, wherein the cell expresses GTP-CH1.

87. The method of embodiment 85 or 86, wherein expression of GTP-CH1 facilitates growth of the cell in conjunction with the functional enzyme upon application of the single selective pressure.

88. The method of embodiment 85 or 86, wherein GTP-CH1 produced the cofactor (6R)-5,6,7,8-tetrahydrobiopterin (BH4).

89. The method of any one of embodiments 1-88, further comprising growing the cell in media comprising a cofactor.

90. The method of embodiment 89 wherein the cofactor is (6R)-5,6,7,8-tetrahydrobiopterin (BH4).

91. The method of embodiment 89, wherein the cofactor is a (6R)-5,6,7,8-tetrahydrobiopterin (BH4) precursor molecule.

92. The method of embodiment 89, wherein the BH4 precursor molecule is 7,8-dihydobiopterin (7,8-BH2).

93. The method of any one of the preceding embodiments, further comprising applying the single selective pressure.

94. The method of embodiment 93, wherein the applying the single selective pressure comprises growing the cell in media deficient in at least one nutrient.

95. The method of embodiment 94, wherein the cell is grown in media deficient in tyrosine, hypoxanthine, thymidine, glutamine, or any combination thereof.

96. The method of any one of embodiments 93-95, wherein the single selective pressure further selects for the cell comprising a high copy number of the first nucleic acid construct and the second nucleic acid construct.

97. The method of any one of embodiments 1-96, wherein the selectable protein comprises a mutation resulting in decreased enzymatic activity compared to a selectable protein lacking the mutation.

98. The method of embodiment 97, wherein the selectable protein is GS that comprises a R324C, R324S, or R341C mutation as compared to the selectable protein lacking the mutation that comprises SEQ ID NO: 23.

99. The method of any one of embodiments 96-98, wherein expression of the first portion of the selectable marker is selectable protein is reduced.

100. The method of embodiment 99, wherein expression of the first portion of the selectable marker is selectable protein is driven by an attenuated promoter.

101. The method of embodiment any one of embodiments 96-100, wherein expression of the second portion of the selectable marker is selectable protein is driven by an attenuated promoter.

102. The method of embodiment 101, wherein the attenuated promoter comprises an attenuated EF1alpha promoter; optionally, wherein the attenuated EF1alpha promoter has a sequence that is SEQ ID NO: 43.

103. The method of any one of the preceding embodiments, further comprising applying a second selective pressure, wherein application of the second selective pressure selects for cells that highly express the first portion of the selectable marker and the second portion of the selectable marker.

104. The method of embodiment 103, wherein the second selective pressure is the presence of an inhibitor.

105. The method of embodiment 104, wherein the inhibitor inhibits activity of the functional enzyme.

106. The method of embodiment 105, wherein the functional enzyme is GS and the inhibitor comprises Methionine Sulfoximine (MSX).

107. The method of embodiment 105, wherein the functional enzyme is DHFR and the inhibitor comprises methotrexate, ochratoxin A, alpha-methyl-tyrosine, alpha-methyl-phenylalanine, beta-2-thienyl-DL-alanine, or fenclonine.

108. The method of any one of the preceding embodiments, wherein the second nucleic acid construct comprises:
a first promoter and the second polynucleotide of interest, wherein the first promoter is operably linked to the second polynucleotide of interest;
a second promoter and the sequence encoding the C-terminal fragment of the intein fused in-frame to the sequence encoding the C-terminal fragment of the functional selectable protein, wherein the second promoter is operably linked to the sequence encoding the C-terminal fragments of the intein and the functional selectable protein, and
wherein the 3′ end of the coding strand of the second polynucleotide of interest is adjacent to the 3′ end of the coding strand for the C-terminal fragment of the intein fused in-frame to the sequence encoding the C-terminal fragment of the functional selectable protein such that a direction of transcription of the second polynucleotide of interest and a direction of transcription of the C-terminal fragment of the intein fused in-frame to the sequence encoding a C-terminal fragment of the functional selectable protein are towards each other.

109. The method of any one of the preceding embodiments, wherein the first nucleic acid construct comprises:
a first promoter and the first polynucleotide of interest, wherein the first promoter is operably linked to the first polynucleotide of interest;
a second promoter and the sequence encoding the N-terminal fragment of the functional selectable protein fused in-frame to the sequence encoding the N-terminal fragment of the intein, wherein the second promoter is operably linked to the sequence encoding the N-terminal fragments of the functional selectable protein and the intein,
wherein the 5′ end of the coding strand for the first polynucleotide of interest is adjacent to the 5′ end of the coding strand for the N-terminal fragment of the functional selectable protein and the N-terminal fragment of the intein such that a direction of transcription of the first polynucleotide of interest and a direction of transcription of the N-terminal fragment of the functional selectable protein and the N-terminal fragment of the intein proceeds away from the 5′ end of the respective sequences.

110. The method of any one of the preceding embodiments, wherein a virus particle produced by the cell has an increased safety profile as compared to a virus particle produced by a method wherein the single selective pressure is an antibiotic.

111. A method of generating cells that retain a first nucleic acid construct and a second nucleic acid construct upon application of a single selective pressure, the method comprising:
introducing into the cells:
a first nucleic acid construct and a second nucleic acid construct as set forth in any one of embodiments 1-110; and
thereby, upon application of the single selective pressure, the cells retain the first nucleic acid construct and the second nucleic acid construct.

112. The method of embodiment 111, wherein the cell is a mammalian cell and optionally wherein the mammalian cell is a human embryonic kidney (HEK) cell, Chinese hamster ovary (CHO) cell, or HeLa cell, and optionally wherein the host cell is suspension-adapted.

113. The method of any one of the preceding embodiments, wherein the method yields an increase in a number of the cells integrated with the first nucleic acid construct and second nucleic acid construct as compared to a method wherein the single selective pressure is an antibiotic or a method wherein two different selectable markers are used.

114. A composition of plasmids for transfecting a host cell with two or more exogenous nucleic acid constructs that are capable of being retained in the cell with a single selective pressure, comprising:
a) a first plasmid comprising:
   i) a first polynucleotide of interest; and
   ii) a first portion of a selectable marker; and
b) a second plasmid comprising:
   i) a second polynucleotide of interest; and
   ii) a second portion of a selectable marker,
wherein the first portion of the selectable marker encodes a nonfunctional first portion of a selectable protein and the second portion of the selectable marker encodes a nonfunctional second portion of the selectable protein.

115. The composition of embodiment 114, wherein the host cell is a mammalian cell.

116. The composition of embodiment 115, wherein the mammalian cell is a human embryonic kidney (HEK) cell.

117. The composition of any one of embodiments 114-116, wherein the first nucleic acid construct and/or the second nucleic acid construct become stably incorporated into the host cell genome.

118. The composition of any one of embodiments 114-117, wherein the host cell is a viral production cell.

119. The composition of any one of embodiments 114-118, wherein the first polynucleotide of interest encodes an adeno-associated virus (AAV) Rep protein, an AAV Cap protein, an adenoviral helper protein, a first payload, or any combination thereof.

120. The composition of any one of embodiments 114-119, wherein the second polynucleotide of interest encodes an adeno-associated virus (AAV) Rep protein, an AAV Cap protein, an adenoviral helper protein, a second payload, or any combination thereof.

121. The composition of any one of embodiments 114-120, wherein the selectable marker does not confer resistance to an antibiotic or a toxin.

122. The composition of any one of embodiments 114-121, wherein the selectable marker comprises a nucleic acid sequence that encodes a functional enzyme.

123. The composition of embodiment 122, wherein the functional enzyme is not endogenous to the host cell.

124. The composition of embodiment 122 or 123, wherein the functional enzyme catalyzes a reaction that results in production of a molecule necessary for growth of the host cell, wherein the host cell is grown in media deficient for the molecule.

125. The composition of embodiment 124, wherein the enzyme is dihydrofolate reductase (DHFR), glutamine synthetase (GS), thymidylate synthase (TYMS), phenylalanine hydroxylase (PAH), or any combination thereof.

126. The composition of embodiment 125, wherein the enzyme is dihydrofolate reductase (DHFR).

127. The composition of embodiment 125, wherein the enzyme is glutamine synthetase (GS).

128. The method of embodiment 125, wherein the enzyme is thymidylate synthase (TYMS).

129. The composition of embodiment 125, wherein the enzyme is phenylalanine hydroxylase (PAH).

130. The composition of embodiment 126, wherein the molecule necessary for growth of the cell is hypoxanthine and/or thymidine.

131. The composition of embodiment 127, wherein the molecule necessary for growth of the cell is glutamine.

132. The composition of embodiment 128, wherein the molecule necessary for growth of the cell is thymidine.

133. The composition of embodiment 129, wherein the molecule necessary for growth of the cell is tyrosine.

134. The composition of any one of embodiments 114-133, wherein the first portion of the selectable marker encodes a nonfunctional first portion of a selectable protein and the second portion of the selectable marker encodes a nonfunctional second portion of a selectable protein.

135. The composition of any one of embodiments 114-134, wherein the first portion of the selectable marker is fused to a coding sequence of an N-terminal fragment of an intein.

136. The composition of any one of embodiments 114-135, wherein the second portion of the selectable marker is fused to a coding sequence of a C-terminal fragment of an intein.

137. The composition of embodiment 135 or 136, wherein the intein is derived from the Nostoc punctiforme (Npu) DnaE intein, the Synechocystis species, strain PCC6803 (Ssp) DnaE intein, or the consensus DnaE intein (Cfa).

138. The composition of embodiment 137, wherein in a functional selectable protein, the nonfunctional first portion of a selectable protein and the nonfunctional second portion of a selectable protein are linked by a peptide bond at a split point in the functional selectable protein.

139. The composition of embodiment 138, wherein the split point is a cysteine or serine residue within the catalytic domain of the functional selectable protein.

140. The composition of embodiment 138 or 139, wherein the nonfunctional first portion of a selectable protein is the N-terminal fragment of the functional selectable protein.

141. The composition of any one of embodiments 138-140, wherein the nonfunctional second portion of a selectable protein is the C-terminal fragment of the functional selectable protein.

142. The composition of embodiment 141, wherein the N-terminal residue of the nonfunctional second portion of a selectable protein is cysteine or serine.

143. The composition of embodiment 142, wherein the N-terminal residue is cysteine.

144. The composition of any one of embodiments 122-143, wherein activity of the functional enzyme is enhanced by expression of a polypeptide encoded by the first or second nucleic acid construct.

145. The composition of embodiment 144, wherein the functional enzyme is phenylalanine hydroxylase (PAH).

146. The composition of embodiment 144 or 145, wherein the polypeptide is an enzyme that catalyzes production of a cofactor required for production of the molecule necessary for survival of the host cell.

147. The composition of any one of embodiments 114-125, 129, or 134-146, wherein the first or second nucleic acid construct further encodes GTP cyclohydrolase I (GTP-CH1).

148. The composition of embodiment 147, wherein the cell overexpresses GTP-CH1.

149. The composition of embodiment 148, wherein expression of GTP-CH1 facilitates survival of the host cell in conjunction with the functional enzyme upon application of the single selective pressure.

150. The composition of embodiment 149, wherein the single selective pressure is tyrosine deficiency.

151. The composition of embodiment 146, wherein the cofactor is tetrahydrobiopterin (BH4).

152. The composition of any one of embodiments 114-125, 129, or 134-151, further comprising:

c) media for growing the eukaryotic host cell, wherein the media comprises a cofactor.

153. The composition of embodiment 152, wherein the cofactor is (6R)-5,6,7,8-tetrahydrobiopterin (BH4).

154. The composition of embodiment 152, wherein the cofactor is a (6R)-5,6,7,8-tetrahydrobiopterin (BH4) precursor molecule.

155. The composition of 154, wherein the BH4 precursor molecule is 7,8-dihydobiopterin (7,8-BH2).

156. The composition of any one of embodiments 114-155, wherein the second nucleic acid construct comprises:
a first promoter and the second polynucleotide of interest, wherein the first promoter is operably linked to the second polynucleotide of interest;
a second promoter and the sequence encoding the C-terminal fragment of the intein fused in-frame to the sequence encoding the C-terminal fragment of the functional selectable protein, wherein the second promoter is operably linked to the sequence encoding the C-terminal fragments of the intein and the functional selectable protein, and
wherein the 3′ end of the coding strand of the second polynucleotide of interest is adjacent to the 3′ end of the coding strand for the C-terminal fragment of the intein fused in-frame to the sequence encoding the C-terminal fragment of the functional selectable protein such that a direction of transcription of the second polynucleotide of interest and a direction of transcription of the C-terminal fragment of the intein fused in-frame to the sequence encoding a C-terminal fragment of the functional selectable protein are towards each other.

157. The composition of any one of embodiments 114-156, wherein the first nucleic acid construct comprises:
a first promoter and the first polynucleotide of interest, wherein the first promoter is operably linked to the first polynucleotide of interest;
a second promoter and the sequence encoding the N-terminal fragment of the functional selectable protein fused in-frame to the sequence encoding the N-terminal fragment of the intein, wherein the second promoter is operably linked to the sequence encoding the N-terminal fragments of the functional selectable protein and the intein,
wherein the 5′ end of the coding strand for the first polynucleotide of interest is adjacent to the 5′ end of the coding strand for the N-terminal fragment of the functional selectable protein and the N-terminal fragment of the intein such that a direction of transcription of the first polynucleotide of interest and a direction of transcription of the N-terminal fragment of the functional selectable protein and the N-terminal fragment of the intein proceeds away from the 5′ end of the respective sequences.

158. A eukaryotic cell or cell line, wherein:
a) the cell or cell line is selected to retain a first exogenous nucleic acid construct and a second exogenous nucleic acid construct with a single selective pressure;
b) the first nucleic acid construct comprises:
   i) a first polynucleotide of interest as set forth in any one of embodiments 114-157; and
   ii) a first portion of a selectable marker as set forth in any one of embodiments 114-157;
c) the second nucleic acid construct comprises:
   i) a second polynucleotide of interest as set forth in any one of embodiments 114-157; and
   ii) a second portion of a selectable marker as set forth in any one of embodiments 114-157;
wherein the first portion of the selectable marker encodes a nonfunctional first portion of a selectable protein and the second portion of the selectable marker encodes a nonfunctional second portion of a selectable protein;
d) survival of the cell or cell line under the single selective pressure requires expression of a functional selectable protein; and
e) the functional selectable protein is generated by protein splicing the nonfunctional first and second portions of the selectable protein.

159. The cell or cell line of embodiment 158, wherein the cell or cell line is mammalian.

160. The cell or cell line of embodiment 159, wherein the cell or cell line is human embryonic kidney (HEK).

161. The cell or cell line of any of embodiments 158-160, wherein the first polynucleotide of interest encodes an adeno-associated virus (AAV) Rep protein, an AAV Cap protein, an adenoviral helper protein, a first payload, or any combination thereof.

162. The cell or cell line of any of embodiments 158-161, wherein the second polynucleotide of interest encodes an adeno-associated virus (AAV) Rep protein, an AAV Cap protein, an adenoviral helper protein, a payload, or any combination thereof.

163. The cell or cell line of any of embodiments 158-162, wherein the functional selectable protein does not confer resistance to an antibiotic or a toxin.

164. The cell or cell line of any of embodiments 158-163, wherein the functional selectable protein is not endogenous to the cell or cell line.

165. The cell or cell line of any of embodiments 158-164, wherein the functional selectable protein catalyzes a reaction that results in production of a molecule necessary for survival of the cells when the cells are grown in media deficient in the molecule.

166. A method of selecting a cell for retention of at least two exogenous nucleic acid constructs, wherein:
a single selective pressure is used for selecting a cell for retention of the at least two nucleic acid constructs;
expression of a functional selectable protein is required for the cell to survive the selective pressure; and
the functional selectable protein is expressed following protein trans-splicing of nonfunctional polypeptide fragments, wherein the nonfunctional polypeptide fragments are encoded by at least two separate nucleic acid constructs,
wherein a first nucleic acid construct of the at least two separate nucleic acid constructs comprises:
i) a first polynucleotide of interest as set forth in any one of embodiments 114-157; and
   ii) a first portion of a selectable marker as set forth in any one of embodiments 114-157;
a second nucleic acid construct of the at least two separate nucleic acid constructs comprises:
   i) a second polynucleotide of interest as set forth in any one of embodiments 114-157; and
   ii) a second portion of a selectable marker as set forth in any one of embodiments 114-157.

167. The method, composition, cell, or cell line of any one of embodiments 1-166, wherein a construct encoding for at least a portion of PAH comprises a sequence having at least 80% sequence identity to a portion of any one of SEQ ID NO: 1-SEQ ID NO: 9 or SEQ ID NO: 12-SEQ ID NO: 20.

168. The method, composition, cell, or cell line of any one of embodiments 1-167, wherein a construct encoding for GTP-CH1 comprises a sequence having at least 80% sequence identity to a portion of any one of SEQ ID NO: 10 or SEQ ID NO: 12-SEQ ID NO: 20.

169. The method, composition, cell, or cell line of any one of embodiments 1-166, wherein a construct encoding for at least a portion of glutamine synthetase (GS) comprises a sequence having at least 80% sequence identity to a portion of any one of SEQ ID NO: 23-SEQ ID NO: 33.

170. The method, composition, cell, or cell line of any one of embodiments 1-166, wherein a construct encoding for at least a portion of thymidylate synthase (TYMS) comprises a sequence having at least 80% sequence identity to a portion of any one of SEQ ID NO: 34-SEQ ID NO: 42.

180. The method, composition, cell, or cell line of any one of embodiments 1-170, wherein a construct encoding for a portion of an intein comprises a sequence having at least 80% sequence identity to a portion of any one of SEQ ID NO: 2-SEQ ID NO: 9, SEQ ID NO: 13-SEQ ID NO: 20, SEQ ID NO: 24-SEQ ID NO: 33, or SEQ ID NO: 35-SEQ ID NO: 42.

181. A method for producing a plurality of recombinant adeno-associated virus (rAAV) virions, the method comprising:
culturing a cell comprising the composition of any one of embodiments 119-157 or
culturing a cell of any one of embodiments 161-165,
under conditions sufficient for production of the rAAV.

182. The method of embodiment 181, wherein the first polynucleotide of interest
culturing comprises culturing the recombinant eukaryotic host cell or cell line in a culture medium deficient in a molecule required for growth of the recombinant eukaryotic host cell or cell line.

183. The method of embodiment 181 or 182, wherein the expression of one or more of an AAV Rep, an AAV Cap protein, an adenoviral helper protein, a first payload, and second payload, is inducible.

184. The method of any one of embodiments 181-183, wherein the first polynucleotide of interest encodes an adeno-associated virus (AAV) Rep protein and an AAV Cap protein, the second polynucleotide of interest encodes a payload, and the recombinant eukaryotic host cell or cell line further comprises a nucleic acid sequence encoding one or more adenoviral helper proteins.

185. The method of any one of embodiments 181-183, the first polynucleotide of interest encodes a first payload,
the second polynucleotide of interest encodes AAV Rep proteins and AAV Cap proteins, and
the functional selectable protein is a first functional selectable protein,
and the cell further comprises a nucleic acid construct comprising a polynucleotide sequence encoding a second functional selectable protein and one or more of AAV helper proteins and/or one or more VA RNA.

186. The method of any one of embodiments 181-183, wherein
the first polynucleotide of interest encodes AAV Rep proteins and AAV Cap proteins,
the second polynucleotide of interest encodes a first payload, and
the functional selectable protein is a first functional selectable protein,
and the cell further comprises a nucleic acid construct comprising a polynucleotide sequence encoding a second functional selectable protein and one or more of AAV helper proteins and/or one or more VA RNA.

187. The method of any one of embodiments 181-183, wherein
the first polynucleotide of interest encodes AAV Rep proteins and AAV Cap proteins,
the second polynucleotide of interest encodes one or more of AAV helper proteins and/or one or more VA RNA, and
the functional selectable protein is a first functional selectable protein,
and the cell further comprises a nucleic acid construct comprising a polynucleotide sequence encoding a second functional selectable protein and a first payload.

188. The method of any one of embodiments 181-183, wherein
the first polynucleotide of interest encodes a first payload,
the second polynucleotide of interest encodes one or more of AAV helper proteins and/or one or more VA RNA, and
the functional selectable protein is a first functional selectable protein,
and the cell further comprises a nucleic acid construct comprising a polynucleotide sequence encoding a second functional selectable protein and AAV Rep proteins and AAV Cap proteins.

189. The method of any one of embodiments 181-188, wherein the AAV Rep proteins comprise one or more of Rep78, Rep68, Rep52, Rep40, or any combination thereof.

190. The method of any one of embodiments 181-189, wherein the AAV Cap proteins one or more of VP1, VP2, VP3, or any combination thereof.

191. The method of any one of embodiments 181-190, wherein the AAV helper proteins comprise one or more of E1A, E1B, E2A, E4, or any combination thereof.

192. The method of any one of embodiments 181-191, wherein the sequence encoding VA RNA encodes for a mutant VA RNA; optionally, wherein the mutant VA RNA comprises a G16A mutation, a G60A mutation, or a combination thereof.

193. The method of any one of embodiments 181-192, wherein the first functional selectable protein is as set forth in any one of embodiments 125-129 or embodiments 167-170 and the second functional selectable protein is different from the first functional selectable protein.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1: Generating Plasmids with Split Selectable Markers

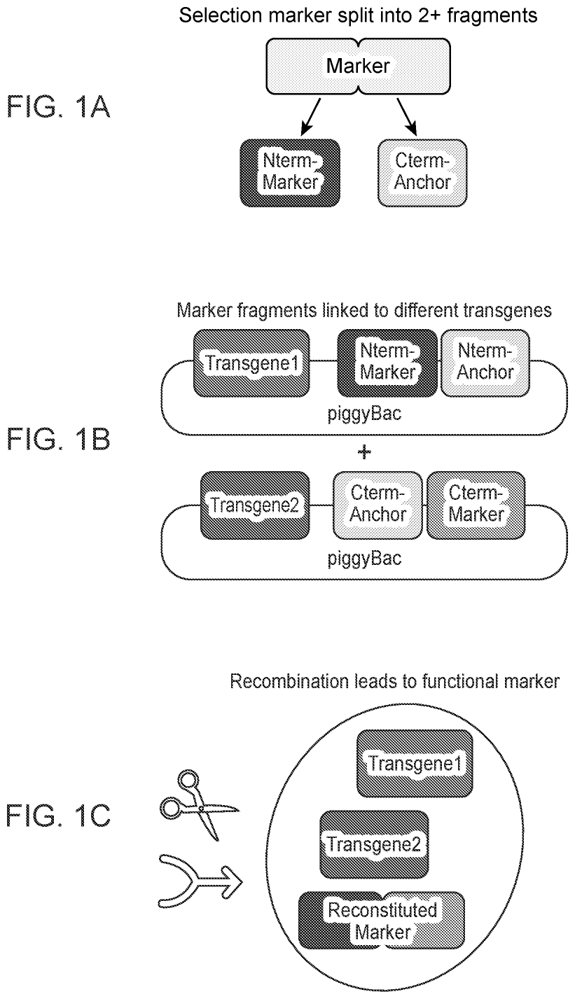

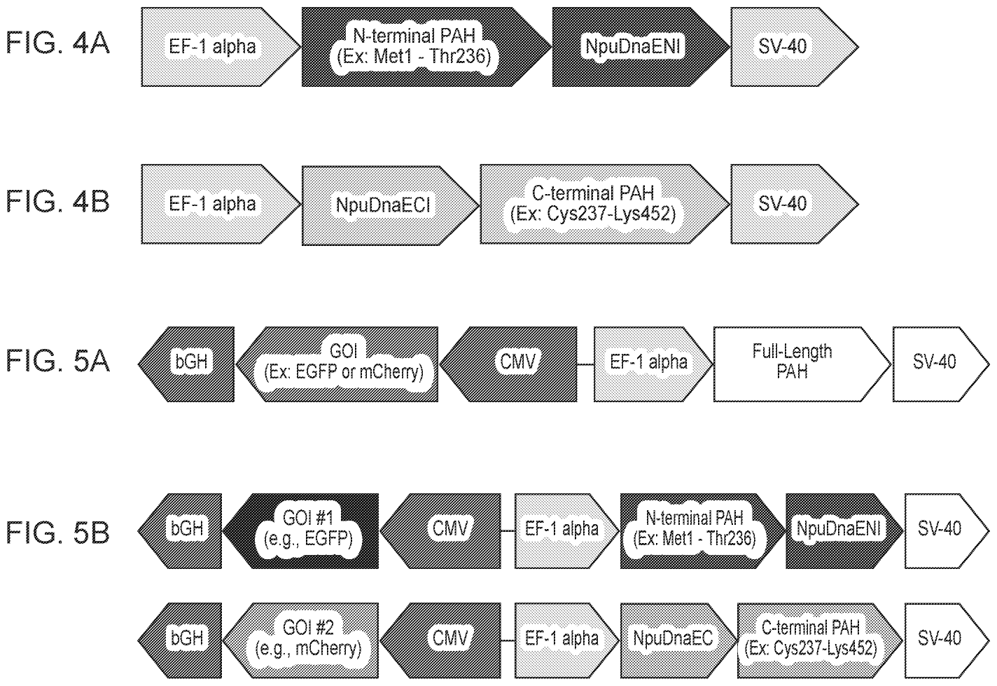

In order to generate cells that can be selected to retain at least two exogenous polynucleotides using only a single selective pressure, a selectable marker gene encoding the enzyme phenylalanine hydroxylase (PAH, SEQ ID NO: 1) was split into two fragments (FIG. 1A). The N-terminal fragment, encoding the N-terminal portion of PAH, was cloned into a piggyBac transposon plasmid in frame with the N-terminal fragment of the Nostoc punctiforme (Npu) split DnaE intein (NpuDnaE) (FIG. 1B and FIG. 4A). The C-terminal fragment, encoding the C-terminal portion of PAH, was cloned into a piggyBac transposon plasmid in frame with the C-terminal fragment of NpuDnaE (FIG. 1B and FIG. 4B).

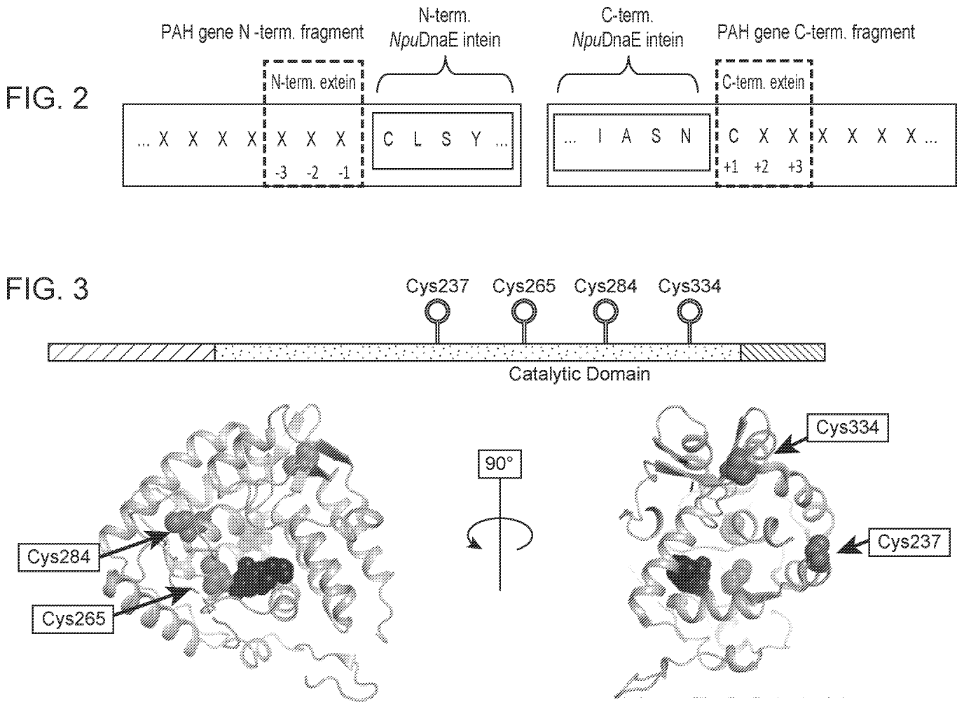

Previously described techniques were used to identify appropriate split points in the PAH-encoding gene and, accordingly, generate N-terminal and C-terminal gene fragments. See, e.g., Cheriyan et al., J Biol Chem 288:6202-6211 (2013); Stevens et al., Proc Natl Acad Sci 114: 8538-8543 (2017); and Jillette et al., Nat Comm 10:4968 (2019). Briefly, efficient protein intein splicing requires a catalytic cysteine, serine, or threonine residue in the +1 position of the C-terminal extein (the first residue of the flanking C-terminal residues) (FIG. 2). Analysis of the sequence and structure of PAH revealed four cysteine residues within the active site of the enzyme that were selected as intein split points: at positions 237 (Cys237, SEQ ID NO: 2,3), 265 (Cys265, SEQ ID NO: 4,5), 284 (Cys284, SEQ ID NO: 6,7), and 334 (Cys334, SEQ ID NO: 8,9) (FIG. 3).

Plasmids were constructed that encode the N-terminal (FIG. 4A) and C-terminal (FIG. 4B) fragments of PAH generated from using each of the four selected cysteine residues as a split point. In each plasmid, the split intein/PAH fragment was encoded downstream of the EF-1 alpha promoter on the DNA strand opposite the strand encoding the gene of interest (e.g., reporter genes mCherry (SEQ ID NO: 22), EGFP (SEQ ID NO: 21)) (FIG. 5B). A plasmid encoding full-length PAH was also generated (FIG. 5A). Promoters shown in FIGS. 4A-4B and FIGS. 5A-5B (e.g., CMV (SEQ ID NO: 45) and EF-1 alpha (SEQ ID NO: 44)) can be swapped out for any known promoter. Examples of promoters include, but are not limited to the following: CMV, EF-1 alpha, UBC, PGK, CAGG, SV40, bGH, and TRE. Strong or weak promoters (e.g., attenuated EF-1 alpha (SEQ ID NO: 43)) can be selected to tune the system for desired levels of expression of particular elements.

### Example 2: Assessing Integration of Constructs Encoding Split Selectable Markers

In order to determine whether cells transfected with plasmids encoding N-terminal and C-terminal PAH fragments were able to grow in the absence of tyrosine, plasmids encoding each split intein/PAH fragment and a reporter (e.g., mCherry, EGFP) were co-transfected into cells lacking endogenous PAH and evaluated for viability.

Briefly, plasmids encoding each PAH fragment and a reporter were co-transfected along with the piggyBac transposase plasmid in a 2:1 ratio via lipofection (Thermo LV-MAX kit) into a HEK293 cell line (also referred to as Viral Production Cells (VPCs)) (cell density=2.5×10⁶ cells/mL). Exemplary schematics are shown in FIG. 5B, in which constructs with a split point at Cys237, Cys265, Cys284, or Cys334 were tested. After 48 hours, cells were centrifuged at 300×g for 5 minutes, washed twice in DPBS, and resuspended at 1×10 6 cells/mL in serum-free media lacking tyrosine and supplemented with 300 uM (6R)-5,6,7,8-tetrahydrobiopterin (BH4). After initial selection, cells were passaged twice weekly for two weeks at 0.35-0.5×10 6 cells/mL. Viability and viable cell density (VCD) were measured at each passage on a Cellometer K2 (Nexcelom). Fluorescence was measured by flow cytometry using an Attune NxT (Thermo).

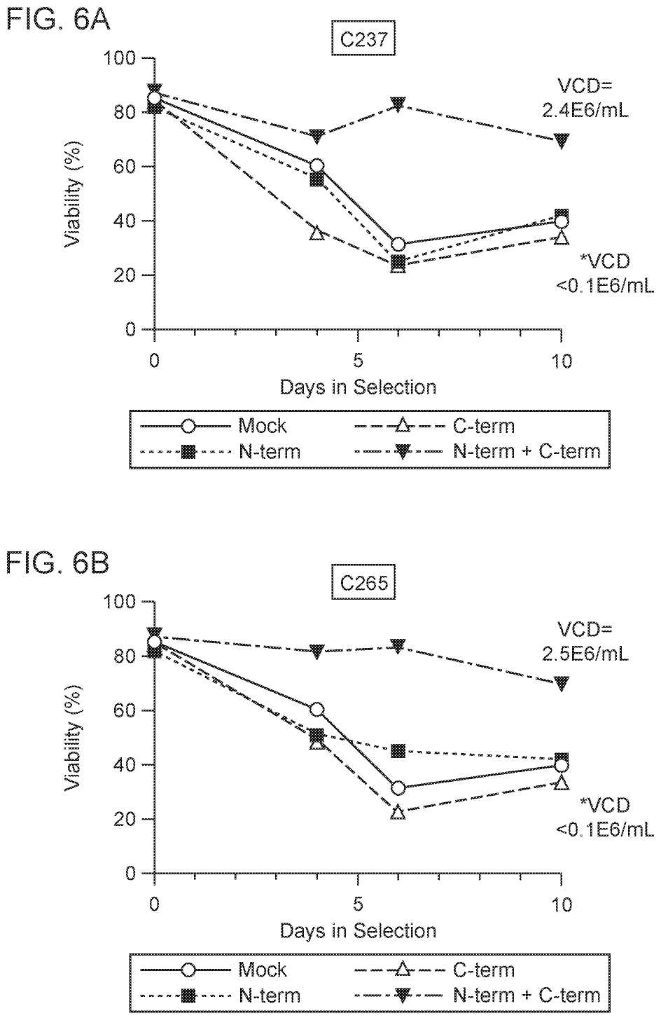

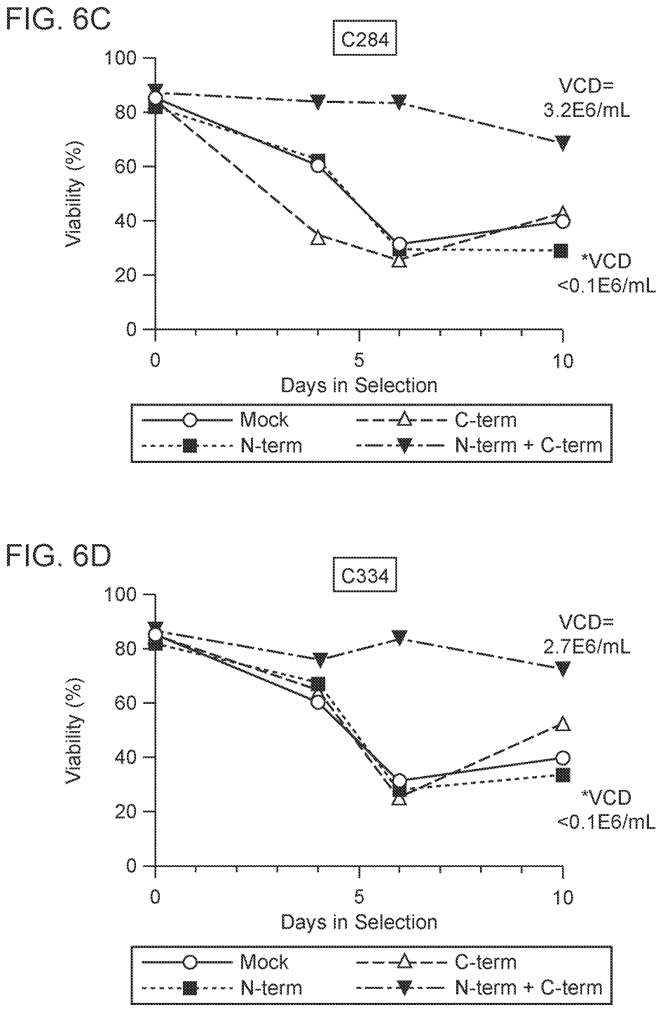

Results shown in FIGS. 6A-6D demonstrate that cells transfected with plasmids encoding both N-terminal and C-terminal PAH fragments are highly viable in media lacking tyrosine. In contrast, cells transfected with plasmids encoding only the N-terminal or C-terminal PAH fragments show significant loss of viability following selection in media lacking tyrosine, similar to the loss of viability in cells that do not express any PAH fragments (mock). The results demonstrate that each of the four selected split points (Cys237, Cys265, Cys284, and Cys334) produce N- and C-terminal fragments that have nearly equivalent splicing efficiency (comparing FIGS. 6A, 6B, 6C, and 6D).

### Example 3: PAH Selection in the Presence of 7,8-Dihydrobiopterin (7,8-BH2) Cofactor but Absence of Tetrahydrobiopterin (BH4)

Tetrahydrobiopterin (BH4) is a necessary cofactor in the PAH-catalyzed conversion of phenylalanine to tyrosine. When used in cell culture, direct dosing of BH4 is inefficient due to poor cellular retention, resulting in slow growth and poor cell viability. In addition, reconstituted synthetic BH4 is unstable with a short half-life both at room temperature and −20° C. The instability of BH4 is problematic for biopharmaceutical development because of inconsistent media compositions and increased costs associated with the higher cofactor doses.

In order to circumvent the poor stability and poor cellular retention of BH4, the BH4 precursor molecule 7,8-dihydrobiopterin (7,8-BH2) was tested during PAH selection. Full-length PAH and split intein/PAH fragments were cloned into piggyBac transposon plasmids containing either mCherry or EGFP, as described in Example 1. PAH-containing plasmids were co-transfected along with the piggyBac transposase plasmid as described in Example 2. After 48 hours, cells were centrifuged and washed as described and resuspended at 0.5×10⁶ cells/mL in serum-free media lacking tyrosine and supplemented with 200 μM 7,8-BH2. Cells were passaged in this media and assessed for viability, viable cell density, and fluorescence as described above.

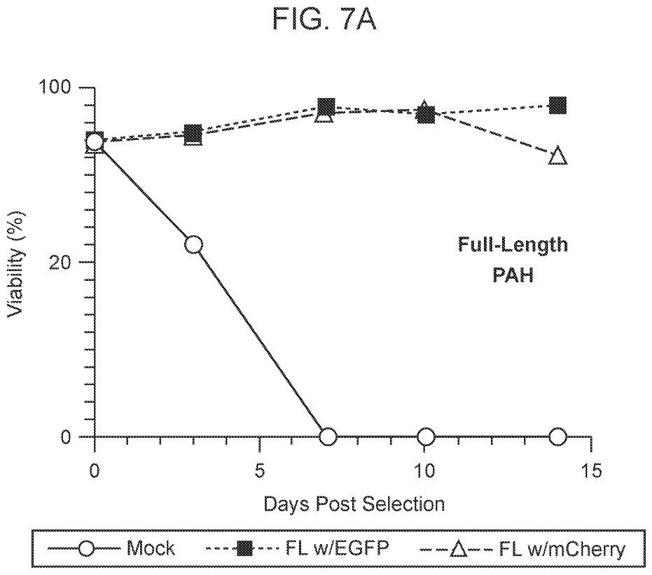

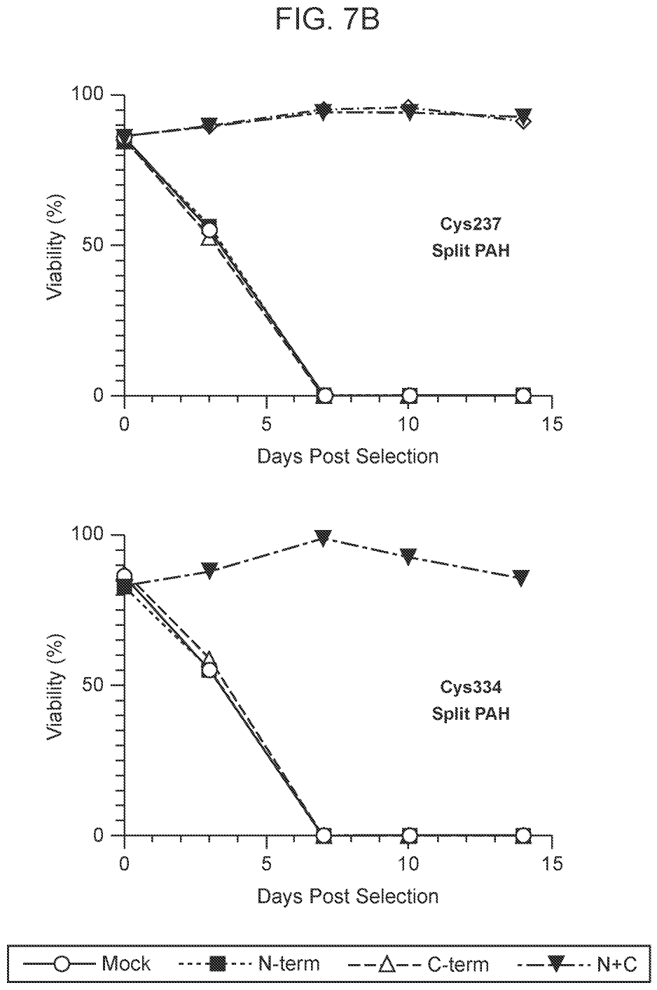

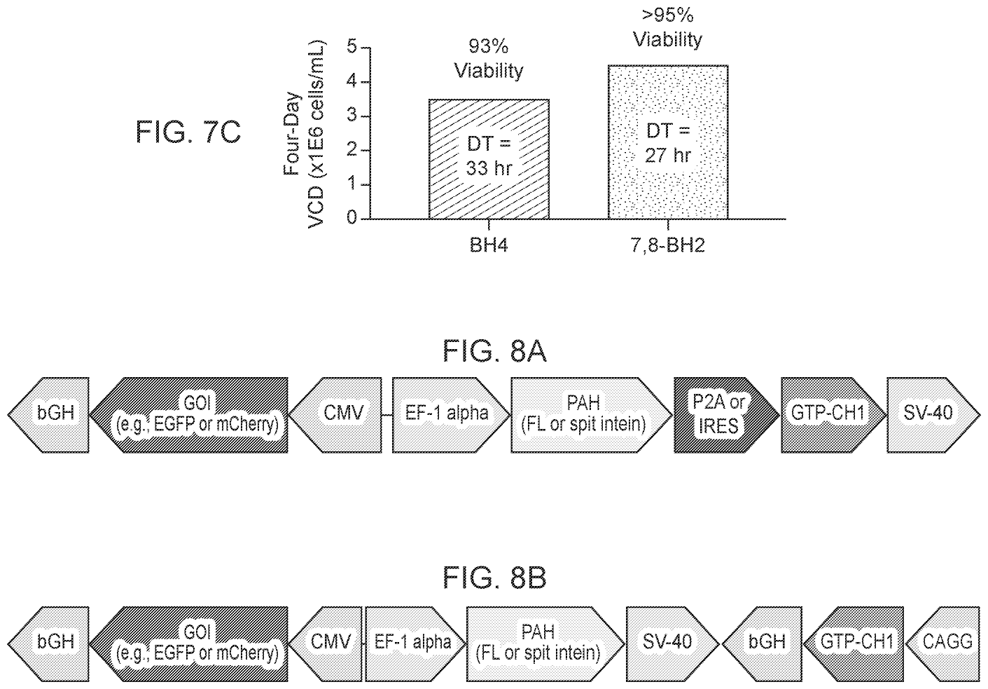

Results in FIGS. 7A and 7B show that after fourteen days of passaging in selection media containing 7,8-BH2, cells transfected with full-length PAH and both the N-terminal and C-terminal split intein/PAH fragments were at high viability and high viable cell density. Cells transfected with only the N-terminal or C-terminal split intein/PAH fragments did not survive seven days in selection. These results confirm that split intein PAH selection can be performed in cells in the absence of BH4 and the presence of 7,8-BH2.

Furthermore, cells cultured in selection media comprising 7,8-BH2 had increased viability and viable cell density after four days in selection media compared to cells cultured in selection media comprising BH4, as shown in FIG. 7C.

### Example 4: Co-Expression with GTP Cyclohydrolase I (GTP-CH1) Enables PAH Selection in the Absence of Extrinsic Cofactors

Cells that stably express PAH and are grown in the absence of BH4 or 7,8-BH2 decline in viability after 1-2 passages and eventually stagnate and die. Consequently, addition of a cofactor to the culture media has until now been a prerequisite for PAH-based selection. BH4 is synthesized in cells both from sepiapterin and from GTP. In the GTP to BH4 pathway, the first step is rate-limiting and is catalyzed by GTP cyclohydrolase I (GTP-CH1, SEQ ID NO: 10).

To determine whether GTP-CH1 overexpression in tandem with PAH can facilitate tyrosine production sufficient to support cell growth in the absence of an exogenously added cofactor, the gene encoding GTP-CH1 was cloned into a PAH-containing plasmid. Briefly, GTP-CH1 was inserted at the 3′ end of PAH (either full-length or split-intein) and separated from GOI by an internal ribosome entry site (IRES) or a P2A (SEQ ID NO: 11) self-cleaving peptide (FIG. 8A), to form PAH-P2A-(GTP-CH1) (SEQ ID NOs: 12-20) Additionally, GTP-CH1 was also inserted into a separate expression cassette with its own promoter and terminator (FIG. 8B). The resulting plasmids were integrated via piggyBac into Viral Production Cells as described in Example 2. After 48 hours, cells were centrifuged at 300×g for 5 minutes, washed twice in DPBS, and resuspended at 0.5×10⁶ cells/mL in serum-free media lacking tyrosine and absent of any cofactors. After initial selection, cells were passaged in the same media twice weekly for two weeks at 0.35-0.5×10⁶ cells/mL. Cell viability and viable cell density were measured at each passage on a Vi-Cell XR (Beckman). Fluorescence was monitored by flow cytometry using an Attune NxT (Thermo) instrument.

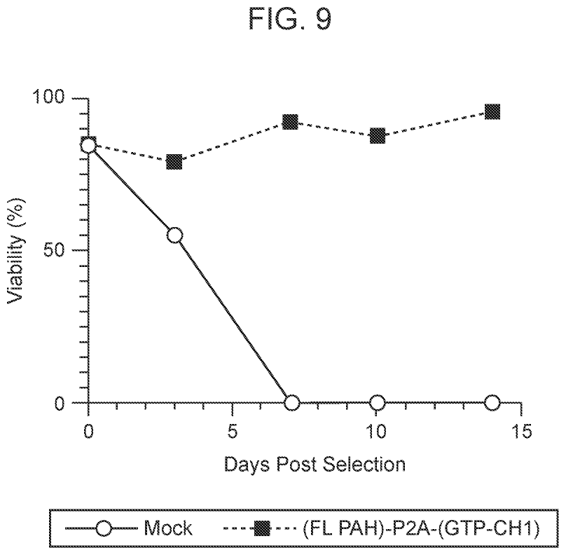

Results in FIG. 9 show that after passaging fourteen days in selection media containing no cofactors, PAH-selected cells were at high viability and high viable cell density. These results confirm that PAH selection can be performed in cells co-expressing PAH (full-length and split inteins) and GTP-CH1 in the absence of exogenous cofactors.

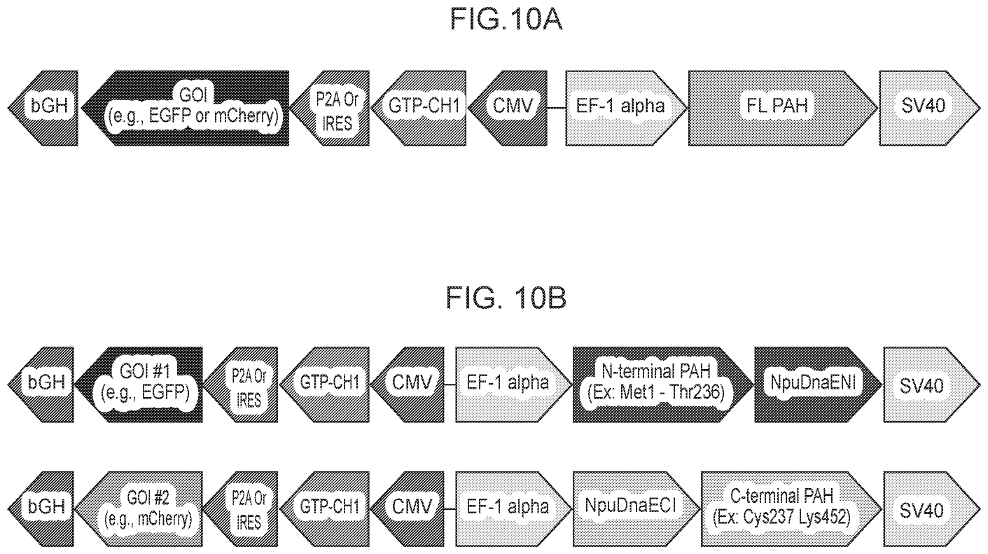

To see whether GTP-CH1 could be co-expressed adjacent to a GOI GTP-CH1 was inserted at the 5′ end of GOI and separated from GOI by an internal ribosome entry site (IRES) (FIG. 10) on both the N-terminal PAH fragment/N-terminal intein plasmid and the C-terminal intein/C-terminal PAH fragment. The resulting plasmids were integrated via piggyBac into Viral Production Cells as described in Example 2. After 48 hours, cells were centrifuged at 300×g for 5 minutes, washed twice in DPBS, and resuspended at 0.5×10 6 cells/mL in serum-free media lacking tyrosine and absent of any cofactors. After initial selection, cells were passaged in the same media twice weekly for two weeks at 0.35-0.5×10 6 cells/mL. Cell viability and viable cell density were measured at each passage on a Vi-Cell XR (Beckman). Fluorescence was monitored by flow cytometry using an Attune NxT (Thermo) instrument.

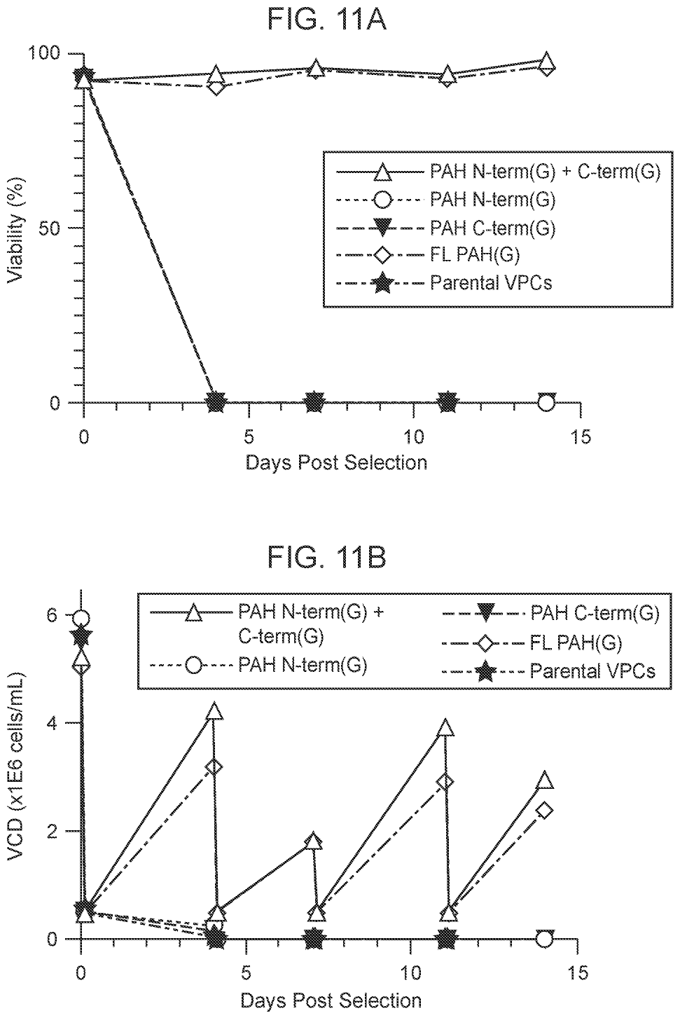

Results in FIGS. 11A-11B show that after passaging fourteen days in selection media containing no cofactors, PAH-selected cells were at high viability (FIG. 11A) and high viable cell density (FIG. 11B). These results confirm that overexpression of GTP-CH1 adjacent to the GOI on both N- and C-terminal PAH plasmids can support cell growth.

To see whether cell growth could be supported by over-expression of GTP-CH1 on only one intein-containing plasmid, plasmids combinations either + or − GTP-CH1-IRES-GOI were integrated via piggyBac into Thermo Viral Production Cells as described in Example 2. After 48 hours, cells were centrifuged at 300×g for 5 minutes, washed twice in DPBS, and resuspended at 0.5×10 6 cells/mL in serum-free media lacking tyrosine and absent of any cofactors. After initial selection, cells were passaged in the same media twice weekly for two weeks at 0.35-0.5×10 6 cells/mL. Cell viability and viable cell density were measured at each passage on a Vi-Cell XR (Beckman). Fluorescence was monitored by flow cytometry using an Attune NxT (Thermo) instrument.

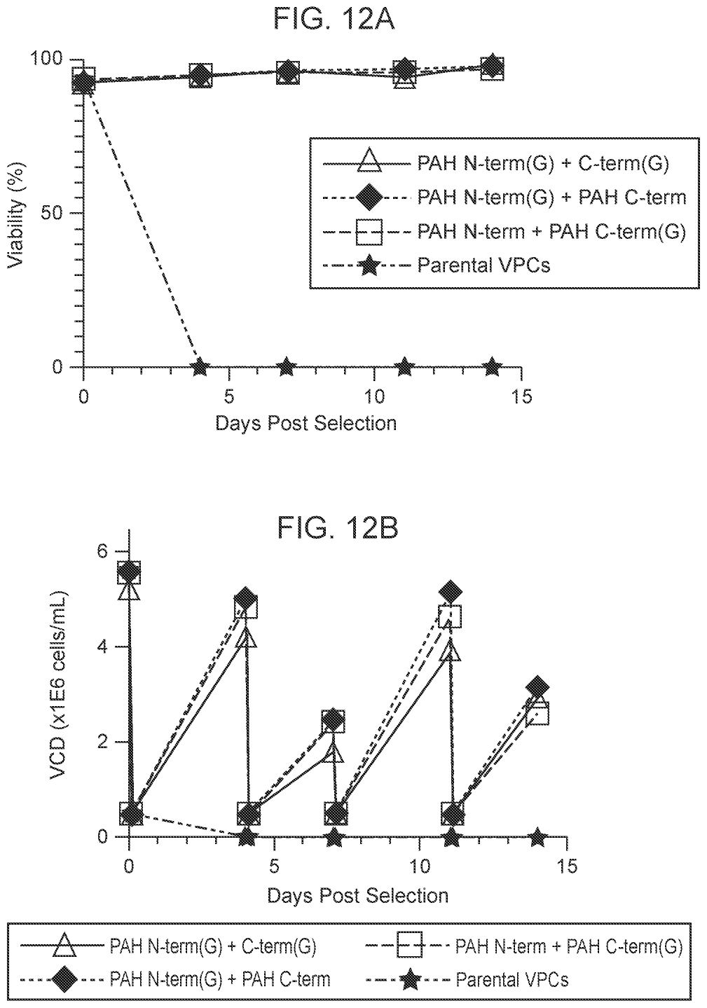

Results in FIG. 12 show that after passaging fourteen days in selection media containing no cofactors, PAH-selected cells were at high viability (FIG. 12A) and high viable cell density (FIG. 12B). These results confirm that overexpression of GTP-CH1 on only one split-intein plasmid can support cell growth.

### Example 5: Split-Intein Selection with Glutamine Synthetase

This example describes the development of a split-intein selection system using glutamine synthetase (GS, SEQ ID NO: 23). GS is an endogenous enzyme in HEK293 cells. GS catalyzes the condensation of glutamate and ammonia to glutamine HEK293 cells lacking the GS enzyme cannot grow in the absence of glutamine, as glutamine is an essential metabolite incorporated in multiple cellular processes. GS is amenable to the split-intein selection systems disclosed herein by first knocking out the enzyme in the HEK293 genome.

GS knockouts were generated in suspension HEK293 cells (Viral Production Cells (VPCs)) by genetic editing. GS knockout in these cells were confirmed by PCR and were grown in media deficient in the corresponding metabolite (GS: +/−4 mM glutamine).

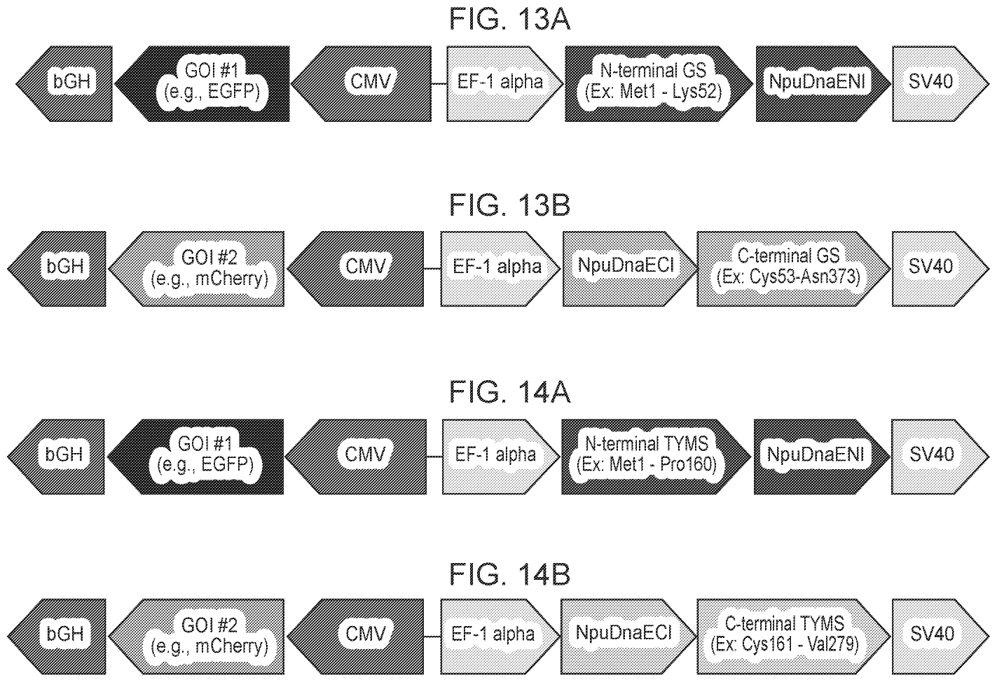

Split-intein GS constructs were designed, similar to the PAH systems disclosed herein. Non-terminal cysteine residues in GS were identified to create various split points. Each N-terminal half-enzyme was then linked to the 5′ end of the N-terminal NpuDnaE intein fragment, and each C-terminal half-enzyme was linked to the 3′ end of the C-terminal NpuDnaE intein. Possible split points for GS include: Cys 53 (FIG. 13A-FIG. 13B, SEQ ID NOS: 24-25), Cys117 (SEQ ID NOS: 26-27), Cys183 (SEQ ID NOS: 28-29), Cys229 (SEQ ID NOS: 30-31), and Cys252 (SEQ ID NOS: 32-33).

Plasmids are then integrated via piggyBac into GS Knockout VPCs in the manner described above for the PAH system. After 48 hours, cells are centrifuged at 300×g for 10 mM, washed twice in DPBS, and resuspended at 0.5×10⁶ cells/mL in serum-free media lacking glutamine After initial selection, cells are passaged in this media twice weekly for two weeks at 0.35-0.5×10⁶ cells/mL, and viability and viable cell density are measured at each passage on a Vi-CELL XR (Beckman).

### Example 6: Split-Intein Selection with Thymidylate Synthase

This example describes the development of a split-intein selection system with thymidylate synthetase (TYMS). TYMS is an endogenous enzyme in HEK293 cells. TYMS converts deoxyuridine monophosphate (dUMP) to deoxythymidine monosphosphate (dTMP). TYMS-deficient HEK293 cells cannot grow in the absence of thymidine. TYMS is amenable to the split-intein selection systems disclosed herein by first knocking out the enzyme in the HEK293 genome.

TYMS knockouts were generated in suspension HEK293 cells (Viral Production Cells (VPCs)) by genetic editing. TYMS knockout in these cells were confirmed by PCR and were grown in media deficient in the corresponding metabolite (TYMS: +/−16 mM thymidine).

Split-intein TYMS constructs were designed, similar to the PAH systems disclosed herein. Non-terminal cysteine residues in TYMS were identified to create various split points. Each N-terminal TYMS fragment was then linked to the 5′ end of the N-terminal NpuDnaE intein fragment, and each C-terminal TYMS fragment was linked to the 3′ end of the C-terminal NpuDnaE intein. Possible split points for TYMS include: Cys41 (SEQ ID NO: 35-56), Cys161 (SEQ ID NO: 37-38) (FIG. 14A-FIG. 14B), Cys165 (SEQ ID NO: 39-40), and Cys176 (SEQ ID NO: 41-42).

Plasmids are then integrated via piggyBac into TYMS KO VPCs in the manner described for the PAH system. After 48 hours, cells are centrifuged at 300×g for 10 mM, washed twice in DPBS, and resuspended at 0.5×10⁶ cells/mL in serum-free media lacking thymidine. After initial selection, cells are passaged in this media twice weekly for two weeks at 0.35-0.5×10⁶ cells/mL, and viability and viable cell density measured at each passage on a Vi-CELL XR (Beckman).

### Example 7: AAV Virion Production with PAH and GS-Based Selection

This example describes AAV virion production in cells using the PAH and GS systems disclosed herein for cell selection. Any full length or split-intein GS system of the present disclosure is incorporated into a polynucleotide construct encoding for one or more adenoviral helper proteins (referred to as a helper construct, e.g., a polynucleotide construct comprising SEQ ID NO: 48 or SEQ ID NO: 49). VPCs knocked out for GS are transfected with the helper construct and grown in media lacking glutamine Surviving cells containing the GS construct are selected for further transfections and are grown in tyrosine deficient media. Any set of split-intein PAH constructs of the present disclosure are incorporated into a polynucleotide construct encoding for Rep and Cap proteins (rep/cap construct; e.g., a polynucleotide construct comprising SEQ ID NO: 47) and a polynucleotide construct encoding for a gene of interest (GOI; GOI construct, wherein the GOI is between two ITRs). Surviving cells containing the PAH constructs are selected, expanded, and used for production of virions. The helper, rep/cap, and GOI constructs are transiently transfected or stably integrated into viral production cells. The GOI is a fluorescent marker or a payload. The payload is a therapeutic payload. The therapeutic payload is any gene, transgene, tRNA suppressor, guide RNA, or antisense oligonucleotide.

### Example 8: AAV Virion Production with PAH and TYMS-Based Selection

This example describes AAV virion production in cells using the PAH and TYMS systems disclosed herein for cell selection. Any full length or split-intein TYMS system of the present disclosure is incorporated into a polynucleotide construct encoding for one or more adenoviral helper proteins (referred to as a helper construct, e.g., a polynucleotide construct comprising SEQ ID NO: 48 or SEQ ID NO: 49). VPCs knocked out for TYMS are transfected with the helper construct and grown in media lacking thymidine. Surviving cells containing the TYMS construct are selected for further transfections and are grown in tyrosine deficient media. Any set of split-intein PAH constructs of the present disclosure are incorporated into a polynucleotide construct encoding for Rep and Cap proteins (rep/cap construct; e.g., a polynucleotide construct comprising SEQ ID NO: 47) and a polynucleotide construct encoding for a gene of interest (GOI; GOI construct). Surviving cells containing the PAH constructs are selected, expanded, and used for production of virions. The helper, rep/cap, and GOI constructs are transiently transfected or stably integrated into viral production cells. The GOI is a fluorescent marker or a payload. The payload is a therapeutic payload. The therapeutic payload is any transgene, tRNA suppressor, guide RNA, or antisense oligonucleotide.

### Example 9: AAV Virion Production with PAH and GS-Based Selection

This example describes AAV virion production in cells using the PAH and GS systems disclosed herein for cell selection. Any split-intein GS system of the present disclosure is incorporated into a polynucleotide construct encoding for one or more adenoviral helper proteins (referred to as a helper construct, e.g., a polynucleotide construct comprising SEQ ID NO: 48 or SEQ ID NO: 49) and a polynucleotide construct encoding for Rep and Cap proteins (referred to as a rep/cap construct; e.g., a polynucleotide construct comprising SEQ ID NO: 47). VPCs knocked out for GS are transfected with the helper construct and rep/cap construct and grown in media lacking glutamine Surviving cells containing the GS construct are selected for further transfections. Any full-length PAH constructs of the present disclosure are incorporated into a polynucleotide construct encoding for a gene of interest (GOI; GOI construct). Surviving cells containing the PAH constructs are selected, expanded, and used for production of virions. The helper, rep/cap, and GOI constructs are transiently transfected or stably integrated into viral production cells that are grown in tyrosine deficient media. The GOI is a fluorescent marker or a payload. The payload is a therapeutic payload. The therapeutic payload is any transgene, tRNA suppressor, guide RNA, or antisense oligonucleotide.

### Example 10: AAV Virion Production with PAH and TYMS-Based Selection

This example describes AAV virion production in cells using the PAH and TYMS systems disclosed herein for cell selection. Any split-intein TYMS system of the present disclosure is incorporated into a polynucleotide construct encoding for one or more adenoviral helper proteins (referred to as a helper construct, e.g., a polynucleotide construct comprising SEQ ID NO: 48 or SEQ ID NO: 49) and a polynucleotide construct encoding for Rep and Cap proteins (referred to as a rep/cap construct; e.g., a polynucleotide construct comprising SEQ ID NO: 47). VPCs knocked out for TYMS are transfected with the helper construct and rep/cap construct and grown in media lacking thymidine. Surviving cells containing the TYMS construct are selected for further transfections and are grown in tyrosine deficient media. Any full-length PAH construct of the present disclosure are incorporated into a polynucleotide construct encoding for a gene of interest (GOI; GOI construct). Surviving cells containing the PAH constructs are selected, expanded, and used for production of virions. The helper, rep/cap, and GOI constructs are transiently transfected or stably integrated into viral production cells. The GOI is a fluorescent marker or a payload. The payload is a therapeutic payload. The therapeutic payload is any transgene, tRNA suppressor, guide RNA, or antisense oligonucleotide.

### Example 11: Assessing Orientation of Split Selectable Markers in Constructs

Various different constructs coding for the N-term or C-term split PAH with varying orientations compared to other construct components were generated and tested to assess the impact of these different orientations.

Twelve different constructs were generated that encode for Rep and Cap proteins (rep/cap construct; e.g., a polynucleotide construct comprising SEQ ID NO: 47) and the C-term intein/C-term of the split PAH or that encode for a gene of interest (GOI; GOI construct) and the N-term of the split PAH/N-term intein.

The N-terminal fragment, encoding the N-terminal portion of PAH, was cloned into a piggyBac transposon plasmid in frame with the N-terminal fragment of the Nostoc punctiforme (Npu) split DnaE intein (NpuDnaE). The C-terminal fragment, encoding the C-terminal portion of PAH, was cloned into a piggyBac transposon plasmid in frame with the C-terminal fragment of NpuDnaE. The intein split point was at position 237 (Cys237, SEQ ID NO: 2,3).

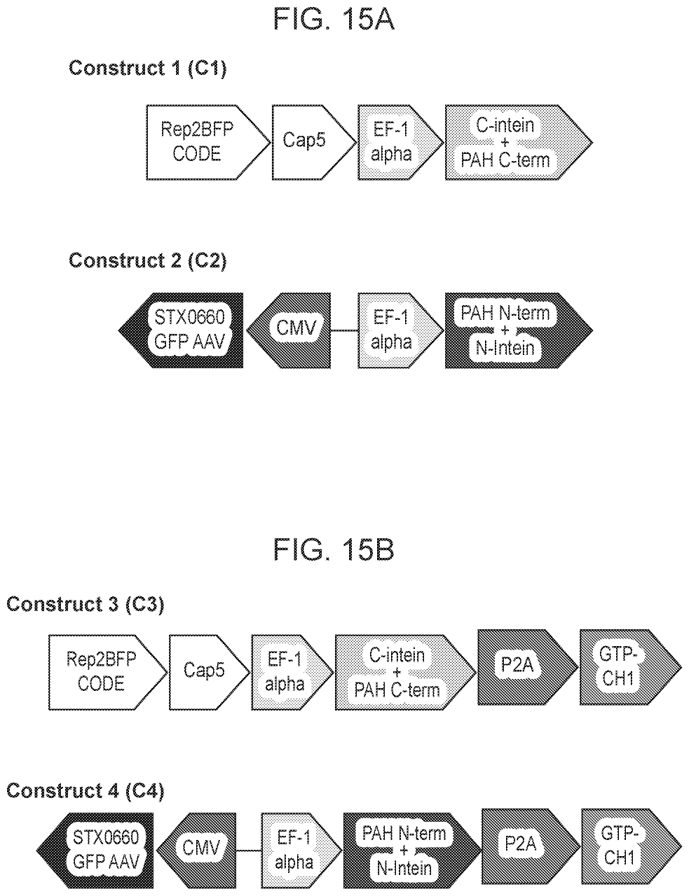

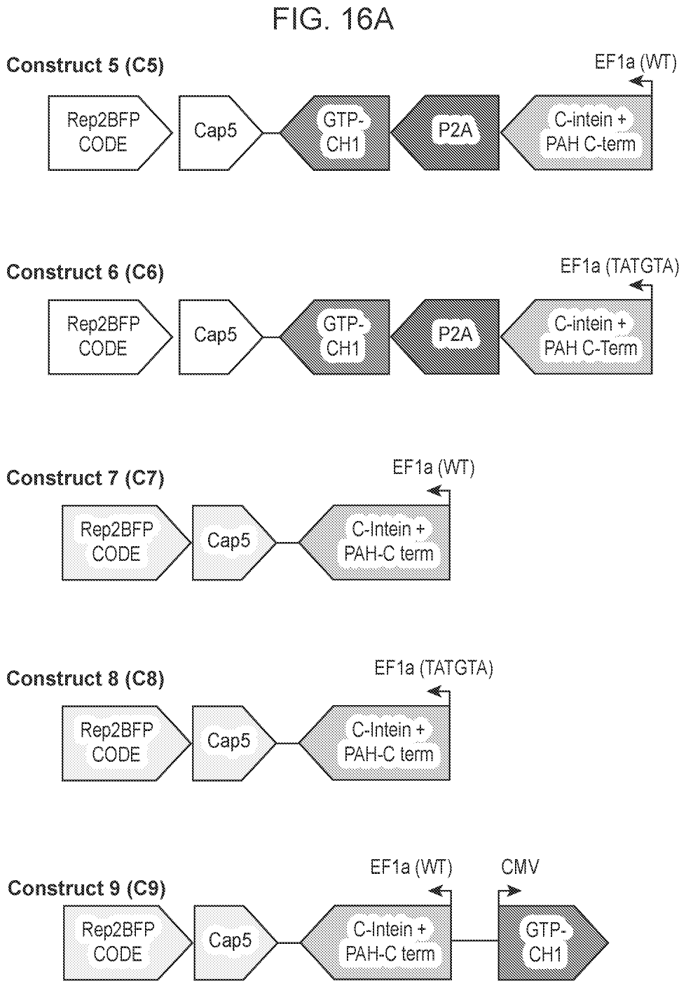

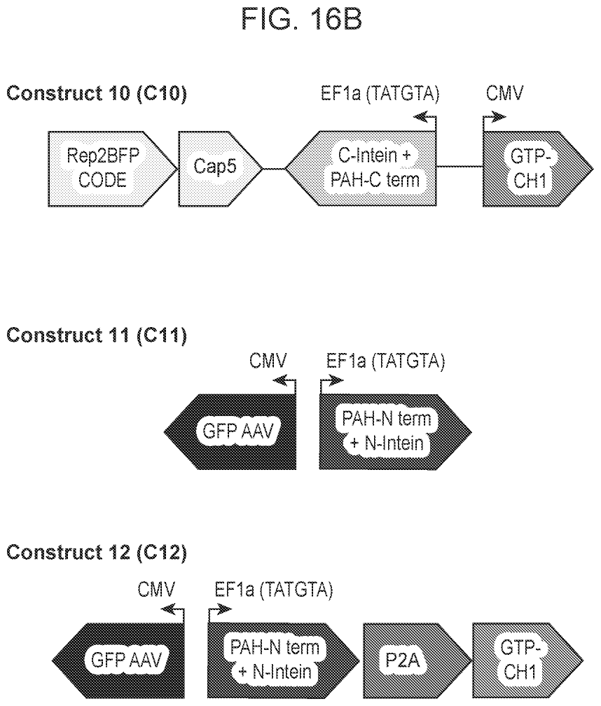

The following Rep/Cap+C Term PAH/C Term intein constructs were generated (also referred to as CODE constructs) in plasmids. Construct 1 (C1) was generated to encode for AAV Rep (Rep2BFP CODE) and Cap (Cap5) proteins (Rep2BFP CODE/Cap5: SEQ ID NO: 47) in head-to-tail orientation with EF1-alpha promoter operably linked to a C-terminal portion of an intein and a C-terminal PAH fragment. Construct 3 (C3) was generated to encode for AAV Rep (Rep2BFP CODE) and Cap (Cap5) proteins (Rep2BFP CODE/Cap5: SEQ ID NO: 47) in head-to-tail orientation with EF1-alpha promoter operably linked to a C-terminal portion of an intein and a C-terminal PAH fragment, and to P2A (a self-cleaving peptide) and GTP-CH1 (to facilitate tyrosine production and support cell growth in the absence of exogenously added cofactors). Construct 5 (C5) was generated to encode for AAV Rep (Rep2BFP CODE) and Cap (Cap5) proteins (Rep2BFP CODE/Cap5: SEQ ID NO: 47) in tail-to-tail orientation with EF1-alpha promoter operably linked to a C-terminal portion of an intein and a C-terminal PAH fragment, and to P2A and GTP-CH1. Construct 6 (C6) was generated to encode for AAV Rep (Rep2BFP CODE) and Cap (Cap5) proteins (Rep2BFP CODE/Cap5: SEQ ID NO: 47) in tail-to-tail orientation with an attenuated EF1-alpha promoter (TATGTA) operably linked to a C-terminal portion of an intein and a C-terminal PAH fragment, and to P2A and GTP-CH1. Construct 7 (C7) was generated to encode for AAV Rep (Rep2BFP CODE) and Cap (Cap5) proteins (Rep2BFP CODE/Cap5: SEQ ID NO: 47) in tail-to-tail orientation with EF1-alpha promoter operably linked to a C-terminal portion of an intein and a C-terminal PAH fragment. Construct 8 (C8) was generated to encode for AAV Rep (Rep2BFP CODE) and Cap (Cap5) proteins (Rep2BFP CODE/Cap5: SEQ ID NO: 47) in tail-to-tail orientation with an attenuated E1-alpha promoter (TATGTA) operably linked to a C-terminal portion of an intein and a C-terminal PAH fragment. Construct 9 (C9) was generated to encode for AAV Rep (Rep2BFP CODE) and Cap (Cap5) proteins (Rep2BFP CODE/Cap5: SEQ ID NO: 47) in tail-to-tail orientation with EF1-alpha promoter operably linked to a C-terminal portion of an intein and a C-terminal PAH fragment, and CMV promoter operably linked to GTP-CH1 in a head-to-head orientation with the EF1-alpha promoter operably linked to the C-terminal portion of the intein and the C-terminal PAH fragment. Construct 10 (C10) was generated to encode for AAV Rep (Rep2BFP CODE) and Cap (Cap5) proteins (Rep2BFP CODE/Cap5: SEQ ID NO: 47) in tail-to-tail orientation with an attenuated EF1-alpha promoter (TATGTA) operably linked to a C-terminal portion of an intein and a C-terminal PAH fragment, and CMV promoter operably linked to GTP-CH1 in a head-to-head orientation with the attenuated EF1-alpha promoter (TATGTA) operably linked to the C-terminal portion of the intein and the C-terminal PAH fragment. Schematics of these constructs are shown in FIGS. 15A, 15B, and 16. An EF1-alpha promoter comprises a nucleotide sequence of SEQ ID NO: 42. An attenuated ElF-alpha promoter (TATGTA) comprises nucleotide sequence of SEQ ID NO: 43.

The following Reporter+N Term intein/N Term PAH constructs (also referred to as GOI constructs) were generated in plasmids. Construct 2 (C2) was generated to encode for a CMV promoter operably linked to a gene of interest (e.g., GFP AAV) in head-to-head orientation with an E1-alpha promoter operably linked to a N-terminal PAH fragment and a N-terminal portion of an intein. Construct 4 (C4) was generated to encode for a CMV promoter operably linked to a gene of interest (e.g., GFP AAV) in head-to-head orientation with an E1-alpha promoter operably linked to a N-terminal PAH fragment and a N-terminal portion of an intein, and to P2A (a self-cleaving peptide) and GTP-CH1 (to facilitate tyrosine production and support cell growth in the absence of exogenously added cofactors). Construct 11 (C11) was generated to encode for a CMV promoter operably linked to a gene of interest (e.g., GFP AAV) in head-to-head orientation with an attenuated E1-alpha promoter (TATGTA) operably linked to a N-terminal PAH fragment and a N-terminal portion of an intein. Construct 12 (C12) was generated to encode for a CMV promoter operably linked to a gene of interest (e.g., GFP AAV) in head-to-head orientation with an attenuated E1-alpha promoter (TATGTA) operably linked to a N-terminal PAH fragment and a N-terminal portion of an intein, and to P2A (a self-cleaving peptide) and GTP-CH1 (to facilitate tyrosine production and support cell growth in the absence of exogenously added cofactors). Schematics of these constructs are shown in FIGS. 15A, 15B, and 16.

Briefly, plasmids encoding CODE construct and a GOI construct were co-transfected along with the piggyBac transposase plasmid in a 2:1 ratio via lipofection (Thermo LV-MAX kit) into a HEK293 cell line (also referred to as Viral Production Cells (VPCs)) (cell density=2.5×10⁶ cells/mL) in various combinations. After 48 hours, cells were centrifuged at 300×g for 5 minutes, washed twice in DPBS, and resuspended at 1×10 6 cells/mL in in tyrosine-deficient media containing 200 uM co-factor (BH2) or in serum-free media lacking tyrosine. Viability and viable cell density (VCD) were measured on a Cellometer K2 (Nexcelom).

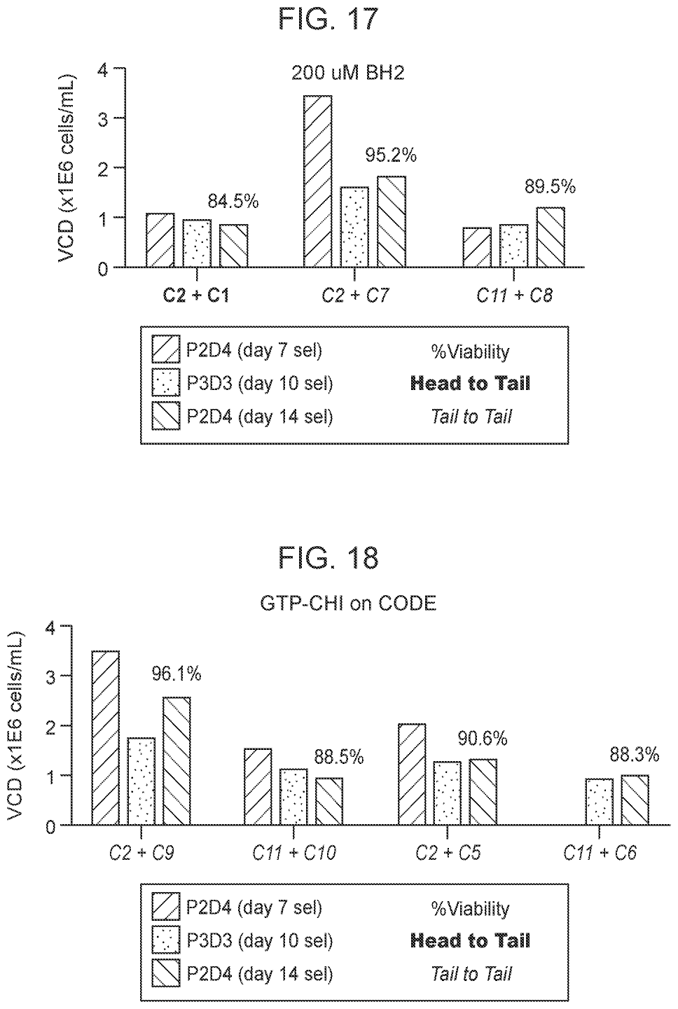

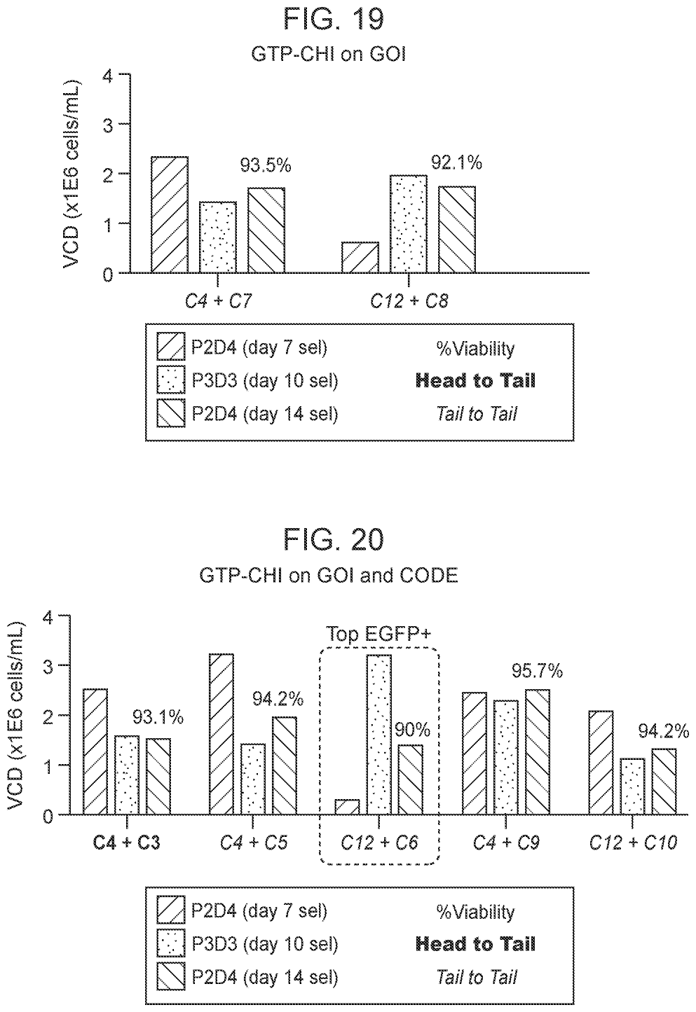

The viable cell density (VCD) of cells transfected with plasmids lacking a sequence coding for GTP-CH1 (C2 and C1; C2 and C7; or C11 and C8) following 7 days, 10 days, or 14 days selection in tyrosine-deficient media containing 200 uM co-factor (BH2) was measured (see FIG. 17). Tail-to-tail orientation in the CODE construct boosted viability. The viable cell density (VCD) of cells transfected with plasmids comprising a sequence coding for GTP-CH1 on the CODE plasmid (C2 and C9; C11 and C1; C2 and C5; or C11 and C6) following 7 days, 10 days, or 14 days selection in tyrosine-deficient media containing no cofactors was measured (see FIG. 18). The viable cell density (VCD) of cells transfected with plasmids in which the sequence encoding GTP-CH1 was only on the GOI plasmid (C4 and C7 or C12 and C8) following 7 days, 10 days, or 14 days in tyrosine-deficient media containing no cofactors (selection media). Tail-to-tail orientation in the GOI construct boosted viability was measured (see FIG. 19).

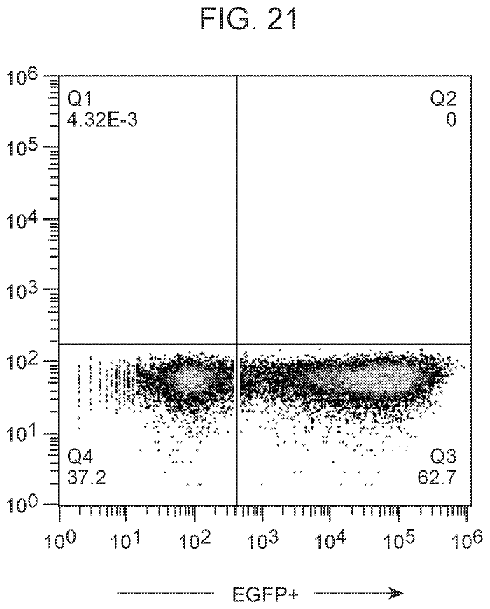

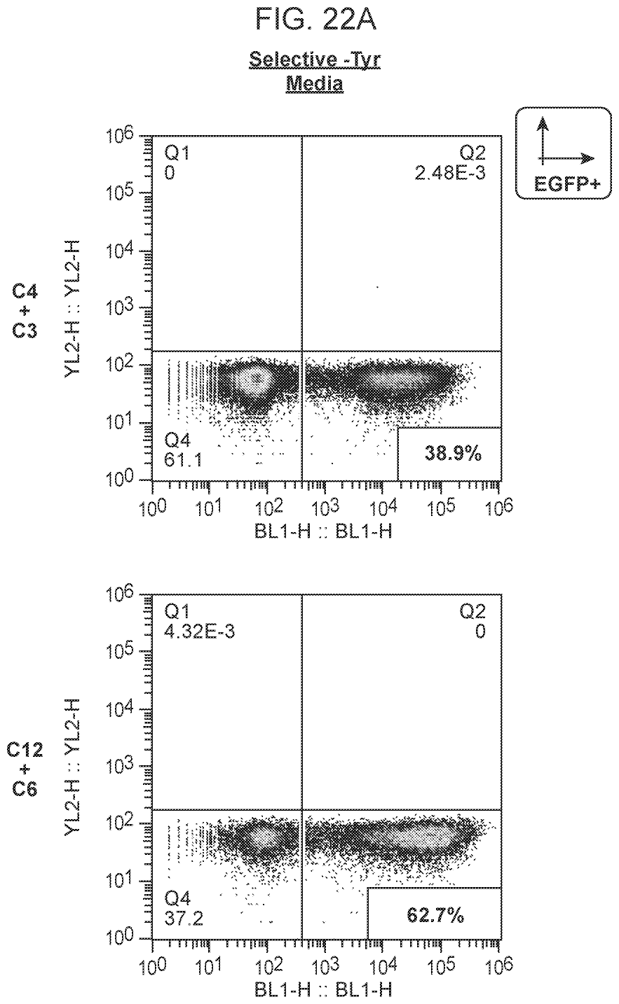

The viable cell density (VCD) of cells transfected with plasmids in which the sequence encoding the GTP-CH1 was on both the GOI plasmid and the CODE plasmid (C4 and C3; C4 and C5; C12 and C6; C4 and C9; or C12 and C10) following 7 days, 10 days, or 14 days in tyrosine-deficient media containing no cofactors (selection media) was measured (see FIG. 20). Plasmids coding for attenuated EF1apha and the P2A-GTPCHI had increased selection stringency as shown by increased EGFP+ cells. The boxed bars on the graph indicate the cells having the highest percentage of cells expressing EGFP (Top EGFP+) corresponding to the cells transfected with C12 and C6. FIG. 21 shows an exemplary flow cytometry plot for EGFP expression (x-axis) of cells from the boxed bars on the graph (cells transfected with C12 and C6) of FIG. 20.

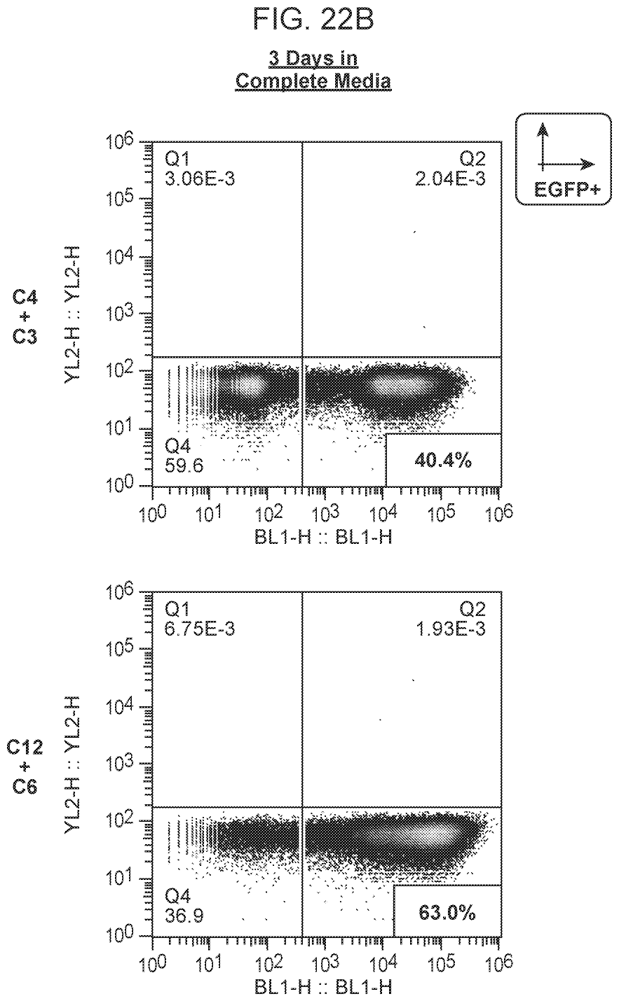

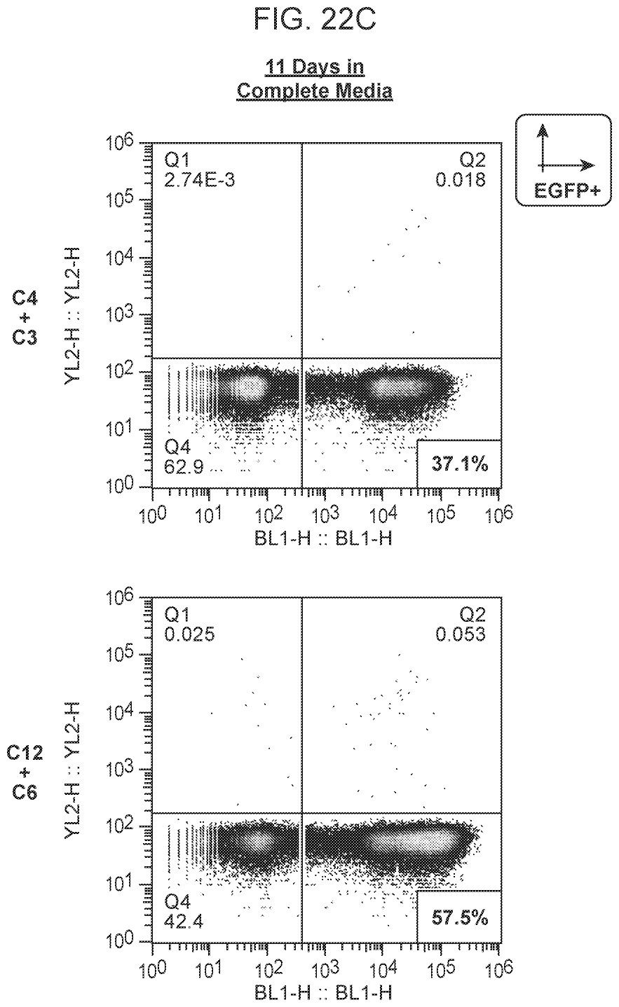

FIG. 22 shows exemplary flow cytometry plots for EGFP expression (x-axis; percentage of EGFP+ cells shown in lower right corner) for cells transfected with C4 and C3 (top plots) or C12 and C6 (bottom plots). Cells were then grown in selective media not having tyrosine (left column), for 3 days in complete media having tyrosine (middle column), or for 11 days in complete media having tyrosine (right column) Table 1 shows percent EGFP+ cells for various combinations of plasmids from FIGS. 15A, 15B, and 16. Cells transfected with both CODE plasmids and GOI plasmids coding for attenuated EF1apha promoters had an increased percentage of EGFP+ cells after culturing for two weeks in selection media compared to plasmids encoding wild-type EFlapha promoters. Cells transfected with plasmids coding for GTP-CH1 had comparable percentages of EGFP+ cells after culturing in tyrosine-deficient media containing no cofactors compared to cells transfected with plasmids not coding for GTP-CH1 and cultured in tyrosine-deficient media containing 200 uM co-factor (BH2).

TABLE 1
Percentage of cells expressing EGFP after transfection
with CODE and GOI plasmids and selection

| Plasmids | Percent EGFP+ Cells |
|---|---|
| C2 + C1 | 45.5 |
| C2 + C7 | 36.8 |
| C11 + C8 | 51.5 |
| C2 + C9 | 34.1 |
| C11 + C10 | 42.9 |
| C2 + C5 | 37.0 |
| C11 + C6 | 52.1 |
| C4 + C7 | 45.2 |
| C12 + C8 | 58 |
| C4 + C3 | 38.9 |
| C4 + C5 | 48 |
| C12 + C6 | 62.7 |
| C4 + C9 | 40.8 |
| C12 + C10 | 43.5 |

### Example 12: AAV Virion Production with Puromycin and Split-GS Selection

This example describes AAV virion production in cells using the Puromycin selection and split GS selection for selection of cells integrating plasmids encoding proteins for AAV virion production.

A plasmid encoding helper proteins and a puromycin resistance gene (helper construct, e.g., a polynucleotide construct comprising SEQ ID NO: 48 or SEQ ID NO: 49) was produced.

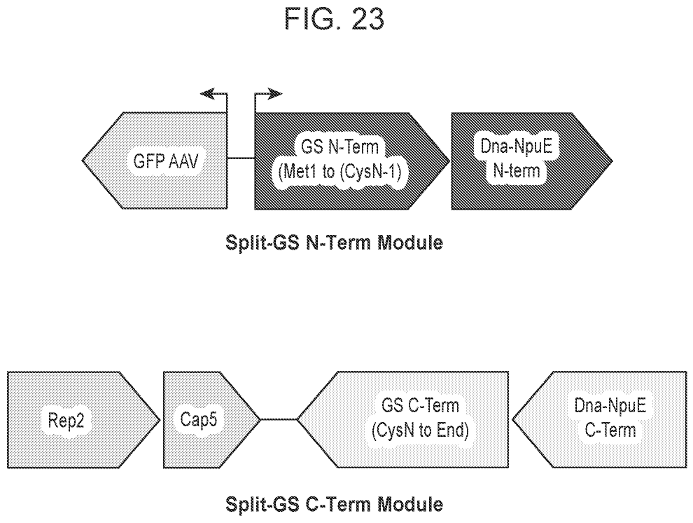

For the split GS selection, a glutamine synthetase (GS) protein was split at a Cys residue within the GS protein, in which the C-Term GS/C-Term intein was integrated into a construct encoding Rep (Rep2) and Cap (Cap5) proteins (referred to as the split-GS C-term module) and the N-Term intein/N-Term GS was integrated into a construct encoding a GFP AAV (referred to as the split-GS N-term module). Plasmids comprising the split-GS C-term module or the split-GS N-term module were generated, in which the GS split was at Cys53, Cys183, Cys229, or Cys252. FIG. 23 shows a generic schematic of a split-GS N-Term Module comprising a sequence encoding the N terminus of a split GS (which can be split at a residue directly preceding a Cys residue N (Mal to (CysN-1))) and an N terminus of a split intein (Dna-NpuE N-terminus) as well as a sequence encoding GFP AAV and a generic schematic of a split-GS C-Term Module comprising a sequence encoding a C terminus of the split intein (Dna-NpuE C-terminus) and the C terminus of the split GS (which starts at the Cys N residue of the split-GS N-Term Module (CysN to End)) and as well as a sequence encoding the Rep and Cap proteins (Rep2 and Cap5) for AAV production.

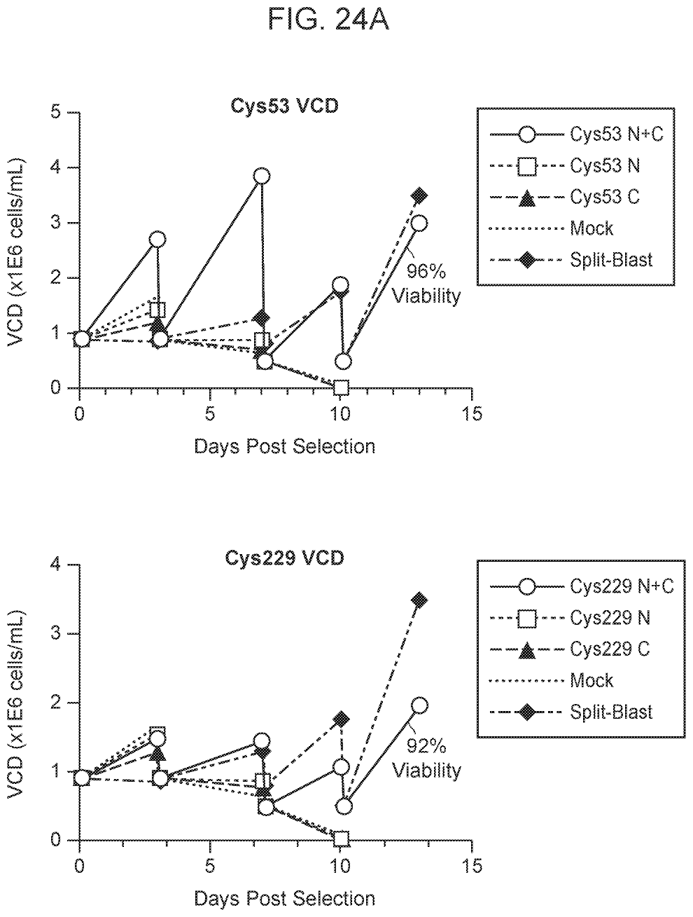

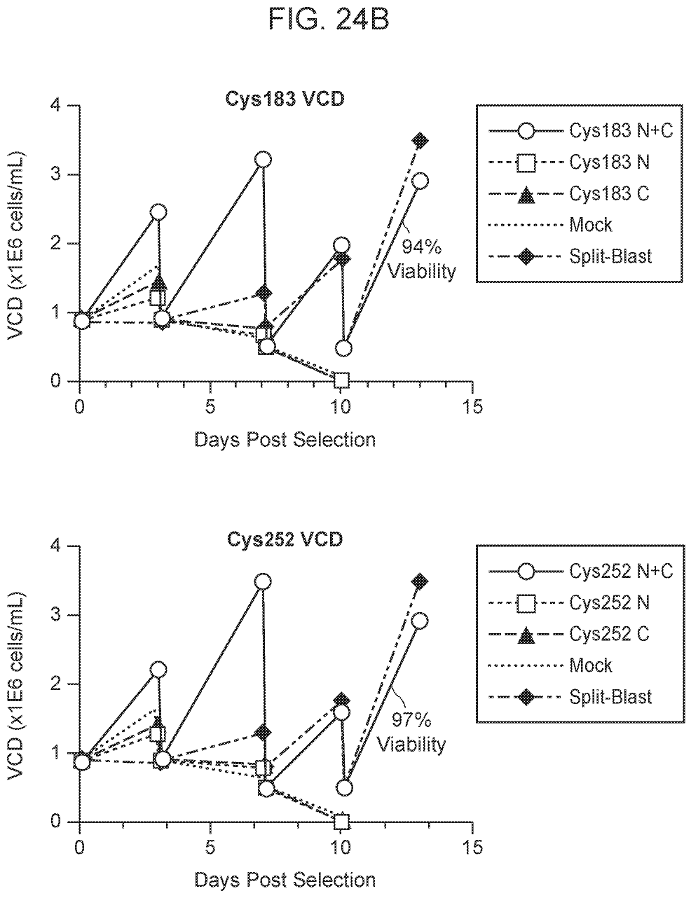

Cells for virion production were produced by transfecting a GS KO parent cell (parental viral producer cell (VPC)) as described in Example 5 with a plasmids coding for helper proteins and a puromycin resistant protein (helper construct, e.g., a polynucleotide construct comprising SEQ ID NO: 48 or SEQ ID NO: 49). These cells were cultured in media comprising puromycin to select for integration of the helper construct. Next, these cells were transfected with a plasmid comprising the split-GS N-Term Module and a plasmid comprising the split-GS C-Term Module, or plasmids encoding various controls: the split-GS N-Term Module only, the split-GS C-Term Module only, no split-GS modules (mock), or the N-term of a split Blasticidin module and the C-term of a split Blasticidin module (same as split-GS modules but the N-term and C-term of GS was replaced with a N-term and a C-term of a blasticidin resistant protein). These cells were cultured in media having no glutamine (selection media) (or selection media comprising Blasticidin for cells that were transfected with the the split Blasticidin module plasmids) and then VCD was measured at various time points out to 15 days after switching to the selection media. The viable cell density (VCD) of these transfected cells, in which the different split GS modules were tested, which is shown as indicated in FIG. 24: top left graph tested a split at Cys53; top right test a split at Cys183; bottom left tested a split at Cys229; and bottom right tested a split at Cys252.

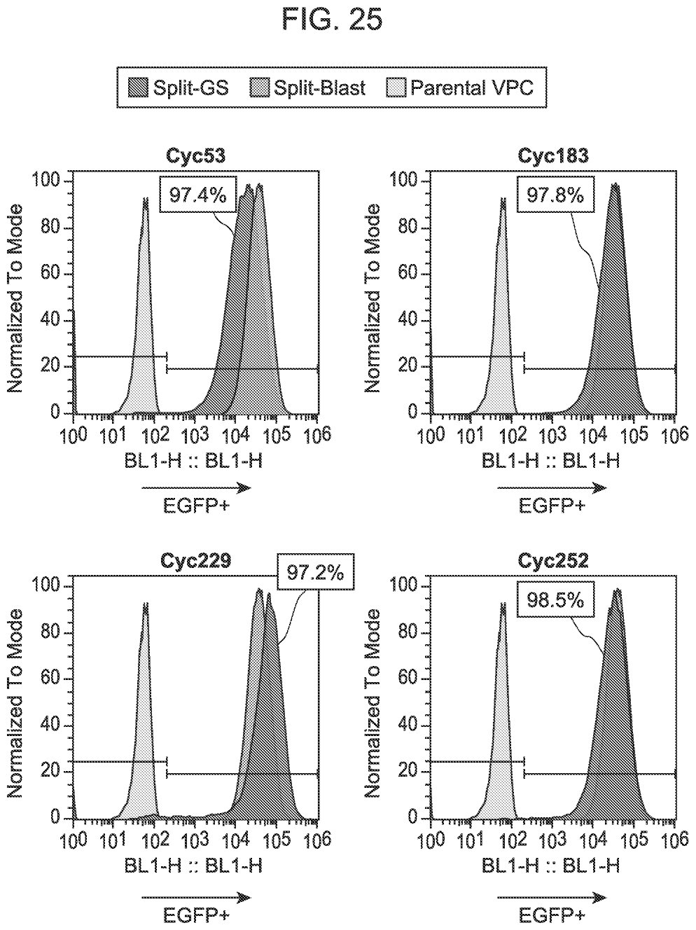

The cells were then tested for EGFP expression. The percentage of cells expressing EGFP in the cells transfected with a plasmid comprising the split-GS N-Term Module and a plasmid comprising the split-GS C-Term Module compared to cells transfected with a plasmid comprising N-term of a split Blasticidin module and a plasmid comprising the C-term of a split Blasticidin module (positive control) or a parental VPC (negative control) is shown in FIG. 25. The split GS modules tested were, from left to right, a split at Cys53, a split at Cys183, a split at Cys229, or a split at Cys252.

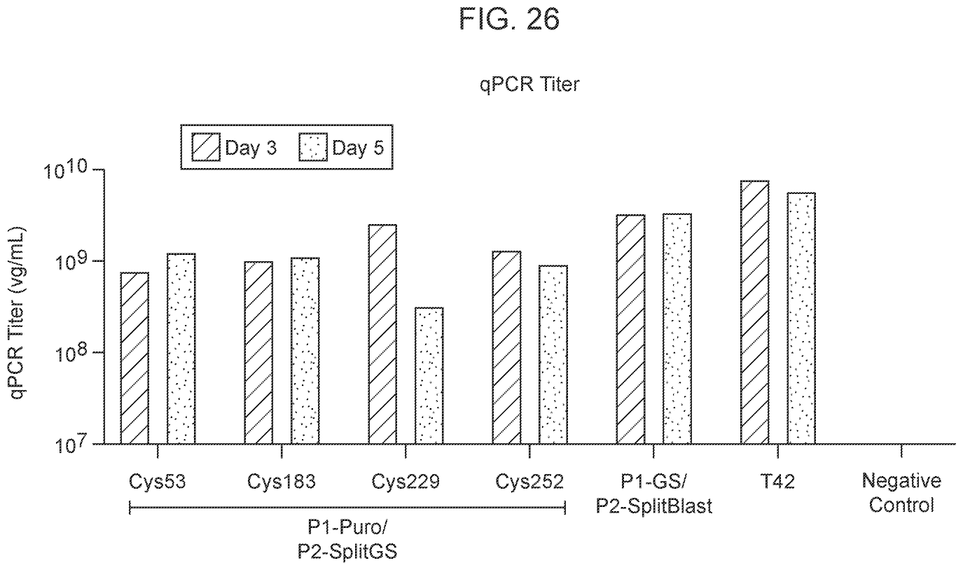

After induction of these cells, the titer of virions (vg/ml) was assessed as measured by qPCR and shown in FIG. 26. Titer of virion was assessed for cells having integrated helper constructs, the split-GS N-Term Module and the split-GS C-Term Module (P1-Puro/P2-SplitGS) in which split GS modules tested were, from left to right, a split at Cys53, a split at Cys183, a split at Cys229, or a split at Cys252; cells transfected with a helper construct coding for a GS protein instead of puromycin resistance gene followed by transfection with constructs coding for the N-term of a split Blasticidin module and the C-term of a split Blasticidin module instead of the split-GS N-Term Module and the split-GS C-Term Module (P1-GS/P2-SplitBlast); cells transfected with a helper construct coding for a puromycin resistance gene followed by transfection with constructs coding for the N-term of a split Blasticidin module and the C-term of a split Blasticidin module instead of the split-GS N-Term Module and the split-GS C-Term Module (T42); or a negative control. Titer was measured at either day 3 post-induction of virion (left bar) or day 5 post-induction of virion (right bar) for each type of transfected cell.

### Example 13: Selection of Cells Comprising a High Construct Copy Number Using an Attenuated Promoter

This example describes selection of cells comprising a high copy number of a construct (e.g., a construct comprising a sequence of interest and a selectable marker) integrated into a cell using an attenuated promoter.

A plasmid encoding helper proteins and a puromycin resistance gene (helper construct, e.g., a polynucleotide construct comprising SEQ ID NO: 48 or SEQ ID NO: 49) is produced.

A glutamine synthetase (GS) protein is split at a Cys residue within the GS protein, in which an attenuated promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid, and an attenuated promoter operably linked to an N-Term intein/N-Term GS (referred to as the split-GS N-term module) is integrated into a construct encoding a GFP AAV (e.g., SEQ ID NO: 52) to produce the N-term GS GOI plasmid. C-term GS Rep/Cap plasmids and N-term GS GOI plasmids comprising the split-GS C-term module or the split-GS N-term module are generated, in which the GS split is at Cys53, Cys183, Cys229, or Cys252. The attenuated promoter is an attenuated EF1alpha promoter having a sequence of SEQ ID NO: 43.

VPCs knocked out for GS are transfected with the helper construct and grown in media having puromycin. Surviving cells containing the helper construct are further transfected (independently for each GS split pair) with C-term GS Rep/Cap plasmids and N-term GS GOI plasmids, and then are cultured in media deficient in glutamine, expanded, and the copy number integration of C-term GS Rep/Cap constructs and N-term GS GOI constructs are assessed.

### Example 14: Selection of Cells Comprising a High Construct Copy Number Using a Selectable Marker with Weak Activity

This example describes selection of cells comprising a high copy number of a construct (e.g., a construct comprising a sequence of interest and a selectable marker) integrated into a cell using a selectable marker having weak activity, such as a selectable marker mutated to have decreased activity.

A plasmid encoding helper proteins and a puromycin resistance gene (helper construct, e.g., a polynucleotide construct comprising SEQ ID NO: 48 or SEQ ID NO: 49) is produced.

A glutamine synthetase (GS) protein is split at a Cys residue within the GS protein, in which a promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid and a promoter operably linked to an N-Term intein/N-Term GS (referred to as the split-GS N-term module) is integrated into a construct encoding a GFP AAV (e.g., SEQ ID NO: 52) to produce the N-term GS GOI plasmid. C-term GS Rep/Cap plasmids and N-term GS GOI plasmids comprising the split-GS C-term module or the split-GS N-term module are generated, in which the GS split is at Cys53, Cys183, Cys229, or Cys252, and wherein the GS is a mutated GS having a R324C, R324S, or R341C mutation as compared to SEQ ID NO: 23. The promoter is an EF1alpha promoter having a sequence of SEQ ID NO: 44.

VPCs knocked out for GS are transfected with the helper construct and grown in media having puromycin. Surviving cells containing the helper construct are further transfected (independently for each GS split pair) with C-term GS Rep/Cap plasmids and N-term GS GOI plasmids, and then are cultured in media deficient in glutamine, expanded, and the copy number integration of C-term GS Rep/Cap constructs and N-term GS GOI constructs are assessed.

### Example 15: Selection of Cells Comprising a High Construct Copy Number by Culturing Cells with an Inhibitor of a Selectable Marker

This example describes selection of cells comprising a high copy number of a construct (e.g., a construct comprising a sequence of interest and a selectable marker) integrating into a cell by culturing the cells with an inhibitor of a selectable marker.

A plasmid encoding helper proteins and a puromycin resistance gene (helper construct, e.g., a polynucleotide construct comprising SEQ ID NO: 48 or SEQ ID NO: 49) is produced.

A glutamine synthetase (GS) protein is split at a Cys residue within the GS protein, in which a promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid, and a promoter operably linked to an N-Term intein/N-Term GS (referred to as the split-GS N-term module) is integrated into a construct encoding a GFP AAV (e.g., SEQ ID NO: 52) to produce the N-term GS GOI plasmid. C-term GS Rep/Cap plasmids and N-term GS GOI plasmids comprising the split-GS C-term module or the split-GS N-term module are generated in which the GS split is at Cys53, Cys183, Cys229, or Cys252. The promoter is an EF1alpha promoter having a sequence of SEQ ID NO: 44.

VPCs knocked out for GS are transfected with the helper construct and grown in media having puromycin. Surviving cells containing the helper construct are further transfected (independently for each GS split pair) with C-term GS Rep/Cap plasmids and N-term GS GOI plasmids and then are cultured in media deficient in glutamine and comprising 0 uM, 50 uM, 125 uM, 250 uM, or 500 uM MSX, expanded, and the copy number integration of C-term GS Rep/Cap constructs and N-term GS GOI constructs are assessed.

### EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents

While the invention has been particularly shown and described with reference to a preferred embodiment and various alternate embodiments, it will be understood by persons skilled in the relevant art that various changes in form and details can be made therein without departing from the spirit and scope of the invention.

### INFORMAL SEQUENCE LISTING

The below table shows sequences of the present disclosure. Formatting (e.g., bold, bold and underlining) of an element in the description column corresponds to the element in the sequence.

| SEQ ID NO | DESCRIPTION | SEQUENCE |
|---|---|---|
| SEQ ID NO: 1 | Full-length Phenylalanine | MSTAVLENPGLGRKLSDF |
| | Hydroxylase (PAH) | GQETSYIEDNCNQNGAIS |
| | | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICHELLG |
| | | HVPLFSDRSFAQFSQEIGL |
| | | ASLGAPDEYIEKLATIYW |
| | | FTVEFGLCKQGDSIKAYG |
| | | AGLLSSFGELQYCLSEKP |
| | | KLLPLELEKTAIQNYTVT |
| | | EFQPLYYVAESFNDAKEK |
| | | VRNFAATIPRPFSVRYDP |
| | | YTQRIEVLDNTQQLKILA |
| | | DSINSEIGILCSALQKIK |
| SEQ ID NO: 2 | N-terminal PAH fragment | MSTAVLENPGLGRKLSDF |
| | C237/N-terminal NpuDnaE | GQETSYIEDNCNQNGAIS |
| | intein | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCLSYETEILTVEYGLLP |
| | | IGKIVEKRIECTVYSVDN |
| | | NGNIYTQPVAQWHDRG |
| | | EQEVFEYCLEDGSLIRA |
| | | TKDHKFMTVDGQMLPI |
| | | DEIFERELDLMRVDNLP |
| | | N |
| SEQ ID NO: 3 | C-terminal NpuDnaE | MIKIATRKYLGKQNVYD |
| | intein/C-terminal PAH | IGVERDHNFALKNGFIA |
| | fragment C237 | SNCTGFRLRPVAGLLSSR |
| | | DFLGGLAFRVFHCTQYIR |
| | | HGSKPMYTPEPDICHELL |
| | | GHVPLFSDRSFAQFSQEIG |
| | | LASLGAPDEYIEKLATIY |
| | | WFTVEFGLCKQGDSIKAY |
| | | GAGLLSSFGELQYCLSEK |
| | | PKLLPLELEKTAIQNYTVT |
| | | EFQPLYYVAESENDAKEK |
| | | VRNFAATIPRPFSVRYDP |
| | | YTQRIEVLDNTQQLKILA |
| | | DSINSEIGILCSALQKIK |
| SEQ ID NO: 4 | N-terminal PAH fragment | MSTAVLENPGLGRKLSDF |
| | C265/N-terminal NpuDnaE | GQETSYIEDNCNQNGAIS |
| | intein | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCLSYETE |
| | | ILTVEYGLLPIGKIVEKR |
| | | IECTVYSVDNNGNIYTQP |
| | | VAQWHDRGEQEVFEYC |
| | | LEDGSLIRATKDHKFMT |
| | | VDGQMLPIDEIFERELD |
| | | LMRVDNLPN |
| SEQ ID NO: 5 | C-terminal NpuDnaE | MIKIATRKYLGKQNVYD |
| | intein/C-terminal PAH | IGVERDHNFALKNGFIA |
| | fragment C265 | SNCTQYIRHGSKPMYTPE |
| | | PDICHELLGHVPLFSDRSF |
| | | AQFSQEIGLASLGAPDEYI |
| | | EKLATIYWFTVEFGLCKQ |
| | | GDSIKAYGAGLLSSFGEL |
| | | QYCLSEKPKLLPLELEKT |
| | | AIQNYTVTEFQPLYYVAE |
| | | SFNDAKEKVRNFAATIPR |
| | | PFSVRYDPYTQRIEVLDN |
| | | TQQLKILADSINSEIGIL |
| | | CSALQKIK |
| SEQ ID NO: 6 | N-terminal PAH fragment | MSTAVLENPGLGRKLSDF |
| | C284/N-terminal NpuDnaE | GQETSYIEDNCNQNGAIS |
| | intein | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICLSYET |
| | | EILTVEYGLLPIGKIVEK |
| | | RIECTVYSVDNNGNIYT |
| | | QPVAQWHDRGEQEVFE |
| | | YCLEDGSLIRATKDHKF |
| | | MTVDGQMLPIDEIFERE |
| | | LDLMRVDNLPN |
| SEQ ID NO: 7 | C-terminal NpuDnaE | MIKIATRKYLGKQNVYD |
| | intein/C-terminal PAH | IGVERDHNFALKNGFIA |
| | fragment C284 | SNCHELLGHVPLFSDRSF |
| | | AQFSQEIGLASLGAPDEYI |
| | | EKLATIYWFTVEFGLCKQ |
| | | GDSIKAYGAGLLSSFGEL |
| | | QYCLSEKPKLLPLELEKT |
| | | AIQNYTVTEFQPLYYVAE |
| | | SFNDAKEKVRNFAATIPR |
| | | PFSVRYDPYTQRIEVLDN |
| | | TQQLKILADSINSEIGILCS |
| | | ALQKIK |
| SEQ ID NO: 8 | N-terminal PAH fragment | MSTAVLENPGLGRKLSDF |
| | C334/N-terminal NpuDnaE | GQETSYIEDNCNQNGAIS |
| | intein | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICHELLG |
| | | HVPLFSDRSFAQFSQEIGL |
| | | ASLGAPDEYIEKLATIYW |
| | | FTVEFGLCLSYETEILTV |
| | | EYGLLPIGKIVEKRIECT |
| | | VYSVDNNGNIYTQPVAQ |
| | | WHDRGEQEVFEYCLED |
| | | GSLIRATKDHKFMTVDG |
| | | QMLPIDEIFERELDLMR |
| | | VDNLPN |
| SEQ ID NO: 9 | C-terminal NpuDnaE | MIKIATRKYLGKQNVYD |
| | intein/C-terminal PAH | IGVERDHNFALKNGFIA |
| | fragment C334 | SNCKQGDSIKAYGAGLLS |
| | | SFGELQYCLSEKPKLLPLE |
| | | LEKTAIQNYTVTEFQPLY |
| | | YVAESFNDAKEKVRNFA |
| | | ATIPRPFSVRYDPYTQRIE |
| | | VLDNTQQLKILADSINSEI |
| | | GILCSALQKIK |
| SEQ ID NO: 10 | GTP-CH1 | MEKGPVRAPAEKPRGAR |
| | | CSNGFPERDPPRPGPSRPA |
| | | EKPPRPEAKSAQPADGW |
| | | KGERPRSEEDNELNLPNL |
| | | AAAYSSILSSLGENPQRQ |
| | | GLLKTPWRAASAMQFFT |
| | | KGYQETISDVLNDAIFDE |
| | | DHDEMVIVKDIDMFSMC |
| | | EHHLVPFVGKVHIGYLPN |
| | | KQVLGLSKLARIVEIYSRR |
| | | LQVQERLTKQIAVAITEA |
| | | LRPAGVGVVVEATHMCM |
| | | VMRGVQKMNSKTVTST |
| | | MLGVFREDPKTREEFLTLI |
| | | RS |
| SEQ ID NO: 11 | P2A | GSGATNFSLLKQAGDVEE |
| | | NPGP |
| SEQ ID NO: 12 | Full-length PAH-P2A-(GTP- | MSTAVLENPGLGRKLSDF |
| | CH1) | GQETSYIEDNCNQNGAIS |
| | | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICHELLG |
| | | HVPLFSDRSFAQFSQEIGL |
| | | ASLGAPDEYIEKLATIYW |
| | | FTVEFGLCKQGDSIKAYG |
| | | AGLLSSFGELQYCLSEKP |
| | | KLLPLELEKTAIQNYTVT |
| | | EFQPLYYVAESENDAKEK |
| | | VRNFAATIPRPFSVRYDP |
| | | YTQRIEVLDNTQQLKILA |
| | | DSINSEIGILCSALQKIKGS |
| | | GATNESLLKQAGDVEENP |
| | | GPMEKGPVRAPAEKPR |
| | | GARCSNGFPERDPPRPG |
| | | PSRPAEKPPRPEAKSAQ |
| | | PADGWKGERPRSEEDN |
| | | ELNLPNLAAAYSSILSSL |
| | | GENPQRQGLLKTPWRA |
| | | ASAMQFFTKGYQETISD |
| | | VLNDAIFDEDHDEMVIV |
| | | KDIDMFSMCEHHLVPFV |
| | | GKVHIGYLPNKQVLGLS |
| | | KLARIVEIYSRRLQVQE |
| | | RLTKQIAVAITEALRPA |
| | | GVGVVVEATHMCMVM |
| | | RGVQKMNSKTVTSTML |
| | | GVFREDPKTREEFLTLI |
| | | RS |
| SEQ ID NO: 13 | N-terminal PAH fragment | MSTAVLENPGLGRKLSDF |
| | C237/N-terminal NpuDnaE | GQETSYIEDNCNQNGAIS |
| | intein-P2A-(GTP-CH1) | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCLSYETEILTVEYGLLP |
| | | IGKIVEKRIECTVYSVDN |
| | | NGNIYTQPVAQWHDRG |
| | | EQEVFEYCLEDGSLIRA |
| | | TKDHKFMTVDGQMLPI |
| | | DEIFERELDLMRVDNLP |
| | | NGSGATNESLLKQAGDV |
| | | EENPGPMEKGPVRAPAEK |
| | | PRGARCSNGFPERDPPRP |
| | | GPSRPAEKPPRPEAKSAQ |
| | | PADGWKGERPRSEEDNE |
| | | LNLPNLAAAYSSILSSLGE |
| | | NPQRQGLLKTPWRAASA |
| | | MQFFTKGYQETISDVLND |
| | | AIFDEDHDEMVIVKDIDM |
| | | FSMCEHHLVPFVGKVHI |
| | | GYLPNKQVLGLSKLARIV |
| | | EIYSRRLQVQERLTKQIAV |
| | | AITEALRPAGVGVVVEAT |
| | | HMCMVMRGVQKMNSKT |
| | | VTSTMLGVFREDPKTREE |
| | | FLTLIRS |
| SEQ ID NO: 14 | C-terminal NpuDnaE | MIKIATRKYLGKQNVYD |
| | intein/C-terminal PAH | IGVERDHNFALKNGFIA |
| | fragment C237-P2A-(GTP- | SNCTGFRLRPVAGLLSSR |
| | CH1) | DFLGGLAFRVFHCTQYIR |
| | | HGSKPMYTPEPDICHELL |
| | | GHVPLFSDRSFAQFSQEIG |
| | | LASLGAPDEYIEKLATIY |
| | | WFTVEFGLCKQGDSIKAY |
| | | GAGLLSSFGELQYCLSEK |
| | | PKLLPLELEKTAIQNYTVT |
| | | EFQPLYYVAESENDAKEK |
| | | VRNFAATIPRPFSVRYDP |
| | | YTQRIEVLDNTQQLKILA |
| | | DSINSEIGILCSALQKIKGS |
| | | GATNESLLKQAGDVEENP |
| | | GPMEKGPVRAPAEKPRG |
| | | ARCSNGFPERDPPRPGPS |
| | | RPAEKPPRPEAKSAQPAD |
| | | GWKGERPRSEEDNELNL |
| | | PNLAAAYSSILSSLGENPQ |
| | | RQGLLKTPWRAASAMQF |
| | | FTKGYQETISDVLNDAIF |
| | | DEDHDEMVIVKDIDMFS |
| | | MCEHHLVPFVGKVHIGY |
| | | LPNKQVLGLSKLARIVEIY |
| | | SRRLQVQERLTKQIAVAIT |
| | | EALRPAGVGVVVEATHM |
| | | CMVMRGVQKMNSKTVTS |
| | | TMLGVFREDPKTREEFLT |
| | | LIRS |
| SEQ ID NO: 15 | N-terminal PAH fragment | MSTAVLENPGLGRKLSDF |
| | C265/N-terminal NpuDnaE | GQETSYIEDNCNQNGAIS |
| | intein-P2A-(GTP-CH1) | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCLSYETE |
| | | ILTVEYGLLPIGKIVEKR |
| | | IECTVYSVDNNGNIYTQP |
| | | VAQWHDRGEQEVFEYC |
| | | LEDGSLIRATKDHKFMT |
| | | VDGQMLPIDEIFERELD |
| | | LMRVDNLPNGSGATNES |
| | | LLKQAGDVEENPGPMEK |
| | | GPVRAPAEKPRGARCSNG |
| | | FPERDPPRPGPSRPAEKP |
| | | PRPEAKSAQPADGWKGE |
| | | RPRSEEDNELNLPNLAAA |
| | | YSSILSSLGENPQRQGLLK |
| | | TPWRAASAMQFFTKGYQ |
| | | ETISDVLNDAIFDEDHDE |
| | | MVIVKDIDMFSMCEHHL |
| | | VPFVGKVHIGYLPNKQVL |
| | | GLSKLARIVEIYSRRLQVQ |
| | | ERLTKQIAVAITEALRPAG |
| | | VGVVVEATHMCMVMRG |
| | | VQKMNSKTVTSTMLGVF |
| | | REDPKTREEFLTLIRS |
| SEQ ID NO: 16 | C-terminal NpuDnaE | MIKIATRKYLGKQNVYD |
| | intein/C-terminal PAH | IGVERDHNFALKNGFIA |
| | fragment C265-P2A-(GTP- | SNCTQYIRHGSKPMYTPE |
| | CH1) | PDICHELLGHVPLFSDRSF |
| | | AQFSQEIGLASLGAPDEYI |
| | | EKLATIYWFTVEFGLCKQ |
| | | GDSIKAYGAGLLSSFGEL |
| | | QYCLSEKPKLLPLELEKT |
| | | AIQNYTVTEFQPLYYVAE |
| | | SFNDAKEKVRNFAATIPR |
| | | PFSVRYDPYTQRIEVLDN |
| | | TQQLKILADSINSEIGILCS |
| | | ALQKIKGSGATNESLLKQ |
| | | AGDVEENPGPMEKGPVR |
| | | APAEKPRGARCSNGFPER |
| | | DPPRPGPSRPAEKPPRPE |
| | | AKSAQPADGWKGERPRS |
| | | EEDNELNLPNLAAAYSSI |
| | | LSSLGENPQRQGLLKTPW |
| | | RAASAMQFFTKGYQETIS |
| | | DVLNDAIFDEDHDEMVI |
| | | VKDIDMFSMCEHHLVPF |
| | | VGKVHIGYLPNKQVLGLS |
| | | KLARIVEIYSRRLQVQERL |
| | | TKQIAVAITEALRPAGVG |
| | | VVVEATHMCMVMRGVQ |
| | | KMNSKTVTSTMLGVFRE |
| | | DPKTREEFLTLIRS |
| SEQ ID NO: 17 | N-terminal PAH fragment | MSTAVLENPGLGRKLSDF |
| | C284/N-terminal NpuDnaE | GQETSYIEDNCNQNGAIS |
| | intein-P2A-(GTP-CH1) | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICLSYET |
| | | EILTVEYGLLPIGKIVEK |
| | | RIECTVYSVDNNGNIYT |
| | | QPVAQWHDRGEQEVFE |
| | | YCLEDGSLIRATKDHKF |
| | | MTVDGQMLPIDEIFERE |
| | | LDLMRVDNLPNGSGATN |
| | | FSLLKQAGDVEENPGPME |
| | | KGPVRAPAEKPRGARCSN |
| | | GFPERDPPRPGPSRPAEK |
| | | PPRPEAKSAQPADGWKG |
| | | ERPRSEEDNELNLPNLAA |
| | | AYSSILSSLGENPQRQGLL |
| | | KTPWRAASAMQFFTKGY |
| | | QETISDVLNDAIFDEDHD |
| | | EMVIVKDIDMFSMCEHH |
| | | LVPFVGKVHIGYLPNKQV |
| | | LGLSKLARIVEIYSRRLQV |
| | | QERLTKQIAVAITEALRPA |
| | | GVGVVVEATHMCMVMR |
| | | GVQKMNSKTVTSTMLGV |
| | | FREDPKTREEFLTLIRS |
| SEQ ID NO: 18 | C-terminal NpuDnaE | MIKIATRKYLGKQNVYD |
| | intein/C-terminal PAH | IGVERDHNFALKNGFIA |
| | fragment C284-P2A-(GTP- | SNCHELLGHVPLFSDRSF |
| | CH1) | AQFSQEIGLASLGAPDEYI |
| | | EKLATIYWFTVEFGLCKQ |
| | | GDSIKAYGAGLLSSFGEL |
| | | QYCLSEKPKLLPLELEKT |
| | | AIQNYTVTEFQPLYYVAE |
| | | SFNDAKEKVRNFAATIPR |
| | | PFSVRYDPYTQRIEVLDN |
| | | TQQLKILADSINSEIGILCS |
| | | ALQKIKGSGATNESLLKQ |
| | | AGDVEENPGPMEKGPVR |
| | | APAEKPRGARCSNGFPER |
| | | DPPRPGPSRPAEKPPRPE |
| | | AKSAQPADGWKGERPRS |
| | | EEDNELNLPNLAAAYSSI |
| | | LSSLGENPQRQGLLKTPW |
| | | RAASAMQFFTKGYQETIS |
| | | DVLNDAIFDEDHDEMVI |
| | | VKDIDMFSMCEHHLVPF |
| | | VGKVHIGYLPNKQVLGLS |
| | | KLARIVEIYSRRLQVQERL |
| | | TKQIAVAITEALRPAGVG |
| | | VVVEATHMCMVMRGVQ |
| | | KMNSKTVTSTMLGVFRE |
| | | DPKTREEFLTLIRS |
| SEQ ID NO: 19 | N-terminal PAH fragment | MSTAVLENPGLGRKLSDF |
| | C334/N-terminal NpuDnaE | GQETSYIEDNCNQNGAIS |
| | intein-P2A-(GTP-CH1) | LIFSLKEEVGALAKVLRLF |
| | | EENDVNLTHIESRPSRLK |
| | | KDEYEFFTHLDKRSLPAL |
| | | TNIIKILRHDIGATVHELS |
| | | RDKKKDTVPWFPRTIQEL |
| | | DRFANQILSYGAELDADH |
| | | PGFKDPVYRARRKQFADI |
| | | AYNYRHGQPIPRVEYMEE |
| | | EKKTWGTVFKTLKSLYK |
| | | THACYEYNHIFPLLEKYC |
| | | GFHEDNIPQLEDVSQFLQ |
| | | TCTGFRLRPVAGLLSSRD |
| | | FLGGLAFRVFHCTQYIRH |
| | | GSKPMYTPEPDICHELLG |
| | | HVPLFSDRSFAQFSQEIGL |
| | | ASLGAPDEYIEKLATIYW |
| | | FTVEFGLCLSYETEILTV |
| | | EYGLLPIGKIVEKRIECT |
| | | VYSVDNNGNIYTQPVAQ |
| | | WHDRGEQEVFEYCLED |
| | | GSLIRATKDHKFMTVDG |
| | | QMLPIDEIFERELDLMR |
| | | VDNLPNGSGATNFSLLKQ |
| | | AGDVEENPGPMEKGPVR |
| | | APAEKPRGARCSNGFPER |
| | | DPPRPGPSRPAEKPPRPE |
| | | AKSAQPADGWKGERPRS |
| | | EEDNELNLPNLAAAYSSI |
| | | LSSLGENPQRQGLLKTPW |
| | | RAASAMQFFTKGYQETIS |
| | | DVLNDAIFDEDHDEMVI |
| | | VKDIDMFSMCEHHLVPF |
| | | VGKVHIGYLPNKQVLGLS |
| | | KLARIVEIYSRRLQVQERL |
| | | TKQIAVAITEALRPAGVG |
| | | VVVEATHMCMVMRGVQ |
| | | KMNSKTVTSTMLGVFRE |
| | | DPKTREEFLTLIRS |
| SEQ ID NO: 20 | C-terminal NpuDnaE | MIKIATRKYLGKQNVYD |
| | intein/C-terminal PAH | IGVERDHNFALKNGFIA |
| | fragment C334-P2A-(GTP- | SNCKQGDSIKAYGAGLLS |
| | CH1) | SFGELQYCLSEKPKLLPLE |
| | | LEKTAIQNYTVTEFQPLY |
| | | YVAESFNDAKEKVRNFA |
| | | ATIPRPFSVRYDPYTQRIE |
| | | VLDNTQQLKILADSINSEI |
| | | GILCSALQKIKGSGATNES |
| | | LLKQAGDVEENPGPMEK |
| | | GPVRAPAEKPRGARCSNG |
| | | FPERDPPRPGPSRPAEKP |
| | | PRPEAKSAQPADGWKGE |
| | | RPRSEEDNELNLPNLAAA |
| | | YSSILSSLGENPQRQGLLK |
| | | TPWRAASAMQFFTKGYQ |
| | | ETISDVLNDAIFDEDHDE |
| | | MVIVKDIDMFSMCEHHL |
| | | VPFVGKVHIGYLPNKQVL |
| | | GLSKLARIVEIYSRRLQVQ |
| | | ERLTKQIAVAITEALRPAG |
| | | VGVVVEATHMCMVMRG |
| | | VQKMNSKTVTSTMLGVF |
| | | REDPKTREEFLTLIRS |
| SEQ ID NO: 21 | EGFP | MVSKGEELFTGVVPILVE |
| | | LDGDVNGHKFSVSGEGE |
| | | GDATYGKLTLKFICTTGK |
| | | LPVPWPTLVTTLTYGVQC |
| | | FSRYPDHMKQHDFFKSA |
| | | MPEGYVQERTIFFKDDGN |
| | | YKTRAEVKFEGDTLVNRI |
| | | ELKGIDFKEDGNILGHKL |
| | | EYNYNSHNVYIMADKQK |
| | | NGIKVNFKIRHNIEDGSV |
| | | QLADHYQQNTPIGDGPVL |
| | | LPDNHYLSTQSALSKDPN |
| | | EKRDHMVLLEFVTAAGIT |
| | | LGMDELYKYSDLELK |
| SEQ ID NO: 22 | mCherry | MVSKGEEDNMAIIKEFMR |
| | | FKVHMEGSVNGHEFEIEG |
| | | EGEGRPYEGTQTAKLKVT |
| | | KGGPLPFAWDILSPQFMY |
| | | GSKAYVKHPADIPDYLKL |
| | | SFPEGFKWERVMNFEDG |
| | | GVVTVTQDSSLQDGEFIY |
| | | KVKLRGTNFPSDGPVMQ |
| | | KKTMGWEASSERMYPED |
| | | GALKGEIKQRLKLKDGG |
| | | HYDAEVKTTYKAKKPVQ |
| | | LPGAYNVNIKLDITSHNE |
| | | DYTIVEQYERAEGRHSTG |
| | | GMDELYK |
| SEQ ID NO: 23 | Full-length glutamine | MTTSASSHLNKGIKQVY |
| | synthetase (GS) | MSLPQGEKVQAMYIWID |
| | | GTGEGLRCKTRTLDSEPK |
| | | CVEELPEWNFDGSSTLQS |
| | | EGSNSDMYLVPAAMFRD |
| | | PFRKDPNKLVLCEVFKYN |
| | | RRPAETNLRHTCKRIMD |
| | | MVSNQHPWFGMEQEYTL |
| | | MGTDGHPFGWPSNGFPG |
| | | PQGPYYCGVGADRAYGR |
| | | DIVEAHYRACLYAGVKIA |
| | | GTNAEVMPAQWEFQIGP |
| | | CEGISMGDHLWVARFILH |
| | | RVCEDFGVIATFDPKPIPG |
| | | NWNGAGCHTNFSTKAMR |
| | | EENGLKYIEEAIEKLSKRH |
| | | QYHIRAYDPKGGLDNAR |
| | | RLTGFHETSNINDESAGV |
| | | ANRSASIRIPRTVGQEKK |
| | | GYFEDRRPSANCDPFSVT |
| | | EALIRTCLLNETGDEPFQY |
| | | KN |
| SEQ ID NO: 24 | N-terminal GS fragment | MTTSASSHLNKGIKQVY |
| | C53/N-terminal NpuDnaE | MSLPQGEKVQAMYIWID |
| | intein | GTGEGLRCKTRTLDSEPK |
| | | CLSYETEILTVEYGLLPI |
| | | GKIVEKRIECTVYSVDN |
| | | NGNIYTQPVAQWHDRG |
| | | EQEVFEYCLEDGSLIRA |
| | | TKDHKFMTVDGQMLPI |
| | | DEIFERELDLMRVDNLP |
| | | N |
| SEQ ID NO: 25 | C-terminal NpuDnaE | MIKIATRKYLGKQNVYD |
| | intein/C-terminal GS | IGVERDHNFALKNGFIA |
| | fragment C53 | SNCVEELPEWNEDGSSTL |
| | | QSEGSNSDMYLVPAAMF |
| | | RDPFRKDPNKLVLCEVFK |
| | | YNRRPAETNLRHTCKRIM |
| | | DMVSNQHPWFGMEQEYT |
| | | LMGTDGHPFGWPSNGFP |
| | | GPQGPYYCGVGADRAYG |
| | | RDIVEAHYRACLYAGVKI |
| | | AGTNAEVMPAQWEFQIG |
| | | PCEGISMGDHLWVARFIL |
| | | HRVCEDFGVIATFDPKPIP |
| | | GNWNGAGCHTNFSTKA |
| | | MREENGLKYIEEAIEKLS |
| | | KRHQYHIRAYDPKGGLD |
| | | NARRLTGFHETSNINDES |
| | | AGVANRSASIRIPRTVGQ |
| | | EKKGYFEDRRPSANCDPF |
| | | SVTEALIRTCLLNETGDEP |
| | | FQYKN |
| SEQ ID NO: 26 | N-terminal GS fragment | MTTSASSHLNKGIKQVY |
| | C117/N-terminal NpuDnaE | MSLPQGEKVQAMYIWID |
| | intein | GTGEGLRCKTRTLDSEPK |
| | | CVEELPEWNEDGSSTLQS |
| | | EGSNSDMYLVPAAMFRD |
| | | PFRKDPNKLVLCEVFKYN |
| | | RRPAETNLRHTCLSYETE |
| | | ILTVEYGLLPIGKIVEKR |
| | | IECTVYSVDNNGNIYTQP |
| | | VAQWHDRGEQEVFEYC |
| | | LEDGSLIRATKDHKFMT |
| | | VDGQMLPIDEIFERELD |
| | | LMRVDNLPN |
| SEQ ID NO: 27 | C-terminal NpuDnaE | MIKIATRKYLGKQNVYD |
| | intein/C-terminal GS | IGVERDHNFALKNGFIA |
| | fragment C117 | SNCKRIMDMVSNQHPWF |
| | | GMEQEYTLMGTDGHPFG |
| | | WPSNGFPGPQGPYYCGV |
| | | GADRAYGRDIVEAHYRA |
| | | CLYAGVKIAGTNAEVMP |
| | | AQWEFQIGPCEGISMGDH |
| | | LWVARFILHRVCEDFGVI |
| | | ATFDPKPIPGNWNGAGCH |
| | | TNFSTKAMREENGLKYIE |
| | | EAIEKLSKRHQYHIRAYD |
| | | PKGGLDNARRLTGFHETS |
| | | NINDESAGVANRSASIRIP |
| | | RTVGQEKKGYFEDRRPSA |
| | | NCDPESVTEALIRTCLLNE |
| | | TGDEPFQYKN |
| SEQ ID NO: 28 | N-terminal GS fragment | MTTSASSHLNKGIKQVY |
| | C183/N-terminal NpuDnaE | MSLPQGEKVQAMYIWID |
| | intein | GTGEGLRCKTRTLDSEPK |
| | | CVEELPEWNFDGSSTLQS |
| | | EGSNSDMYLVPAAMFRD |
| | | PFRKDPNKLVLCEVFKYN |
| | | RRPAETNLRHTCKRIMD |
| | | MVSNQHPWFGMEQEYTL |
| | | MGTDGHPFGWPSNGFPG |
| | | PQGPYYCGVGADRAYGR |
| | | DIVEAHYRACLSYETEIL |
| | | TVEYGLLPIGKIVEKRIE |
| | | CTVYSVDNNGNIYTQPV |
| | | AQWHDRGEQEVFEYCL |
| | | EDGSLIRATKDHKFMTV |
| | | DGQMLPIDEIFERELDL |
| | | MRVDNLPN |
| SEQ ID NO: 29 | C-terminal NpuDnaE | MIKIATRKYLGKQNVYD |
| | intein/C-terminal GS | IGVERDHNFALKNGFIA |
| | fragment C183 | SNCLYAGVKIAGTNAEV |
| | | MPAQWEFQIGPCEGISMG |
| | | DHLWVARFILHRVCEDFG |
| | | VIATFDPKPIPGNWNGAG |
| | | CHTNFSTKAMREENGLK |
| | | YIEEAIEKLSKRHQYHIRA |
| | | YDPKGGLDNARRLTGFH |
| | | ETSNINDFSAGVANRSASI |
| | | RIPRTVGQEKKGYFEDRR |
| | | PSANCDPFSVTEALIRTCL |
| | | LNETGDEPFQYKN |
| SEQ ID NO: 30 | N-terminal GS fragment | MTTSASSHLNKGIKQVY |
| | C229/N-terminal NpuDnaE | MSLPQGEKVQAMYIWID |
| | intein | GTGEGLRCKTRTLDSEPK |
| | | CVEELPEWNFDGSSTLQS |
| | | EGSNSDMYLVPAAMFRD |
| | | PFRKDPNKLVLCEVFKYN |
| | | RRPAETNLRHTCKRIMD |
| | | MVSNQHPWFGMEQEYTL |
| | | MGTDGHPFGWPSNGFPG |
| | | PQGPYYCGVGADRAYGR |
| | | DIVEAHYRACLYAGVKIA |
| | | GTNAEVMPAQWEFQIGP |
| | | CEGISMGDHLWVARFILH |
| | | RVCLSYETEILTVEYGLL |
| | | PIGKIVEKRIECTVYSVD |
| | | NNGNIYTQPVAQWHDR |
| | | GEQEVFEYCLEDGSLIR |
| | | ATKDHKFMTVDGQMLP |
| | | IDEIFERELDLMRVDNLP |
| | | N |
| SEQ ID NO: 31 | C-terminal NpuDnaE | MIKIATRKYLGKQNVYD |
| | intein/C-terminal GS | IGVERDHNFALKNGFIA |
| | fragment C229 | SNCEDFGVIATFDPKPIPG |
| | | NWNGAGCHTNFSTKAMR |
| | | EENGLKYIEEAIEKLSKRH |
| | | QYHIRAYDPKGGLDNAR |
| | | RLTGFHETSNINDFSAGV |
| | | ANRSASIRIPRTVGQEKK |
| | | GYFEDRRPSANCDPFSVT |
| | | EALIRTCLLNETGDEPFQY |
| | | KN |
| SEQ ID NO: 32 | N-terminal GS fragment | MTTSASSHLNKGIKQVY |
| | C252/N-terminal NpuDnaE | MSLPQGEKVQAMYIWID |
| | intein | GTGEGLRCKTRTLDSEPK |
| | | CVEELPEWNEDGSSTLQS |
| | | EGSNSDMYLVPAAMFRD |
| | | PFRKDPNKLVLCEVFKYN |
| | | RRPAETNLRHTCKRIMD |
| | | MVSNQHPWFGMEQEYTL |
| | | MGTDGHPFGWPSNGFPG |
| | | PQGPYYCGVGADRAYGR |
| | | DIVEAHYRACLYAGVKIA |
| | | GTNAEVMPAQWEFQIGP |
| | | CEGISMGDHLWVARFILH |
| | | RVCEDFGVIATFDPKPIPG |
| | | NWNGAGCLSYETEILTV |
| | | EYGLLPIGKIVEKRIECT |
| | | VYSVDNNGNIYTQPVAQ |
| | | WHDRGEQEVFEYCLED |
| | | GSLIRATKDHKFMTVDG |
| | | QMLPIDEIFERELDLMR |
| | | VDNLPN |
| SEQ ID NO: 33 | C-terminal NpuDnaE | MIKIATRKYLGKQNVYD |
| | intein/C-terminal GS | IGVERDHNFALKNGFIA |
| | fragment C252 | SNCHTNESTKAMREENGL |
| | | KYIEEAIEKLSKRHQYHIR |
| | | AYDPKGGLDNARRLTGF |
| | | HETSNINDFSAGVANRSA |
| | | SIRIPRTVGQEKKGYFEDR |
| | | RPSANCDPFSVTEALIRTC |
| | | LLNETGDEPFQYKN |
| SEQ ID NO: 34 | Full-length Thymidylate | MPVAGSELPRRPLPPAAQ |
| | Synthase (TYMS) | ERDAEPRPPHGELQYLGQ |
| | | IQHILRCGVRKDDRTGTG |
| | | TLSVFGMQARYSLRDEFP |
| | | LLTTKRVFWKGVLEELL |
| | | WFIKGSTNAKELSSKGVK |
| | | IWDANGSRDFLDSLGFST |
| | | REEGDLGPVYGFQWRHF |
| | | GAEYRDMESDLPLMALP |
| | | PCHALCQFYVVNSELSCQ |
| | | LYQRSGDMGLGVPFNIAS |
| | | YALLTYMIAHITGLKPGD |
| | | FIHTLGDAHIYLNHIEPLKI |
| | | QLQREPRPFPKLRILRKVE |
| | | KIDDFKAEDFQIEGYNPH |
| | | PTIKMEMAV |
| SEQ ID NO: 35 | N-terminal TYMS fragment | MPVAGSELPRRPLPPAAQ |
| | C41/N-terminal NpuDnaE | ERDAEPRPPHGELQYLGQ |
| | intein | IQHILRCLSYETEILTVEY |
| | | GLLPIGKIVEKRIECTVY |
| | | SVDNNGNIYTQPVAQW |
| | | HDRGEQEVFEYCLEDGS |
| | | LIRATKDHKFMTVDGQ |
| | | MLPIDEIFERELDLMRV |
| | | DNLPN |
| SEQ ID NO: 36 | C-terminal NpuDnaE | MIKIATRKYLGKQNVYD |
| | intein/C-terminal TYMS | IGVERDHNFALKNGFIA |
| | fragment C41 | SNCGVRKDDRTGTGTLS |
| | | VFGMQARYSLRDEFPLLT |
| | | TKRVFWKGVLEELLWFIK |
| | | GSTNAKELSSKGVKIWDA |
| | | NGSRDFLDSLGESTREEG |
| | | DLGPVYGFQWRHFGAEY |
| | | RDMESDLPLMALPPCHAL |
| | | CQFYVVNSELSCQLYQRS |
| | | GDMGLGVPFNIASYALLT |
| | | YMIAHITGLKPGDFIHTLG |
| | | DAHIYLNHIEPLKIQLQRE |
| | | PRPFPKLRILRKVEKIDDF |
| | | KAEDFQIEGYNPHPTIKM |
| | | EMAV |
| SEQ ID NO: 37 | N-terminal TYMS fragment | MPVAGSELPRRPLPPAAQ |
| | C161/N-terminal NpuDnaE | ERDAEPRPPHGELQYLGQ |
| | intein | IQHILRCGVRKDDRTGTG |
| | | TLSVFGMQARYSLRDEFP |
| | | LLTTKRVFWKGVLEELL |
| | | WFIKGSTNAKELSSKGVK |
| | | IWDANGSRDFLDSLGFST |
| | | REEGDLGPVYGFQWRHF |
| | | GAEYRDMESDLPLMALP |
| | | PCLSYETEILTVEYGLLP |
| | | IGKIVEKRIECTVYSVDN |
| | | NGNIYTQPVAQWHDRG |
| | | EQEVFEYCLEDGSLIRA |
| | | TKDHKFMTVDGQMLPI |
| | | DEIFERELDLMRVDNLP |
| | | N |
| SEQ ID NO: 38 | C-terminal NpuDnaE | MIKIATRKYLGKQNVYD |
| | intein/C-terminal TYMS | IGVERDHNFALKNGFIA |
| | fragment C161 | SNCHALCQFYVVNSELSC |
| | | QLYQRSGDMGLGVPFNIA |
| | | SYALLTYMIAHITGLKPG |
| | | DFIHTLGDAHIYLNHIEPL |
| | | KIQLQREPRPFPKLRILRK |
| | | VEKIDDFKAEDFQIEGYN |
| | | PHPTIKMEMAV |
| SEQ ID NO: 39 | N-terminal TYMS fragment | MPVAGSELPRRPLPPAAQ |
| | C165/N-terminal NpuDnaE | ERDAEPRPPHGELQYLGQ |
| | intein | IQHILRCGVRKDDRTGTG |
| | | TLSVFGMQARYSLRDEFP |
| | | LLTTKRVFWKGVLEELL |
| | | WFIKGSTNAKELSSKGVK |
| | | IWDANGSRDFLDSLGFST |
| | | REEGDLGPVYGFQWRHF |
| | | GAEYRDMESDLPLMALP |
| | | PCHALCLSYETEILTVEY |
| | | GLLPIGKIVEKRIECTVY |
| | | SVDNNGNIYTQPVAQW |
| | | HDRGEQEVFEYCLEDGS |
| | | LIRATKDHKFMTVDGQ |
| | | MLPIDEIFERELDLMRV |
| | | DNLPN |
| SEQ ID NO: 40 | C-terminal NpuDnaE | MIKIATRKYLGKQNVYD |
| | intein/C-terminal TYMS | IGVERDHNFALKNGFIA |
| | fragment C165 | SNCQFYVVNSELSCQLYQ |
| | | RSGDMGLGVPFNIASYAL |
| | | LTYMIAHITGLKPGDFIHT |
| | | LGDAHIYLNHIEPLKIQLQ |
| | | REPRPFPKLRILRKVEKID |
| | | DFKAEDFQIEGYNPHPTIK |
| | | MEMAV |
| SEQ ID NO: 41 | N-terminal TYMS fragment | MPVAGSELPRRPLPPAAQ |
| | C176/N-terminal NpuDnaE | ERDAEPRPPHGELQYLGQ |
| | intein | IQHILRCGVRKDDRTGTG |
| | | TLSVFGMQARYSLRDEFP |
| | | LLTTKRVFWKGVLEELL |
| | | WFIKGSTNAKELSSKGVK |
| | | IWDANGSRDFLDSLGFST |
| | | REEGDLGPVYGFQWRHF |
| | | GAEYRDMESDLPLMALP |
| | | PCHALCQFYVVNSELSCL |
| | | SYETEILTVEYGLLPIGK |
| | | IVEKRIECTVYSVDNNG |
| | | NIYTQPVAQWHDRGEQ |
| | | EVFEYCLEDGSLIRATK |
| | | DHKFMTVDGQMLPIDEI |
| | | FERELDLMRVDNLPN |
| SEQ ID NO: 42 | C-terminal NpuDnaE | MIKIATRKYLGKQNVYD |
| | intein/C-terminal TYMS | IGVERDHNFALKNGFIA |
| | fragment C176 | SNCQLYQRSGDMGLGVP |
| | | FNIASYALLTYMIAHITGL |
| | | KPGDFIHTLGDAHIYLNHI |
| | | EPLKIQLQREPRPFPKLRIL |
| | | RKVEKIDDFKAEDFQIEG |
| | | YNPHPTIKMEMAV |
| SEQ ID NO: 43 | Attenuated EF1alpha | AAGGATCTGCGATCGCT |
| | promoter | CCGGTGCCCGTCAGTGG |
| | | GCAGAGCGCACATCGCC |
| | | CACAGTCCCCGAGAAGT |
| | | TGGGGGGAGGGGTCGGC |
| | | AATTGAACGGGTGCCTA |
| | | GAGAAGGTGGCGCGGG |
| | | GTAAACTGGGAAAGTGA |
| | | TGTCGTGTACTGGCTCC |
| | | GCCTTTTTCCCGAGGGT |
| | | GGGGGAGAACCGTATGT |
| | | AAGTGCAGTAGTCGCCG |
| | | TGAACGTTCTTTTTCGCA |
| | | ACGGGTTTGCCGCCAGA |
| | | ACACAGCTGAAGCTTCG |
| | | AGGGGCTCGCATCTCTC |
| | | CTTCACGCGCCCGCCGC |
| | | CCTACCTGAGGCCGCCA |
| | | TCCACGCCGGTTGAGTC |
| | | GCGTTCTGCCGCCTCCC |
| | | GCCTGTGGTGCCTCCTG |
| | | AACTGCGTCCGCCGTCT |
| | | AGGTAAGTTTAAAGCTC |
| | | AGGTCGAGACCGGGCCT |
| | | TTGTCCGGCGCTCCCTTG |
| | | GAGCCTACCTAGACTCA |
| | | GCCGGCTCTCCACGCTTT |
| | | GCCTGACCCTGCTTGCTC |
| | | AACTCTACGTCTTTGTTT |
| | | CGTTTTCTGTTCTGCGCC |
| | | GTTACAGATCCAAGCTG |
| | | TGACCGGCGCCTAC |
| SEQ ID NO: 44 | EF1 alpha promoter | AAGGATCTGCGATCGCT |
| | | CCGGTGCCCGTCAGTGG |
| | | GCAGAGCGCACATCGCC |
| | | CACAGTCCCCGAGAAGT |
| | | TGGGGGGAGGGGTCGGC |
| | | AATTGAACGGGTGCCTA |
| | | GAGAAGGTGGCGCGGG |
| | | GTAAACTGGGAAAGTGA |
| | | TGTCGTGTACTGGCTCC |
| | | GCCTTTTTCCCGAGGGT |
| | | GGGGGAGAACCGTATAT |
| | | AAGTGCAGTAGTCGCCG |
| | | TGAACGTTCTTTTTCGCA |
| | | ACGGGTTTGCCGCCAGA |
| | | ACACAGCTGAAGCTTCG |
| | | AGGGGCTCGCATCTCTC |
| | | CTTCACGCGCCCGCCGC |
| | | CCTACCTGAGGCCGCCA |
| | | TCCACGCCGGTTGAGTC |
| | | GCGTTCTGCCGCCTCCC |
| | | GCCTGTGGTGCCTCCTG |
| | | AACTGCGTCCGCCGTCT |
| | | AGGTAAGTTTAAAGCTC |
| | | AGGTCGAGACCGGGCCT |
| | | TTGTCCGGCGCTCCCTTG |
| | | GAGCCTACCTAGACTCA |
| | | GCCGGCTCTCCACGCTTT |
| | | GCCTGACCCTGCTTGCTC |
| | | AACTCTACGTCTTTGTTT |
| | | CGTTTTCTGTTCTGCGCC |
| | | GTTACAGATCCAAGCTG |
| | | TGACCGGCGCCTAC |
| SEQ ID NO: 45 | CMV promoter | ACTAGTATTATGCCCAG |
| | | TACATGACCTTATGGGA |
| | | CTTTCCTACTTGGCAGTA |
| | | CATCTACGTATTAGTCAT |
| | | CGCTATTACCATGGTGA |
| | | TGCGGTTTTGGCAGTAC |
| | | ATCAATGGGCGTGGATA |
| | | GCGGTTTGACTCACGGG |
| | | GATTTCCAAGTCTCCAC |
| | | CCCATTGACGTCAATGG |
| | | GAGTTTGTTTTGGCACC |
| | | AAAATCAACGGGACTTT |
| | | CCAAAATGTCGTAACAA |
| | | CTCCGCCCCATTGACGC |
| | | AAATGGGCGGTAGGCGT |
| | | GTACGGTGGGAGGTTTA |
| | | TATAAGCAGAGCTCGTT |
| | | TAGTGAACCGTCAGATC |
| | | GCCTGGAGACGCCATCC |
| | | ACGCTGTTTTGACCTCCA |
| | | TAGAAGA |
| SEQ ID NO: 46 | IRES | GCCCCTCTCCCTCCCCCC |
| | | CCCCTAACGTTACTGGC |
| | | CGAAGCCGCTTGGAATA |
| | | AGGCCGGTGTGCGTTTG |
| | | TCTATATGTTATTTTCCA |
| | | CCATATTGCCGTCTTTTG |
| | | GCAATGTGAGGGCCCGG |
| | | AAACCTGGCCCTGTCTT |
| | | CTTGACGAGCATTCCTA |
| | | GGGGTCTTTCCCCTCTCG |
| | | CCAAAGGAATGCAAGGT |
| | | CTGTTGAATGTCGTGAA |
| | | GGAAGCAGTTCCTCTGG |
| | | AAGCTTCTTGAAGACAA |
| | | ACAACGTCTGTAGCGAC |
| | | CCTTTGCAGGCAGCGGA |
| | | ACCCCCCACCTGGCGAC |
| | | AGGTGCCTCTGCGGCCA |
| | | AAAGCCACGTGTATAAG |
| | | ATACACCTGCAAAGGCG |
| | | GCACAACCCCAGTGCCA |
| | | CGTTGTGAGTTGGATAG |
| | | TTGTGGAAAGAGTCAAA |
| | | TGGCTCTCCTCAAGCGT |
| | | ATTCAACAAGGGGCTGA |
| | | AGGATGCCCAGAAGGTA |
| | | CCCCATTGTATGGGATC |
| | | TGATCTGGGGCCTCGGT |
| | | GCACATGCTTTACATGT |
| | | GTTTAGTCGAGGTTAAA |
| | | AAAACGTCTAGGCCCCC |
| | | CGAACCACGGGGACGTG |
| | | GTTTTCCTTTGAAAAAC |
| | | ACGATGATAATATGGCC |
| | | ACAACC |
| SEQ ID NO: 47 | Rep2BFP-CODE/Cap5 | GGAGGGGTGGAGTCGTG |
| | construct | ACGTGAATTACGTCATA |
| | | GGGTTAGGGAGGTCCTG |
| | | TATTAGAGGTCACGTGA |
| | | GTGTTTTGCGACATTTTG |
| | | CGACACCATGTGGTCAC |
| | | GCTGGGTATTTAAGCCC |
| | | GAGTGAGCACGCAGGGT |
| | | CTCCATTTTGAAGCGGG |
| | | AGGTTTGAACGCGCAGC |
| | | CGCCATGCCGGGGTTTT |
| | | ACGAGATTGTGATTAAG |
| | | GTCCCCAGCGACCTTGA |
| | | CGAGCATCTGCCCGGCA |
| | | TTTCTGACAGCTTTGTGA |
| | | ACTGGGTGGCCGAGAAG |
| | | GAATGGGAGTTGCCGCC |
| | | AGATTCTGACATGGATC |
| | | TGAATCTGATTGAGCAG |
| | | GCACCCCTGACCGTGGC |
| | | CGAGAAGCTGCAGCGCG |
| | | ACTTTCTGACGGAATGG |
| | | CGCCGTGTGAGTAAGGC |
| | | CCCGGAGGCCCTTTTCTT |
| | | TGTGCAATTTGAGAAGG |
| | | GAGAGAGCTACTTCCAC |
| | | ATGCACGTGCTCGTGGA |
| | | AACCACCGGGGTGAAAT |
| | | CCATGGTTTTGGGACGT |
| | | TTCCTGAGTCAGATTCG |
| | | CGAAAAACTGATTCAGA |
| | | GAATTTACCGCGGGATC |
| | | GAGCCGACTTTGCCAAA |
| | | CTGGTTCGCGGTCACAA |
| | | AGACCAGAAATGGCGCC |
| | | GGAGGCGGGAACAAGG |
| | | TGGTGGATGAGTGCTAC |
| | | ATCCCCAATTACTTGCTC |
| | | CCCAAAACCCAGCCTGA |
| | | GCTCCAGTGGGCGTGGA |
| | | CTAATATGGAACAGTAT |
| | | TTAAGCGCCTGTTTGAA |
| | | TCTCACGGAGCGTAAAC |
| | | GGTTGGTGGCGCAGCAT |
| | | CTGACGCACGTGTCGCA |
| | | GACGCAGGAGCAGAAC |
| | | AAAGAGAATCAGAATCC |
| | | CAATTCTGATGCGCCGG |
| | | TGATCAGATCAAAAACT |
| | | TCAGCCAGGTACATGGA |
| | | GCTGGTCGGGTGGCTCG |
| | | TGGACAAGGTGAGTTTG |
| | | GGGACCCTTGATTGTTCT |
| | | TTCTTTTTCGCTATTGTA |
| | | AAATTCATGTTATATGG |
| | | AGGGGGCAAAGTTTTCA |
| | | GGGTGTTGTTTAGAATG |
| | | GGAAGATGTCCCTTGTA |
| | | TCACCATGGACCCTCAT |
| | | GATAATTTTGTTTCTTTC |
| | | ACTTTCTACTCTGTTGAC |
| | | AACCGTTGTCTCCTCTTA |
| | | TTTTCTTTTCATTTTCTGT |
| | | AACTTTTTCGTTAAACTT |
| | | TAGCTTGCATTTGTAAC |
| | | GAATTTTTAAATTCACTT |
| | | TTGTTTATTTGTCAGATT |
| | | GTAAGTACTTTCTCTAAT |
| | | CACTTTTTTTTCAAGGCA |
| | | ATCAGGGTATATTATAT |
| | | TGTACTTCAGCACAGTTT |
| | | TAGAGAACATAACTTCG |
| | | TATAAAGTATACTATAC |
| | | GAAGTTATCGGGCCCCT |
| | | CTGCTAACCATGTTCAT |
| | | GCCTTCTTCTTTTTCCTA |
| | | CAGATGTCAGAACTCAT |
| | | TAAAGAGAATATGCACA |
| | | TGAAGCTGTATATGGAA |
| | | GGTACTGTAGACAACCA |
| | | CCATTTCAAATGCACGT |
| | | CCGAAGGTGAGGGGAA |
| | | GCCATACGAGGGTACCC |
| | | AAACTATGCGCATCAAA |
| | | GTGGTTGAGGGTGGCCC |
| | | CCTGCCATTCGCATTCG |
| | | ACATCCTGGCAACTAGC |
| | | TTTCTTTACGGTTCCAAG |
| | | ACATTCATAAATCATAC |
| | | CCAGGGTATTCCCGATT |
| | | TCTTCAAACAATCCTTCC |
| | | CGGAAGGGTTTACTTGG |
| | | GAGCGGGTCACGACATA |
| | | TGAAGACGGGGGTGTTC |
| | | TTACAGCCACACAGGAT |
| | | ACGAGTTTGCAAGACGG |
| | | TTGTCTTATCTATAACGT |
| | | GAAGATTCGGGGTGTGA |
| | | ATTTCACATCCAATGGC |
| | | CCGGTGATGCAGAAAAA |
| | | AACACTGGGCTGGGAAG |
| | | CATTTACGGAGACGTTG |
| | | TATCCCGCCGATGGAGG |
| | | TCTCGAGGGCCGAAACG |
| | | ATATGGCCCTCAAGTTG |
| | | GTAGGTGGTTCTCACCTT |
| | | ATAGCAAACATTAAGAC |
| | | CACGTATCGATCAAAAA |
| | | AACCCGCTAAGAATCTG |
| | | AAAATGCCAGGCGTGTA |
| | | TTATGTTGATTACAGACT |
| | | GGAGCGAATAAAAGAG |
| | | GCTAACAATGAGACCTA |
| | | CGTCGAACAGCATGAAG |
| | | TCGCTGTAGCTAGATAT |
| | | TGCGACCTCCCGTCAAA |
| | | GTTGGGCCATAAATTGA |
| | | ATTAACCTCAGGTGCAG |
| | | GCTGCCTATCAGAAGGT |
| | | GGTGGCTGGTGTGGCCA |
| | | ATGCCCTGGCTCACAAA |
| | | TACCACTGAGATCTTTTT |
| | | CCCTCTGCCAAAAATTA |
| | | TGGGGACATCATGAAGC |
| | | CCCTTGAGCATCTGACTT |
| | | CTGGCTAATAAAGGAAA |
| | | TTTATTTTCATTGCAATA |
| | | GTGTGTTGGAATTTTTTG |
| | | TGTCTCTCACTCGGAAG |
| | | GACATATGGGAGGGCAA |
| | | ATCATTTAAAACATCAG |
| | | AATGAGTATTTGGTTTA |
| | | GAGTTTGGCAACATATG |
| | | CCCATATGCTGGCTGCC |
| | | ATGAACAAAGGTTGGCT |
| | | ATAAAGAGGTCATCAGT |
| | | ATATGAAACAGCCCCCT |
| | | GCTGTCCATTCCTTATTC |
| | | CATAGAAAAGCCTTGAC |
| | | TTGAGGTTAGATTTTTTT |
| | | TATATTTTGTTTTGTGTT |
| | | ATTTTTTTCTTTAACATC |
| | | CCTAAAATTTTCCTTACA |
| | | TGTTTTACTAGCCAGATT |
| | | TTTCCTCCTCTCCTGACT |
| | | ACTCCCAGTCATAGCTG |
| | | TCCCTCTTCTCTTATGGA |
| | | GATCATAACTTCGTATA |
| | | AAGTATACTATACGAAG |
| | | TTATAATTGTTATAATTA |
| | | AATGATAAGGTAGAATA |
| | | TTTCTGCATATAAATTCT |
| | | GGCTGGCGTGGAAATAT |
| | | TCTTATTGGTAGAAACA |
| | | ACTACACCCTGGTCATC |
| | | ATCCTGCCTTTCTCTTTA |
| | | TGGTTACAATGATATAC |
| | | ACTGTTTGAGATGAGGA |
| | | TAAAATACTCTGAGTCC |
| | | AAACCGGGCCCCTCTGC |
| | | TAACCATGTTCATGCCTT |
| | | CTTCTTTTTCCTACAGGG |
| | | GATTACCTCGGAGAAGC |
| | | AGTGGATCCAGGAGGAC |
| | | CAGGCCTCATACATCTC |
| | | CTTCAATGCGGCCTCCA |
| | | ACTCGCGGTCCCAAATC |
| | | AAGGCTGCCTTGGACAA |
| | | TGCGGGAAAGATTATGA |
| | | GCCTGACTAAAACCGCC |
| | | CCCGACTACCTGGTGGG |
| | | CCAGCAGCCCGTGGAGG |
| | | ACATTTCCAGCAATCGG |
| | | ATTTATAAAATTTTGGA |
| | | ACTAAACGGGTACGATC |
| | | CCCAATATGCGGCTTCC |
| | | GTCTTTCTGGGATGGGC |
| | | CACGAAAAAGTTCGGCA |
| | | AGAGGAACACCATCTGG |
| | | CTGTTTGGGCCTGCAAC |
| | | TACCGGGAAGACCAACA |
| | | TCGCGGAGGCCATAGCC |
| | | CACACTGTGCCCTTCTAC |
| | | GGGTGCGTAAACTGGAC |
| | | CAATGAGAACTTTCCCT |
| | | TCAACGACTGTGTCGAC |
| | | AAGATGGTGATCTGGTG |
| | | GGAGGAGGGGAAGATG |
| | | ACCGCCAAGGTCGTGGA |
| | | GTCGGCCAAAGCCATTC |
| | | TCGGAGGAAGCAAGGTG |
| | | CGCGTGGACCAGAAATG |
| | | CAAGTCCTCGGCCCAGA |
| | | TAGACCCGACTCCCGTG |
| | | ATCGTCACCTCCAACAC |
| | | CAACATGTGCGCCGTGA |
| | | TTGACGGGAACTCAACG |
| | | ACCTTCGAACACCAGCA |
| | | GCCGTTGCAAGACCGGA |
| | | TGTTCAAATTTGAACTC |
| | | ACCCGCCGTCTGGATCA |
| | | TGACTTTGGGAAGGTCA |
| | | CCAAGCAGGAAGTCAAA |
| | | GACTTTTTCCGGTGGGC |
| | | AAAGGATCACGTGGTTG |
| | | AGGTGGAGCATGAATTC |
| | | TACGTCAAAAAGGGTGG |
| | | AGCCAAGAAAAGACCCG |
| | | CCCCCAGTGACGCAGAT |
| | | ATAAGTGAGCCCAAACG |
| | | GGTGCGCGAGTCAGTTG |
| | | CGCAGCCATCGACGTCA |
| | | GACGCGGAAGCTTCGAT |
| | | CAACTACGCAGACAGGT |
| | | ACCAAAACAAATGTTCT |
| | | CGTCACGTGGGCATGAA |
| | | TCTGATGCTGTTTCCCTG |
| | | CAGACAATGCGAGAGAA |
| | | TGAATCAGAATTCAAAT |
| | | ATCTGCTTCACTCACGG |
| | | ACAGAAAGACTGTTTAG |
| | | AGTGCTTTCCCGTGTCA |
| | | GAATCTCAACCCGTTTCT |
| | | GTCGTCAAAAAGGCGTA |
| | | TCAGAAACTGTGCTACA |
| | | TTCATCATATCATGGGA |
| | | AAGGTGCCAGACGCTTG |
| | | CACTGCCTGCGATCTGG |
| | | TCAATGTGGATTTGGAT |
| | | GACTGCATCTTTGAACA |
| | | ATAAATGATTTGTAAAT |
| | | AAATTTAGTAGTCATGT |
| | | CTTTTGTTGATCACCCTC |
| | | CAGATTGGTTGGAAGAA |
| | | GTTGGTGAAGGTCTTCG |
| | | CGAGTTTTTGGGCCTTG |
| | | AAGCGGGCCCACCGAAA |
| | | CCAAAACCCAATCAGCA |
| | | GCATCAAGATCAAGCCC |
| | | GTGGTCTTGTGCTGCCTG |
| | | GTTATAACTATCTCGGA |
| | | CCCGGAAACGGTCTCGA |
| | | TCGAGGAGAGCCTGTCA |
| | | ACAGGGCAGACGAGGTC |
| | | GCGCGAGAGCACGACAT |
| | | CTCGTACAACGAGCAGC |
| | | TTGAGGCGGGAGACAAC |
| | | CCCTACCTCAAGTACAA |
| | | CCACGCGGACGCCGAGT |
| | | TTCAGGAGAAGCTCGCC |
| | | GACGACACATCCTTCGG |
| | | GGGAAACCTCGGAAAGG |
| | | CAGTCTTTCAGGCCAAG |
| | | AAAAGGGTTCTCGAACC |
| | | TTTTGGCCTGGTTGAAG |
| | | AGGGTGCTAAGACGGCC |
| | | CCTACCGGAAAGCGGAT |
| | | AGACGACCACTTTCCAA |
| | | AAAGAAAGAAGGCTCG |
| | | GACCGAAGAGGACTCCA |
| | | AGCCTTCCACCTCGTCA |
| | | GACGCCGAAGCTGGACC |
| | | CAGCGGATCCCAGCAGC |
| | | TGCAAATCCCAGCCCAA |
| | | CCAGCCTCAAGTTTGGG |
| | | AGCTGATACAATGTCTG |
| | | CGGGAGGTGGCGGCCCA |
| | | TTGGGCGACAATAACCA |
| | | AGGTGCCGATGGAGTGG |
| | | GCAATGCCTCGGGAGAT |
| | | TGGCATTGCGATTCCAC |
| | | GTGGATGGGGGACAGAG |
| | | TCGTCACCAAGTCCACC |
| | | CGAACCTGGGTGCTGCC |
| | | CAGCTACAACAACCACC |
| | | AGTACCGAGAGATCAAA |
| | | AGCGGCTCCGTCGACGG |
| | | AAGCAACGCCAACGCCT |
| | | ACTTTGGATACAGCACC |
| | | CCCTGGGGGTACTTTGA |
| | | CTTTAACCGCTTCCACA |
| | | GCCACTGGAGCCCCCGA |
| | | GACTGGCAAAGACTCAT |
| | | CAACAACTACTGGGGCT |
| | | TCAGACCCCGGTCCCTC |
| | | AGAGTCAAAATCTTCAA |
| | | CATTCAAGTCAAAGAGG |
| | | TCACGGTGCAGGACTCC |
| | | ACCACCACCATCGCCAA |
| | | CAACCTCACCTCCACCG |
| | | TCCAAGTGTTTACGGAC |
| | | GACGACTACCAGCTGCC |
| | | CTACGTCGTCGGCAACG |
| | | GGACCGAGGGATGCCTG |
| | | CCGGCCTTCCCTCCGCA |
| | | GGTCTTTACGCTGCCGC |
| | | AGTACGGTTACGCGACG |
| | | CTGAACCGCGACAACAC |
| | | AGAAAATCCCACCGAGA |
| | | GGAGCAGCTTCTTCTGC |
| | | CTAGAGTACTTTCCCAG |
| | | CAAGATGCTGAGAACGG |
| | | GCAACAACTTTGAGTTT |
| | | ACCTACAACTTTGAGGA |
| | | GGTGCCCTTCCACTCCA |
| | | GCTTCGCTCCCAGTCAG |
| | | AACCTGTTCAAGCTGGC |
| | | CAACCCGCTGGTGGACC |
| | | AGTACTTGTACCGCTTC |
| | | GTGAGCACAAATAACAC |
| | | TGGCGGAGTCCAGTTCA |
| | | ACAAGAACCTGGCCGGG |
| | | AGATACGCCAACACCTA |
| | | CAAAAACTGGTTCCCGG |
| | | GGCCCATGGGCCGAACC |
| | | CAGGGCTGGAACCTGGG |
| | | CTCCGGGGTCAACCGCG |
| | | CCAGTGTCAGCGCCTTC |
| | | GCCACGACCAATAGGAT |
| | | GGAGCTCGAGGGCGCGA |
| | | GTTACCAGGTGCCCCCG |
| | | CAGCCGAACGGCATGAC |
| | | CAACAACCTCCAGGGCA |
| | | GCAACACCTATGCCCTG |
| | | GAGAACACTATGATCTT |
| | | CAACAGCCAGCCGGCGA |
| | | ACCCGGGCACCACCGCC |
| | | ACGTACCTCGAGGGCAA |
| | | CATGCTCATCACCAGCG |
| | | AGAGCGAGACGCAGCCG |
| | | GTGAACCGCGTGGCGTA |
| | | CAACGTCGGCGGGCAGA |
| | | TGGCCACCAACAACCAG |
| | | AGCTCCACCACTGCCCC |
| | | CGCGACCGGCACGTACA |
| | | ACCTCCAGGAAATCGTG |
| | | CCCGGCAGCGTGTGGAT |
| | | GGAGAGGGACGTGTACC |
| | | TCCAAGGACCCATCTGG |
| | | GCCAAGATCCCAGAGAC |
| | | GGGGGCGCACTTTCACC |
| | | CCTCTCCGGCCATGGGC |
| | | GGATTCGGACTCAAACA |
| | | CCCACCGCCCATGATGC |
| | | TCATCAAGAACACGCCT |
| | | GTGCCCGGAAATATCAC |
| | | CAGCTTCTCGGACGTGC |
| | | CCGTCAGCAGCTTCATC |
| | | ACCCAGTACAGCACCGG |
| | | GCAGGTCACCGTGGAGA |
| | | TGGAGTGGGAGCTCAAG |
| | | AAGGAAAACTCCAAGAG |
| | | GTGGAACCCAGAGATCC |
| | | AGTACACAAACAACTAC |
| | | AACGACCCCCAGTTTGT |
| | | GGACTTTGCCCCGGACA |
| | | GCACCGGGGAATACAGA |
| | | ACCACCAGACCTATCGG |
| | | AACCCGATACCTTACCC |
| | | GACCCCTTTAATTGCTTG |
| | | TTAATCAATAAACCGTT |
| | | TAATTCGTTTCAGTTGAA |
| | | CTTTGGTCTCTGCGTATT |
| | | TCTTTCTTATCTAGTTTC |
| | | CATGGCTACGTAGATAA |
| | | GTAGCATGGCGGGTTAA |
| | | TCATTAACTACAGCCCG |
| | | GGCGTTTAAACAGCGGG |
| | | CGGAGGGGTGGAGTCGT |
| | | GACGTGAATTACGTCAT |
| | | AGGGTTAGGGAGGTCCT |
| | | GTATTAGAGGTCACGTG |
| | | AGTGTTTTGCGACATTTT |
| | | GCGACACCATGT |
| SEQ ID NO: 48 | Helper Construct (E2A; | GCCTCCACGGCCACTAGTC |
| | E4orf6; VA RNA (G16A; | CATAGAGCCCACCGCATCC |
| | G60A)) | CCAGCATGCCTGCTATTGT |
| | | CTTCCCAATCCTCCCCCTT |
| | | GCTGTCCTGCCCCACCCCA |
| | | CCCCCTAGAATAGAATGAC |
| | | ACCTACTCAGACAATGCGA |
| | | TGCAATTTCCTCATTTTATT |
| | | AGGAAAGGACAGTGGGAG |
| | | TGGCACCTTCCAGGGTCAA |
| | | GGAAGGCACGGGGGAGGG |
| | | GCAAACAACAGATGGCTG |
| | | GCAACTAGAAGGCACAGC |
| | | TACATGGGGGTAGAGTCAT |
| | | AATCGTGCATCAGGATAGG |
| | | GCGGTGGTGCTGCAGCAGC |
| | | GCGCGAATAAACTGCTGCC |
| | | GCCGCCGCTCCGTCCTGCA |
| | | GGAATACAACATGGCAGT |
| | | GGTCTCCTCAGCGATGATT |
| | | CGCACCGCCCGCAGCATGA |
| | | GACGCCTTGTCCTCCGGGC |
| | | ACAGCAGCGCACCCTGATC |
| | | TCACTTAAATCAGCACAGT |
| | | AACTGCAGCACAGCACCA |
| | | CAATATTGTTCAAAATCCC |
| | | ACAGTGCAAGGCGCTGTAT |
| | | CCAAAGCTCATGGCGGGG |
| | | ACCACAGAACCCACGTGG |
| | | CCATCATACCACAAGCGCA |
| | | GGTAGATTAAGTGGCGACC |
| | | CCTCATAAACACGCTGGAC |
| | | ATAAACATTACCTCTTTTG |
| | | GCATGTTGTAATTCACCAC |
| | | CTCCCGGTACCATATAAAC |
| | | CTCTGATTAAACATGGCGC |
| | | CATCCACCACCATCCTAAA |
| | | CCAGCTGGCCAAAACCTGC |
| | | CCGCCGGCTATGCACTGCA |
| | | GGGAACCGGGACTGGAAC |
| | | AATGACAGTGGAGAGCCC |
| | | AGGACTCGTAACCATGGAT |
| | | CATCATGCTCGTCATGATA |
| | | TCAATGTTGGCACAACACA |
| | | GGCACACGTGCATACACTT |
| | | CCTCAGGATTACAAGCTCC |
| | | TCCCGCGTCAGAACCATAT |
| | | CCCAGGGAACAACCCATTC |
| | | CTGAATCAGCGTAAATCCC |
| | | ACACTGCAGGGAAGACCT |
| | | CGCACGTAACTCACGTTGT |
| | | GCATTGTCAAAGTGTTACA |
| | | TTCGGGCAGCAGCGGATG |
| | | ATCCTCCAGTATGGTAGCG |
| | | CGGGTCTCTGTCTCAAAAG |
| | | GAGGTAGGCGATCCCTACT |
| | | GTACGGAGTGCGCCGAGA |
| | | CAACCGAGATCGTGTTGGT |
| | | CGTAGTGTCATGCCAAATG |
| | | GAACGCCGGACGTAGTCAT |
| | | GGTTGTGGCCATATTATCA |
| | | TCGTGTTTTTCAAAGGAAA |
| | | ACCACGTCCCCGTGGTTCG |
| | | GGGGGCCTAGACGTTTTTT |
| | | TAACCTCGACTAAACACAT |
| | | GTAAAGCATGTGCACCGA |
| | | GGCCCCAGATCAGATCCCA |
| | | TACAATGGGGTACCTTCTG |
| | | GGCATCCTTCAGCCCCTTG |
| | | TTGAATACGCTTGAGGAGA |
| | | GCCATTTGACTCTTTCCAC |
| | | AACTATCCAACTCACAACG |
| | | TGGCACTGGGGTTGTGCCG |
| | | CCTTTGCAGGTGTATCTTA |
| | | TACACGTGGCTTTTGGCCG |
| | | CAGAGGCACCTGTCGCCAG |
| | | GTGGGGGGTTCCGCTGCCT |
| | | GCAAAGGGTCGCTACAGA |
| | | CGTTGTTTGTCTTCAAGAA |
| | | GCTTCCAGAGGAACTGCTT |
| | | CCTTCACGACATTCAACAG |
| | | ACCTTGCATTCCTTTGGCG |
| | | AGAGGGGAAAGACCCCTA |
| | | GGAATGCTCGTCAAGAAG |
| | | ACAGGGCCAGGTTTCCGGG |
| | | CCCTCACATTGCCAAAAGA |
| | | CGGCAATATGGTGGAAAA |
| | | TAACATATAGACAAACGC |
| | | ACACCGGCCTTATTCCAAG |
| | | CGGCTTCGGCCAGTAACGT |
| | | TAGGGGGGGGGGAGGGAG |
| | | AGGGGCTTAAAAATCAAA |
| | | GGGGTTCTGCCGCGCATCA |
| | | CTATGCGCCACTGGCAGGG |
| | | ACACGTTGCGATACTGGTG |
| | | TTTAGTGCTCCACTTAAAC |
| | | TCAGGCACAACCATCCGCG |
| | | GCAGCTCGGTGAAGTTTTC |
| | | ACTCCACAGGCTGCGCACC |
| | | ATCACCAACGCGTTTAGCA |
| | | GGTCGGGCGCCGATATCTT |
| | | GAAGTCGCAGTTGGGGCCT |
| | | CCGCCCTGCGCGCGCGAGT |
| | | TGCGATACACAGGGTTGCA |
| | | GCACTGGAACACTATCAGC |
| | | GCCGGGTGGTGCACGCTGG |
| | | CCAGCACGCTCTTGTCGGA |
| | | GATCAGATCCGCGTCCAGG |
| | | TCCTCCGCGTTGCTCAGGG |
| | | CGAACGGAGTCAACTTTGG |
| | | TAGCTGCCTTCCCAAAAAG |
| | | GGTGCATGCCCAGGCTTTG |
| | | AGTTGCACTCGCACCGTAG |
| | | TGGCATCAGAAGGTGACC |
| | | GTGCCCGGTCTGGGCGTTA |
| | | GGATACAGCGCCTGCATGA |
| | | AAGCCTTGATCTGCTTAAA |
| | | AGCCACCTGAGCCTTTGCG |
| | | CCTTCAGAGAAGAACATGC |
| | | CGCAAGACTTGCCGGAAA |
| | | ACTGATTGGCCGGACAGGC |
| | | CGCGTCATGCACGCAGCAC |
| | | CTTGCGTCGGTGTTGGAGA |
| | | TCTGCACCACATTTCGGCC |
| | | CCACCGGTTCTTCACGATC |
| | | TTGGCCTTGCTAGACTGCT |
| | | CCTTCAGCGCGCGCTGCCC |
| | | GTTTTCGCTCGTCACATCC |
| | | ATTTCAATCACGTGCTCCT |
| | | TATTTATCATAATGCTCCC |
| | | GTGTAGACACTTAAGCTCG |
| | | CCTTCGATCTCAGCGCAGC |
| | | GGTGCAGCCACAACGCGC |
| | | AGCCCGTGGGCTCGTGGTG |
| | | CTTGTAGGTTACCTCTGCA |
| | | AACGACTGCAGGTACGCCT |
| | | GCAGGAATCGCCCCATCAT |
| | | CGTCACAAAGGTCTTGTTG |
| | | CTGGTGAAGGTCAGCTGCA |
| | | ACCCGCGGTGCTCCTCGTT |
| | | TAGCCAGGTCTTGCATACG |
| | | GCCGCCAGAGCTTCCACTT |
| | | GGTCAGGCAGTAGCTTGAA |
| | | GTTTGCCTTTAGATCGTTA |
| | | TCCACGTGGTACTTGTCCA |
| | | TCAACGCGCGCGCAGCCTC |
| | | CATGCCCTTCTCCCACGCA |
| | | GACACGATCGGCAGGCTC |
| | | AGCGGGTTTATCACCGTGC |
| | | TTTCACTTTCCGCTTCACTG |
| | | GACTCTTCCTTTTCCTCTTG |
| | | CGTCCGCATACCCCGCGCC |
| | | ACTGGGTCGTCTTCATTCA |
| | | GCCGCCGCACCGTGCGCTT |
| | | ACCTCCCTTGCCGTGCTTG |
| | | ATTAGCACCGGTGGGTTGC |
| | | TGAAACCCACCATTTGTAG |
| | | CGCCACATCTTCTCTTTCTT |
| | | CCTCGCTGTCCACGATCAC |
| | | CTCTGGGGATGGCGGGCGC |
| | | TCGGGCTTGGGAGAGGGG |
| | | CGCTTCTTTTTCTTTTTGGA |
| | | CGCAATGGCCAAATCCGCC |
| | | GTCGAGGTCGATGGCCGCG |
| | | GGCTGGGTGTGCGCGGCAC |
| | | CAGCGCATCTTGTGACGAG |
| | | TCTTCTTCGTCCTCGGACTC |
| | | GAGACGCCGCCTCAGCCGC |
| | | TTTTTTGGGGGCGCGCGCT |
| | | TGTCGTCATCGTCTTTGTA |
| | | GTCGGGAGGCGGCGGCGA |
| | | CGGCGACGGGGACGACAC |
| | | GTCCTCCATGGTTGGTGGA |
| | | CGTCGCGCCGCACCGCGTC |
| | | CGCGCTCGGGGGTGGTTTC |
| | | GCGCTGCTCCTCTTCCCGA |
| | | CTGGCCATGGTGGCCGAGG |
| | | ATAACTTCGTATATGGTTT |
| | | CTTATACGAAGTTATGATC |
| | | CAGACATGATAAGATACAT |
| | | TGATGAGTTTGGACAAACC |
| | | ACAACTAGAATGCAGTGA |
| | | AAAAAATGCTTTATTTGTG |
| | | AAATTTGTGATGCTATTGC |
| | | TTTATTTGTAACCATTATA |
| | | AGCTGCAATAAACAAGTTA |
| | | ACAACAACAATTGCATTCA |
| | | TTTTATGTTTCAGGTTCAG |
| | | GGGGAGGTGTGGGAGGTT |
| | | TTTTAAAGCAAGTAAAACC |
| | | TCTACAAATGTGGTATGGC |
| | | TGATTATGATCCTCTAGAG |
| | | TCGCAGATCTGCTACGTAT |
| | | CAAGCTGTGGCAGGGAAA |
| | | CCCTCTGCCTCCCCCGTGA |
| | | TGTAATACTTTTGCAAGGA |
| | | ATGCGATGAAGTAGAGCC |
| | | CGCAGTGGCCAAGTGGCTT |
| | | TGGTCCGTCTCCTCCACGG |
| | | ATGCCCCTCCACGGCTAGT |
| | | GGGCGCATGTAGGCGGTG |
| | | GGCGTCCGCCGCCTCCAGC |
| | | AGCAGGTCATAGAGGGGC |
| | | ACCACGTTCTTGCACTTCA |
| | | TGCTGTACAGATGCTCCAT |
| | | GCCTTTGTTACTCATGTGT |
| | | CGGATGTGGGAGAGGATG |
| | | AGGAGGAGCTGGGCCAGC |
| | | CGCTGGTGCTGCTGCTGCA |
| | | GGGTCAGGCCTGCCTTGGC |
| | | CATCAGGTGGATCAAAGTG |
| | | TCTGTGATCTTGTCCAGGA |
| | | CTCGGTGGATATGGTCCTT |
| | | CTCTTCCAGAGACTTCAGG |
| | | GTGCTGGACAGAAATGTGT |
| | | ACACTCCAGAATTAAGCAA |
| | | AATAATAGATTTGAGGCAC |
| | | ACAAACTCCTCTCCCTGCA |
| | | GATTCATCATGCGGAACCG |
| | | AGATGATGTAGCCAGCAG |
| | | CATGTCGAAGATCTCCACC |
| | | ATGCCCTCTACACATTTTC |
| | | CCTGGTTCCTGTCCAAGAG |
| | | CAAGTTAGGAGCAAACAG |
| | | TAGCTTCACTGGGTGCTCC |
| | | ATGGAGCGCCAGACGAGA |
| | | CCAATCATCAGGATCTCTA |
| | | GCCAGGCACATTCTAGAAG |
| | | GTGGACCTGATCATGGAGG |
| | | GTCAAATCCACAAAGCCTG |
| | | GCACCCTCTTCGCCCAGTT |
| | | GATCATGTGAACCAGCTCC |
| | | CTGTCTGCCAGGTTGGTCA |
| | | GTAAGCCCATCATCGAAGC |
| | | TTCACTGAAGGGTCTGGTA |
| | | GGATCATACTCGGAATAGA |
| | | GTATGGGGGGCTCAGCATC |
| | | CAACAAGGCACTGACCATC |
| | | TGGTCGGCCGTCAGGGACA |
| | | AGGCCAGGCTGTTCTTCTT |
| | | AGAGCGTTTGATCATGAGC |
| | | GGGCTTGGCCAAAGGTTGG |
| | | CAGCTCTCATGTCTCCAGC |
| | | AGATGGCTCGAGATCGCCA |
| | | TCTTCCAGCAGGCGCACCA |
| | | TTGCCCCTGTTTCACTATCC |
| | | AGGTTACGGATATAGTTCA |
| | | TGACAATATTTACATTGGT |
| | | CCAGCCACCAGCTTGCATG |
| | | ATCTCCGGTATTGAAACTC |
| | | CAGCGCGGGCCATATCTCG |
| | | CGCGGCTCCGACACGGGC |
| | | ACTGTGTCCAGACCAGGCC |
| | | AGGTATCTCTGACCAGAGT |
| | | CATCCTAAAATACACAAAC |
| | | AATTAGAATCAGTAGTTTA |
| | | ACACATTATACACTTAAAA |
| | | ATTTTATATTTACCTTAGC |
| | | GCCGTAAATCAATCGATGA |
| | | GTTGCTTCAAAAATCCCTT |
| | | CCAGGGCGCGAGTTGATA |
| | | GCTGGCTGGTGGCAGATGG |
| | | CGCGGCAACACCATTTTTT |
| | | CTGACCCGGCAAAACAGG |
| | | TAGTTATTCGGATCATCAG |
| | | CTACACCAGAGACGGAAA |
| | | TCCATCGCTCGACCAGTTT |
| | | AGTGACTCCCAGGCTAAGT |
| | | GCCTTCTCTACACCTGCGG |
| | | TGCTAACCAGCGTTTTCGT |
| | | TCTGCCAATATGGATTAAC |
| | | ATTCTCCCACCGTCAGTAC |
| | | GTGAGATATCTTTAACCCT |
| | | GATCCTGGCAATTTCGGCT |
| | | ATACGTAACAGGGTGTTAT |
| | | AAGCAATCCCCAGAAATG |
| | | CCAGATTACGTATATCCTG |
| | | GCAGCGATCGCTATTTTCC |
| | | ATGAGTGAACGGACTTGGT |
| | | CGAAATCAGTGCGTTCGAA |
| | | CGCTAGAGCCTGTTTTGCA |
| | | CGTTCACCGGCATCAACGT |
| | | TTTCTTTTCGGATCCGCCG |
| | | CATAACCAGTGAAACAGC |
| | | ATTGCTGTCACTTGGTCGT |
| | | GGCAGCCCGGACCGACGA |
| | | TGAAGCATGTTTAGCTGGC |
| | | CCAAATGTTGCTGGATAGT |
| | | TTTTACTGCCAGACCGCGC |
| | | GCTTGAAGATATAGAAGAT |
| | | AATCGCGAACATCTTCAGG |
| | | TTCTGCGGGAAACCATTTC |
| | | CGGTTATTCAACTTGCACC |
| | | ATGCCGCCCACGACCGGCA |
| | | AACGGACAGAAGCATTTTC |
| | | CAGGTATGCTCAGAAAAC |
| | | GCCTGGCGATCCCTGAACA |
| | | TGTCCATCAGGTTCTTGCG |
| | | AACCTCATCACTCGTTGCA |
| | | TCGACCGGTAATGCAGGCA |
| | | AATTTTGGTGTACGGTCAG |
| | | TAAATTGGACATGGTGGCT |
| | | ACGTAATAACTTCGTATAT |
| | | GGTTTCTTATACGAAGTTA |
| | | TGCGGCCGCTTTACGAGGG |
| | | TAGGAAGTGGTACGGAAA |
| | | GTTGGTATAAGACAAAAGT |
| | | GTTGTGGAATTGCTCCAGG |
| | | CGATCTGACGGTTCACTAA |
| | | ACGAGCTCTGCTTTTATAG |
| | | GCGCCCACCGTACACGCCT |
| | | AAAGCTTATACGTTCTCTA |
| | | TCACTGATAGGGAGTAAAC |
| | | TGGATATACGTTCTCTATC |
| | | ACTGATAGGGAGTAAACT |
| | | GTAGATACGTTCTCTATCA |
| | | CTGATAGGGAGTAAACTG |
| | | GTCATACGTTCTCTATCAC |
| | | TGATAGGGAGTAAACTCCT |
| | | TATACGTTCTCTATCACTG |
| | | ATAGGGAGTAAAGTCTGC |
| | | ATACGTTCTCTATCACTGA |
| | | TAGGGAGTAAACTCTTCAT |
| | | ACGTTCTCTATCACTGATA |
| | | GGGAGTAAACTCGCGGCC |
| | | GCAGAGAAATGTTCTGGCA |
| | | CCTGCACTTGCACTGGGGA |
| | | CAGCCTATTTTGCTAGTTT |
| | | GTTTTGTTTCGTTTTGTTTT |
| | | GATGGAGAGCGTATGTTAG |
| | | TACTATCGATTCACACAAA |
| | | AAACCAACACACAGATGT |
| | | AATGAAAATAAAGATATTT |
| | | TATTGGATCTGCGATCGCT |
| | | CCGGTGCCCGTCAGTGGGC |
| | | AGAGCGCACATCGCCCAC |
| | | AGTCCCCGAGAAGTTGGG |
| | | GGGAGGGGTCGGCAATTG |
| | | AACGGGTGCCTAGAGAAG |
| | | GTGGCGCGGGGTAAACTG |
| | | GGAAAGTGATGTCGTGTAC |
| | | TGGCTCCGCCTTTTTCCCG |
| | | AGGGTGGGGGAGAACCGT |
| | | ATGTAAGTGCAGTAGTCGC |
| | | CGTGAACGTTCTTTTTCGC |
| | | AACGGGTTTGCCGCCAGAA |
| | | CACAGCTGAAGCTTCGAGG |
| | | GGCTCGCATCTCTCCTTCA |
| | | CGCGCCCGCCGCCCTACCT |
| | | GAGGCCGCCATCCACGCCG |
| | | GTTGAGTCGCGTTCTGCCG |
| | | CCTCCCGCCTGTGGTGCCT |
| | | CCTGAACTGCGTCCGCCGT |
| | | CTAGGTAAGTTTAAAGCTC |
| | | AGGTCGAGACCGGGCCTTT |
| | | GTCCGGCGCTCCCTTGGAG |
| | | CCTACCTAGACTCAGCCGG |
| | | CTCTCCACGCTTTGCCTGA |
| | | CCCTGCTTGCTCAACTCTA |
| | | CGTCTTTGTTTCGTTTTCTG |
| | | TTCTGCGCCGTTACAGATC |
| | | CAAGCTGTGACCGGCGCCT |
| | | ACGCTAGCGGATCCGCCGC |
| | | CACCATGTCTAGACTGGAC |
| | | AAGAGCAAAGTCATAAAC |
| | | TCTGCTCTGGAATTACTCA |
| | | ATGGAGTCGGTATCGAAG |
| | | GCCTGACGACAAGGAAAC |
| | | TCGCTCAAAAGCTGGGAGT |
| | | TGAGCAGCCTACCCTGTAC |
| | | TGGCACGTGAAGAACAAG |
| | | CGGGCCCTGCTCGATGCCC |
| | | TGCCAATCGAGATGCTGGA |
| | | CAGGCATCATACCCACTCC |
| | | TGCCCCCTGGAAGGCGAGT |
| | | CATGGCAAGACTTTCTGCG |
| | | GAACAACGCCAAGTCATA |
| | | CCGCTGTGCTCTTCTCTCA |
| | | CATCGCGACGGGGCTAAA |
| | | GTGCATCTCGGCACCCGCC |
| | | CAACAGAGAAACAGTACG |
| | | AAACCCTGGAAAATCAGCT |
| | | CGCGTTCCTGTGTCAGCAA |
| | | GGCTTCTCCCTGGAGAACG |
| | | CACTGTACGCTCTGTCCGC |
| | | CGTGGGCCACTTTACACTG |
| | | GGCTGCGTATTGGAGGAAC |
| | | AGGAGCATCAAGTAGCAA |
| | | AAGAGGAAAGAGAGACAC |
| | | CTACCACCGATTCTATGCC |
| | | CCCACTTCTGAAACAAGCA |
| | | ATTGAGCTGTTCGACCGGC |
| | | AGGGAGCCGAACCTGCCTT |
| | | CCTTTTCGGCCTGGAACTA |
| | | ATCATATGTGGCCTGGAGA |
| | | AACAGCTAAAGTGCGAAA |
| | | GCGGCGGGCCGACCGACG |
| | | CCCTTGACGATTTTGACTT |
| | | AGACATGCTCCCAGCCGAT |
| | | GCCCTTGACGACTTTGACC |
| | | TTGATATGCTGCCTGCTGA |
| | | CGCTCTTGACGATTTTGAC |
| | | CTTGACATGCTCCCCGGGT |
| | | GAACCGGTCGCTGATCAGC |
| | | CTCGACTGTGCCTTCTAGT |
| | | TGCCAGCCATCTGTTGTTT |
| | | GCCCCTCCCCCGTGCCTTC |
| | | CTTGACCCTGGAAGGTGCC |
| | | ACTCCCACTGTCCTTTCCT |
| | | AATAAAATGAGGAAATTG |
| | | CATCGCATTGTCTGAGTAG |
| | | GTGTCATTCTATTCTGGGG |
| | | GGTGGGGTGGGGCAGGAC |
| | | AGCAAGGGGGAGGATTGG |
| | | GAAGACAATAGCAGGCAT |
| | | GCTGGGGATGCGGTGGGCT |
| | | CTATGGCTTCTGAGGCGGA |
| | | AAGAACCAGCTGGGGCTC |
| | | GACTAGAGCTTGCGGAACC |
| | | CTTAGAGGGCCTATTTCCC |
| | | ATGATTCCTTCATATTTGC |
| | | ATATACGATACAAGGCTGT |
| | | TAGAGAGATAATTAGAATT |
| | | AATTTGACTGTAAACACAA |
| | | AGATATTAGTACAAAATAA |
| | | TAACTTCGTATAATGTATG |
| | | CTATACGAAGTTATCAGAC |
| | | ATGATAAGATACATTGATG |
| | | AGTTTGGACAAACCACAAC |
| | | TAGAATGCAGTGAAAAAA |
| | | ATGCTTTATTTGTGAAATT |
| | | TGTGATGCTATTGCTTTATT |
| | | TGTAACCATTATAAGCTGC |
| | | AATAAACAAGGTACCTCA |
| | | AGCGCCGGGTTTTCGCGTC |
| | | ATGCACCACGTCCGTGGGC |
| | | CCTCGGGTACTTCAACGTC |
| | | AGCAGTAACTGTAAATCCG |
| | | AGCCGTTCATAGAAGGGC |
| | | AAATTCCTTGGCGCTGACG |
| | | TTTCAAGAAAGGCTGGCAC |
| | | TCCGGCTCGTTCTGCGGCT |
| | | TCTACTCCGGGCAATACCA |
| | | CCGCGGAACCAAGGCCCTT |
| | | TCCCTGATGATCGGGGCTA |
| | | ACGCCCACAGTAGCGAGG |
| | | AACCAAGCTGGTTCTTTAG |
| | | GGCGGTGAGGGGCGAGGA |
| | | GTCCTTCCATTTGTTGCTG |
| | | AGCCGCGAGACGAGAGCC |
| | | ACTAAGCTCAGCCATTCGG |
| | | GGACCAATTTCTGCAAATA |
| | | CAGCCCCGGCCTCAACGCT |
| | | CTCCGGAGTCGTCCACACT |
| | | GCCACTGCAGCCCCGTCGT |
| | | CGGCGACCCAAACTTTACC |
| | | GATGTCCAATCCTACCCTG |
| | | GTCAAAAAAAGTTCTTGCA |
| | | ATTCTGTAACCCGTTCAAT |
| | | ATGTCTATCAGGATCAACT |
| | | GTGTGGCGTGTAGCGGGAT |
| | | AATCCGCGAAAGCGGCAG |
| | | CCAATGTTCTCACGGCCCT |
| | | AGGGACGTCGTCTCGAGTT |
| | | GCCAGTCTGACAGTAGGTT |
| | | TATATTCTGTCATAGGTCC |
| | | AGGGTTCTCCTCCACGTCT |
| | | CCAGCCTGCTTCAGCAGGC |
| | | TGAAGTTAGTAGCTCCGGA |
| | | TCCTTTACCTCCATCACCA |
| | | GCGCCACCAGTAGAGTATC |
| | | TGGCCACAGCCACCTCGTG |
| | | CTGCTCGACGTAGGTCTCA |
| | | TCGTCGGCCTCCTTGATTC |
| | | TTTCCAGTCTGTGGTCCAC |
| | | GTTGTAGACGCCGGGCATC |
| | | TTGAGGTTCGTAGCGGGTT |
| | | TCTTGGATCTGTATGTGGT |
| | | CTCAAGGTTGCAGATCAGG |
| | | TGGCCCCCGCCCACGAGCT |
| | | TCAGGGCCATGTCACATGC |
| | | GCCTTCCAGGCCGCCGTCA |
| | | GCGGGGTACATCGTCTCGG |
| | | TGGAGGCCTCCCAGCCGAG |
| | | TGTTTTCTTCTGCATCACA |
| | | GGGCCGTTGGCTGGGAAGT |
| | | TCACCCCTCTAACCTTGAC |
| | | GTTGTAGATGAGGCAGCCG |
| | | TCCTGGAGGCTGGTGTCCT |
| | | GGGTAGCGGTCAGCACGC |
| | | CCCCGTCTTCGTATGTGGT |
| | | GACTCTCTCCCATGTGAAG |
| | | CCCTCAGGGAAGGACTGCT |
| | | TAAAGAAGTCGGGGATGC |
| | | CCGGAGGGTGCTTGATGAA |
| | | GGTTCTGCTGCCGTACATG |
| | | AAGCTGGTAGCCAGGATGT |
| | | CGAAGGCGAAGGGGAGAG |
| | | GGCCGCCCTCGACGACCTT |
| | | GATTCTCATGGTCTGGGTG |
| | | CCCTCGTAGGGCTTGCCTT |
| | | CGCCCTCGGATGTGCACTT |
| | | GAAGTGGTGGTTGTTCACG |
| | | GTGCCCTCCATGTACAGCT |
| | | TCATGGGCATGTTCTCCTT |
| | | AATCAGCTCGCTCACGGTG |
| | | GCGGCGAATTCCGAAAGG |
| | | CCCGGAGATGAGGAAGAG |
| | | GAGAACAGCGCGGCAGAC |
| | | GTGCGCTTTTGAAGCGTGC |
| | | AGAATGCCGGGCCTCCGG |
| | | AGGACCTTCGGGCGCCCGC |
| | | CCCGCCCCTGAGCCCGCCC |
| | | CTGAGCCCGCCCCCGGACC |
| | | CACCCCTTCCCAGCCTCTG |
| | | AGCCCAGAAAGCGAAGGA |
| | | GCAAAGCTGCTATTGGCCG |
| | | CTGCCCCAAAGGCCTACCC |
| | | GCTTCCATTGCTCAGCGGT |
| | | GCTGTCCATCTGCACGAGA |
| | | CTAGTGAGACGTGCTACTT |
| | | CCATTTGTCACGTCCTGCA |
| | | CGACGCGAGCTGCGGGGC |
| | | GGGGGGGAACTTCCTGACT |
| | | AGGGGAGGAGTAGAAGGT |
| | | GGCGCGAAGGGGCCACCA |
| | | AAGAACGGAGCCGGTTGG |
| | | CGCCTACCGGTGGATGTGG |
| | | AATGTGTGCGAGGCCAGA |
| | | GGCCACTTGTGTAGCGCCA |
| | | AGTGCCCAGCGGGGCTGCT |
| | | AAAGCGCATGCTCCAGACT |
| | | GCCTTGGGAAAAGCGCTCC |
| | | CCTACCCATAACTTCGTAT |
| | | AATGTATGCTATACGAAGT |
| | | TATTTTGCAGTTTTAAAAT |
| | | TATGTTTTAAAATGGACTA |
| | | TCATATGCTTACCGTAACT |
| | | TGAAAGTATTTCGATTTCT |
| | | TGGCTTTATATATCTTGTG |
| | | GAAAGGACGAAACACCGG |
| | | GCACTCTTCCGTGATCTGG |
| | | TGGATAAATTCGCAAGGGT |
| | | ATCATGGCGGACGACCGG |
| | | GATTCGAACCCCGGATCCG |
| | | GCCGTCCGCCGTGATCCAT |
| | | GCGGTTACCGCCCGCGTGT |
| | | CGAACCCAGGTGTGCGAC |
| | | GTCAGACAACGGGGGAGC |
| | | GCTCC |
| SEQ ID NO: 49 | Helper Plasmid (E2A; | TGGTATGGCTTTTTCCCCG |
| | E4orf6; VA RNA (G16A; | TATCCCCCCAGGTGTCTGC |
| | G60A)) | AGGCTCAAAGAGCAGCGA |
| | | GAAGCGTTCAGAGGAAAG |
| | | CGATCCCGTGCCACCTTCC |
| | | CCGTGCCCGGGCTGTCCCC |
| | | GCACGCTGCCGGCTCGGGG |
| | | ATGCGGGGGGAGCGCCGG |
| | | ACCGGAGCGGAGCCCCGG |
| | | GCGGCTCGCTGCTGCCCCC |
| | | TAGCGGGGGAGGGACGTA |
| | | ATTACATCCCTGGGGGCTT |
| | | TGGGGGGGGGCTGTCCCTG |
| | | ATATCTATAACAAGAAAAT |
| | | ATATATATAATAAGTTATC |
| | | ACGTAAGTAGAACATGAA |
| | | ATAACAATATAATTATCGT |
| | | ATGAGTTAAATCTTAAAAG |
| | | TCACGTAAAAGATAATCAT |
| | | GCGTCATTTTGACTCACGC |
| | | GGTCGTTATAGTTCAAAAT |
| | | CAGTGACACTTACCGCATT |
| | | GACAAGCACGCCTCACGG |
| | | GAGCTCCAAGCGGCGACT |
| | | GAGATGTCCTAAATGCACA |
| | | GCGACGGATTCGCGCTATT |
| | | TAGAAAGAGAGAGCAATA |
| | | TTTCAAGAATGCATGCGTC |
| | | AATTTTACGCAGACTATCT |
| | | TTCTAGGGTTAATCTAGCT |
| | | GCATCAGGATCATATCGTC |
| | | GGGTCTTTTTTCCGGCTCA |
| | | GTCATCGCCCAAGCTGGCG |
| | | CTATCTGGGCATCGGGGAG |
| | | GAAGAAGCCCGTGCCTTTT |
| | | CCCGCGAGGTTGAAGCGG |
| | | CATGGAAAGAGTTTGCCGA |
| | | GGATGACTGCTGCTGCATT |
| | | GACGTTGAGCGAAAACGC |
| | | ACGTTTACCATGATGATTC |
| | | GGGAAGGTGTGGCCATGC |
| | | ACGCCTTTAACGGTGAACT |
| | | GTTCGTTCAGGCCACCTGG |
| | | GATACCAGTTCGTCGCGGC |
| | | TTTTCCGGACACAGTTCCG |
| | | GATGGTCAGCCCGAAGCG |
| | | CATCAGCAACCCGAACAAT |
| | | ACCGGCGACAGCCGGAAC |
| | | TGCCGTGCCGGTGTGCAGA |
| | | TTAATGACAGCGGTGCGGC |
| | | GCTGGGATATTACGTCAGC |
| | | GAGGACGGGTATCCTGGCT |
| | | GGATGCCGCAGAAATGGA |
| | | CATGGATACCCCGTGAGTT |
| | | ACCCGGCGGGCGCGCTTGG |
| | | CGTAATCATGGTCATAGCT |
| | | GTTTCCTGTGTGAAATTGT |
| | | TATCCGCTCACAATTCCAC |
| | | ACAACATACGAGCCGGAA |
| | | GCATAAAGTGTAAAGCCTG |
| | | GGGTGCCTAATGAGTGAGC |
| | | TAACTCACATTAATTGCGT |
| | | TGCGCTCACTGCCCGCTTT |
| | | CCAGTCGGGAAACCTGTCG |
| | | TGCCAGCTGCATTAATGAA |
| | | TCGGCCAACGCGCGGGGA |
| | | GAGGCGGTTTGCGTATTGG |
| | | GCGCTCTTCCGCTTCCTCG |
| | | CTCACTGACTCGCTGCGCT |
| | | CGGTCGTTCGGCTGCGGCG |
| | | AGCGGTATCAGCTCACTCA |
| | | AAGGCGGTAATACGGTTAT |
| | | CCACAGAATCAGGGGATA |
| | | ACGCAGGAAAGAACATGT |
| | | GAGCAAAAGGCCAGCAAA |
| | | AGGCCAGGAACCGTAAAA |
| | | AGGCCGCGTTGCTGGCGTT |
| | | TTTCCATAGGCTCCGCCCC |
| | | CCTGACGAGCATCACAAA |
| | | AATCGACGCTCAAGTCAGA |
| | | GGTGGCGAAACCCGACAG |
| | | GACTATAAAGATACCAGG |
| | | CGTTTCCCCCTGGAAGCTC |
| | | CCTCGTGCGCTCTCCTGTT |
| | | CCGACCCTGCCGCTTACCG |
| | | GATACCTGTCCGCCTTTCT |
| | | CCCTTCGGGAAGCGTGGCG |
| | | CTTTCTCATAGCTCACGCT |
| | | GTAGGTATCTCAGTTCGGT |
| | | GTAGGTCGTTCGCTCCAAG |
| | | CTGGGCTGTGTGCACGAAC |
| | | CCCCCGTTCAGCCCGACCG |
| | | CTGCGCCTTATCCGGTAAC |
| | | TATCGTCTTGAGTCCAACC |
| | | CGGTAAGACACGACTTATC |
| | | GCCACTGGCAGCAGCCACT |
| | | GGTAACAGGATTAGCAGA |
| | | GCGAGGTATGTAGGCGGT |
| | | GCTACAGAGTTCTTGAAGT |
| | | GGTGGCCTAACTACGGCTA |
| | | CACTAGAAGGACAGTATTT |
| | | GGTATCTGCGCTCTGCTGA |
| | | AGCCAGTTACCTTCGGAAA |
| | | AAGAGTTGGTAGCTCTTGA |
| | | TCCGGCAAACAAACCACC |
| | | GCTGGTAGCGGTGGTTTTT |
| | | TTGTTTGCAAGCAGCAGAT |
| | | TACGCGCAGAAAAAAAGG |
| | | ATCTCAAGAAGATCCTTTG |
| | | ATCTTTTCTACGGGGTCTG |
| | | ACGCTCAGTGGAACGAAA |
| | | ACTCACGTTAAGGGATTTT |
| | | GGTCATGAGATTATCAAAA |
| | | AGGATCTTCACCTAGATCC |
| | | ITTTAAATTAAAAATGAAG |
| | | TTTTAAATCAATCTAAAGT |
| | | ATATATGAGTAAACTTGGT |
| | | CTGACAGTTACCAATGCTT |
| | | AATCAGTGAGGCACCTATC |
| | | TCAGCGATCTGTCTATTTC |
| | | GTTCATCCATAGTTGCCTG |
| | | ACTCCCCGTCGTGTAGATA |
| | | ACTACGATACGGGAGGGC |
| | | TTACCATCTGGCCCCAGTG |
| | | CTGCAATGATACCGCGAGA |
| | | CCCACGCTCACCGGCTCCA |
| | | GATTTATCAGCAATAAACC |
| | | AGCCAGCCGGAAGGGCCG |
| | | AGCGCAGAAGTGGTCCTGC |
| | | AACTTTATCCGCCTCCATC |
| | | CAGTCTATTAATTGTTGCC |
| | | GGGAAGCTAGAGTAAGTA |
| | | GTTCGCCAGTTAATAGTTT |
| | | GCGCAACGTTGTTGCCATT |
| | | GCTACAGGCATCGTGGTGT |
| | | CACGCTCGTCGTTTGGTAT |
| | | GGCTTCATTCAGCTCCGGT |
| | | TCCCAACGATCAAGGCGA |
| | | GTTACATGATCCCCCATGT |
| | | TGTGCAAAAAAGCGGTTA |
| | | GCTCCTTCGGTCCTCCGAT |
| | | CGTTGTCAGAAGTAAGTTG |
| | | GCCGCAGTGTTATCACTCA |
| | | TGGTTATGGCAGCACTGCA |
| | | TAATTCTCTTACTGTCATG |
| | | CCATCCGTAAGATGCTTTT |
| | | CTGTGACTGGTGAGTACTC |
| | | AACCAAGTCATTCTGAGAA |
| | | TAGTGTATGCGGCGACCGA |
| | | GTTGCTCTTGCCCGGCGTC |
| | | AATACGGGATAATACCGC |
| | | GCCACATAGCAGAACTTTA |
| | | AAAGTGCTCATCATTGGAA |
| | | AACGTTCTTCGGGGCGAAA |
| | | ACTCTCAAGGATCTTACCG |
| | | CTGTTGAGATCCAGTTCGA |
| | | TGTAACCCACTCGTGCACC |
| | | CAACTGATCTTCAGCATCT |
| | | TTTACTTTCACCAGCGTTTC |
| | | TGGGTGAGCAAAAACAGG |
| | | AAGGCAAAATGCCGCAAA |
| | | AAAGGGAATAAGGGCGAC |
| | | ACGGAAATGTTGAATACTC |
| | | ATACTCTTCCTTTTTCAATA |
| | | TTATTGAAGCATTTATCAG |
| | | GGTTATTGTCTCATGAGCG |
| | | GATACATATTTGAATGTAT |
| | | TTAGAAAAATAAACAAAT |
| | | AGGGGTTCCGCGCACATTT |
| | | CCCCGAAAAGTGCCACCTA |
| | | AATTGTAAGCGTTAATATT |
| | | TTGTTAAAATTCGCGTTAA |
| | | ATTTTTGTTAAATCAGCTC |
| | | ATTTTTTAACCAATAGGCC |
| | | GAAATCGGCAAAATCCCTT |
| | | ATAAATCAAAAGAATAGA |
| | | CCGAGATAGGGTTGAGTGT |
| | | TGTTCCAGTTTGGAACAAG |
| | | AGTCCACTATTAAAGAACG |
| | | TGGACTCCAACGTCAAAGG |
| | | GCGAAAAACCGTCTATCAG |
| | | GGCGATGGCCCACTACGTG |
| | | AACCATCACCCTAATCAAG |
| | | TTTTTTGGGGTCGAGGTGC |
| | | CGTAAAGCACTAAATCGG |
| | | AACCCTAAAGGGAGCCCC |
| | | CGATTTAGAGCTTGACGGG |
| | | GAAAGCCGGCGAACGTGG |
| | | CGAGAAAGGAAGGGAAGA |
| | | AAGCGAAAGGAGCGGGCG |
| | | CTAGGGCGCTGGCAAGTGT |
| | | AGCGGTCACGCTGCGCGTA |
| | | ACCACCACACCCGCCGCGC |
| | | TTAATGCGCCGCTACAGGG |
| | | CGCGTCCCATTCGCCATTC |
| | | AGGCTGCGCAACTGTTGGG |
| | | AAGGGCGATCGGTGCGGG |
| | | CCTCTTCGCTATTACGCCA |
| | | GCTGGCGAAAGGGGGATG |
| | | TGCTGCAAGGCGATTAAGT |
| | | TGGGTAACGCCAGGGTTTT |
| | | CCCAGTCACGACGTTGTAA |
| | | AACGACGGCCAGTGAGCG |
| | | CGCCTCGTTCATTCACGTT |
| | | TTTGAACCCGTGGAGGACG |
| | | GGCAGACTCGCGGTGCAA |
| | | ATGTGTTTTACAGCGTGAT |
| | | GGAGCAGATGAAGATGCT |
| | | CGACACGCTGCAGAACAC |
| | | GCAGCTAGATTAACCCTAG |
| | | AAAGATAATCATATTGTGA |
| | | CGTACGTTAAAGATAATCA |
| | | TGTGTAAAATTGACGCATG |
| | | TGTTTTATCGGTCTGTATAT |
| | | CGAGGTTTATTTATTAATT |
| | | TGAATAGATATTAAGTTTT |
| | | ATTATATTTACACTTACAT |
| | | ACTAATAATAAATTCAACA |
| | | AACAATTTATTTATGTTTA |
| | | TTTATTTATTAAAAAAAAC |
| | | AAAAACTCAAAATTTCTTC |
| | | TATAAAGTAACAAAACTTT |
| | | TATGAGGGACAGCCCCCCC |
| | | CCAAAGCCCCCAGGGATGT |
| | | AATTACGTCCCTCCCCCGC |
| | | TAGGGGGCAGCAGCGAGC |
| | | CGCCCGGGGCTCCGCTCCG |
| | | GTCCGGCGCTCCCCCCGCA |
| | | TCCCCGAGCCGGCAGCGTG |
| | | CGGGGACAGCCCGGGCAC |
| | | GGGGAAGGTGGCACGGGA |
| | | TCGCTTTCCTCTGAACGCT |
| | | TCTCGCTGCTCTTTGAGCC |
| | | TGCAGACACCTGGGGGGA |
| | | TACGGGGAAAAGGCCTCC |
| | | ACGGCCACTAGTCCATAGA |
| | | GCCCACCGCATCCCCAGCA |
| | | TGCCTGCTATTGTCTTCCC |
| | | AATCCTCCCCCTTGCTGTC |
| | | CTGCCCCACCCCACCCCCT |
| | | AGAATAGAATGACACCTA |
| | | CTCAGACAATGCGATGCAA |
| | | TTTCCTCATTTTATTAGGA |
| | | AAGGACAGTGGGAGTGGC |
| | | ACCTTCCAGGGTCAAGGAA |
| | | GGCACGGGGGAGGGGCAA |
| | | ACAACAGATGGCTGGCAA |
| | | CTAGAAGGCACAGCTACAT |
| | | GGGGGTAGAGTCATAATC |
| | | GTGCATCAGGATAGGGCG |
| | | GTGGTGCTGCAGCAGCGCG |
| | | CGAATAAACTGCTGCCGCC |
| | | GCCGCTCCGTCCTGCAGGA |
| | | ATACAACATGGCAGTGGTC |
| | | TCCTCAGCGATGATTCGCA |
| | | CCGCCCGCAGCATGAGAC |
| | | GCCTTGTCCTCCGGGCACA |
| | | GCAGCGCACCCTGATCTCA |
| | | CTTAAATCAGCACAGTAAC |
| | | TGCAGCACAGCACCACAAT |
| | | ATTGTTCAAAATCCCACAG |
| | | TGCAAGGCGCTGTATCCAA |
| | | AGCTCATGGCGGGGACCA |
| | | CAGAACCCACGTGGCCATC |
| | | ATACCACAAGCGCAGGTA |
| | | GATTAAGTGGCGACCCCTC |
| | | ATAAACACGCTGGACATA |
| | | AACATTACCTCTTTTGGCA |
| | | TGTTGTAATTCACCACCTC |
| | | CCGGTACCATATAAACCTC |
| | | TGATTAAACATGGCGCCAT |
| | | CCACCACCATCCTAAACCA |
| | | GCTGGCCAAAACCTGCCCG |
| | | CCGGCTATGCACTGCAGGG |
| | | AACCGGGACTGGAACAAT |
| | | GACAGTGGAGAGCCCAGG |
| | | ACTCGTAACCATGGATCAT |
| | | CATGCTCGTCATGATATCA |
| | | ATGTTGGCACAACACAGGC |
| | | ACACGTGCATACACTTCCT |
| | | CAGGATTACAAGCTCCTCC |
| | | CGCGTCAGAACCATATCCC |
| | | AGGGAACAACCCATTCCTG |
| | | AATCAGCGTAAATCCCACA |
| | | CTGCAGGGAAGACCTCGC |
| | | ACGTAACTCACGTTGTGCA |
| | | TTGTCAAAGTGTTACATTC |
| | | GGGCAGCAGCGGATGATC |
| | | CTCCAGTATGGTAGCGCGG |
| | | GTCTCTGTCTCAAAAGGAG |
| | | GTAGGCGATCCCTACTGTA |
| | | CGGAGTGCGCCGAGACAA |
| | | CCGAGATCGTGTTGGTCGT |
| | | AGTGTCATGCCAAATGGAA |
| | | CGCCGGACGTAGTCATGGT |
| | | TGTGGCCATATTATCATCG |
| | | TGTTTTTCAAAGGAAAACC |
| | | ACGTCCCCGTGGTTCGGGG |
| | | GGCCTAGACGTTTTTTTAA |
| | | CCTCGACTAAACACATGTA |
| | | AAGCATGTGCACCGAGGC |
| | | CCCAGATCAGATCCCATAC |
| | | AATGGGGTACCTTCTGGGC |
| | | ATCCTTCAGCCCCTTGTTG |
| | | AATACGCTTGAGGAGAGC |
| | | CATTTGACTCTTTCCACAA |
| | | CTATCCAACTCACAACGTG |
| | | GCACTGGGGTTGTGCCGCC |
| | | TTTGCAGGTGTATCTTATA |
| | | CACGTGGCTTTTGGCCGCA |
| | | GAGGCACCTGTCGCCAGGT |
| | | GGGGGGTTCCGCTGCCTGC |
| | | AAAGGGTCGCTACAGACG |
| | | TTGTTTGTCTTCAAGAAGC |
| | | TTCCAGAGGAACTGCTTCC |
| | | TTCACGACATTCAACAGAC |
| | | CTTGCATTCCTTTGGCGAG |
| | | AGGGGAAAGACCCCTAGG |
| | | AATGCTCGTCAAGAAGAC |
| | | AGGGCCAGGTTTCCGGGCC |
| | | CTCACATTGCCAAAAGACG |
| | | GCAATATGGTGGAAAATA |
| | | ACATATAGACAAACGCAC |
| | | ACCGGCCTTATTCCAAGCG |
| | | GCTTCGGCCAGTAACGTTA |
| | | GGGGGGGGGGAGGGAGAG |
| | | GGGCTTAAAAATCAAAGG |
| | | GGTTCTGCCGCGCATCACT |
| | | ATGCGCCACTGGCAGGGA |
| | | CACGTTGCGATACTGGTGT |
| | | TTAGTGCTCCACTTAAACT |
| | | CAGGCACAACCATCCGCG |
| | | GCAGCTCGGTGAAGTTTTC |
| | | ACTCCACAGGCTGCGCACC |
| | | ATCACCAACGCGTTTAGCA |
| | | GGTCGGGCGCCGATATCTT |
| | | GAAGTCGCAGTTGGGGCCT |
| | | CCGCCCTGCGCGCGCGAGT |
| | | TGCGATACACAGGGTTGCA |
| | | GCACTGGAACACTATCAGC |
| | | GCCGGGTGGTGCACGCTGG |
| | | CCAGCACGCTCTTGTCGGA |
| | | GATCAGATCCGCGTCCAGG |
| | | TCCTCCGCGTTGCTCAGGG |
| | | CGAACGGAGTCAACTTTGG |
| | | TAGCTGCCTTCCCAAAAAG |
| | | GGTGCATGCCCAGGCTTTG |
| | | AGTTGCACTCGCACCGTAG |
| | | TGGCATCAGAAGGTGACC |
| | | GTGCCCGGTCTGGGCGTTA |
| | | GGATACAGCGCCTGCATGA |
| | | AAGCCTTGATCTGCTTAAA |
| | | AGCCACCTGAGCCTTTGCG |
| | | CCTTCAGAGAAGAACATGC |
| | | CGCAAGACTTGCCGGAAA |
| | | ACTGATTGGCCGGACAGGC |
| | | CGCGTCATGCACGCAGCAC |
| | | CTTGCGTCGGTGTTGGAGA |
| | | TCTGCACCACATTTCGGCC |
| | | CCACCGGTTCTTCACGATC |
| | | TTGGCCTTGCTAGACTGCT |
| | | CCTTCAGCGCGCGCTGCCC |
| | | GTTTTCGCTCGTCACATCC |
| | | ATTTCAATCACGTGCTCCT |
| | | TATTTATCATAATGCTCCC |
| | | GTGTAGACACTTAAGCTCG |
| | | CCTTCGATCTCAGCGCAGC |
| | | GGTGCAGCCACAACGCGC |
| | | AGCCCGTGGGCTCGTGGTG |
| | | CTTGTAGGTTACCTCTGCA |
| | | AACGACTGCAGGTACGCCT |
| | | GCAGGAATCGCCCCATCAT |
| | | CGTCACAAAGGTCTTGTTG |
| | | CTGGTGAAGGTCAGCTGCA |
| | | ACCCGCGGTGCTCCTCGTT |
| | | TAGCCAGGTCTTGCATACG |
| | | GCCGCCAGAGCTTCCACTT |
| | | GGTCAGGCAGTAGCTTGAA |
| | | GTTTGCCTTTAGATCGTTA |
| | | TCCACGTGGTACTTGTCCA |
| | | TCAACGCGCGCGCAGCCTC |
| | | CATGCCCTTCTCCCACGCA |
| | | GACACGATCGGCAGGCTC |
| | | AGCGGGTTTATCACCGTGC |
| | | TTTCACTTTCCGCTTCACTG |
| | | GACTCTTCCTTTTCCTCTTG |
| | | CGTCCGCATACCCCGCGCC |
| | | ACTGGGTCGTCTTCATTCA |
| | | GCCGCCGCACCGTGCGCTT |
| | | ACCTCCCTTGCCGTGCTTG |
| | | ATTAGCACCGGTGGGTTGC |
| | | TGAAACCCACCATTTGTAG |
| | | CGCCACATCTTCTCTTTCTT |
| | | CCTCGCTGTCCACGATCAC |
| | | CTCTGGGGATGGCGGGCGC |
| | | TCGGGCTTGGGAGAGGGG |
| | | CGCTTCTTTTTCTTTTTGGA |
| | | CGCAATGGCCAAATCCGCC |
| | | GTCGAGGTCGATGGCCGCG |
| | | GGCTGGGTGTGCGCGGCAC |
| | | CAGCGCATCTTGTGACGAG |
| | | TCTTCTTCGTCCTCGGACTC |
| | | GAGACGCCGCCTCAGCCGC |
| | | TTTTTTGGGGGCGCGCGCT |
| | | TGTCGTCATCGTCTTTGTA |
| | | GTCGGGAGGCGGCGGCGA |
| | | CGGCGACGGGGACGACAC |
| | | GTCCTCCATGGTTGGTGGA |
| | | CGTCGCGCCGCACCGCGTC |
| | | CGCGCTCGGGGGTGGTTTC |
| | | GCGCTGCTCCTCTTCCCGA |
| | | CTGGCCATGGTGGCCGAGG |
| | | ATAACTTCGTATATGGTTT |
| | | CTTATACGAAGTTATGATC |
| | | CAGACATGATAAGATACAT |
| | | TGATGAGTTTGGACAAACC |
| | | ACAACTAGAATGCAGTGA |
| | | AAAAAATGCTTTATTTGTG |
| | | AAATTTGTGATGCTATTGC |
| | | TTTATTTGTAACCATTATA |
| | | AGCTGCAATAAACAAGTTA |
| | | ACAACAACAATTGCATTCA |
| | | TTTTATGTTTCAGGTTCAG |
| | | GGGGAGGTGTGGGAGGTT |
| | | TTTTAAAGCAAGTAAAACC |
| | | TCTACAAATGTGGTATGGC |
| | | TGATTATGATCCTCTAGAG |
| | | TCGCAGATCTGCTACGTAT |
| | | CAAGCTGTGGCAGGGAAA |
| | | CCCTCTGCCTCCCCCGTGA |
| | | TGTAATACTTTTGCAAGGA |
| | | ATGCGATGAAGTAGAGCC |
| | | CGCAGTGGCCAAGTGGCTT |
| | | TGGTCCGTCTCCTCCACGG |
| | | ATGCCCCTCCACGGCTAGT |
| | | GGGCGCATGTAGGCGGTG |
| | | GGCGTCCGCCGCCTCCAGC |
| | | AGCAGGTCATAGAGGGGC |
| | | ACCACGTTCTTGCACTTCA |
| | | TGCTGTACAGATGCTCCAT |
| | | GCCTTTGTTACTCATGTGT |
| | | CGGATGTGGGAGAGGATG |
| | | AGGAGGAGCTGGGCCAGC |
| | | CGCTGGTGCTGCTGCTGCA |
| | | GGGTCAGGCCTGCCTTGGC |
| | | CATCAGGTGGATCAAAGTG |
| | | TCTGTGATCTTGTCCAGGA |
| | | CTCGGTGGATATGGTCCTT |
| | | CTCTTCCAGAGACTTCAGG |
| | | GTGCTGGACAGAAATGTGT |
| | | ACACTCCAGAATTAAGCAA |
| | | AATAATAGATTTGAGGCAC |
| | | ACAAACTCCTCTCCCTGCA |
| | | GATTCATCATGCGGAACCG |
| | | AGATGATGTAGCCAGCAG |
| | | CATGTCGAAGATCTCCACC |
| | | ATGCCCTCTACACATTTTC |
| | | CCTGGTTCCTGTCCAAGAG |
| | | CAAGTTAGGAGCAAACAG |
| | | TAGCTTCACTGGGTGCTCC |
| | | ATGGAGCGCCAGACGAGA |
| | | CCAATCATCAGGATCTCTA |
| | | GCCAGGCACATTCTAGAAG |
| | | GTGGACCTGATCATGGAGG |
| | | GTCAAATCCACAAAGCCTG |
| | | GCACCCTCTTCGCCCAGTT |
| | | GATCATGTGAACCAGCTCC |
| | | CTGTCTGCCAGGTTGGTCA |
| | | GTAAGCCCATCATCGAAGC |
| | | TTCACTGAAGGGTCTGGTA |
| | | GGATCATACTCGGAATAGA |
| | | GTATGGGGGGCTCAGCATC |
| | | CAACAAGGCACTGACCATC |
| | | TGGTCGGCCGTCAGGGACA |
| | | AGGCCAGGCTGTTCTTCTT |
| | | AGAGCGTTTGATCATGAGC |
| | | GGGCTTGGCCAAAGGTTGG |
| | | CAGCTCTCATGTCTCCAGC |
| | | AGATGGCTCGAGATCGCCA |
| | | TCTTCCAGCAGGCGCACCA |
| | | TTGCCCCTGTTTCACTATCC |
| | | AGGTTACGGATATAGTTCA |
| | | TGACAATATTTACATTGGT |
| | | CCAGCCACCAGCTTGCATG |
| | | ATCTCCGGTATTGAAACTC |
| | | CAGCGCGGGCCATATCTCG |
| | | CGCGGCTCCGACACGGGC |
| | | ACTGTGTCCAGACCAGGCC |
| | | AGGTATCTCTGACCAGAGT |
| | | CATCCTAAAATACACAAAC |
| | | AATTAGAATCAGTAGTTTA |
| | | ACACATTATACACTTAAAA |
| | | ATTTTATATTTACCTTAGC |
| | | GCCGTAAATCAATCGATGA |
| | | GTTGCTTCAAAAATCCCTT |
| | | CCAGGGCGCGAGTTGATA |
| | | GCTGGCTGGTGGCAGATGG |
| | | CGCGGCAACACCATTTTTT |
| | | CTGACCCGGCAAAACAGG |
| | | TAGTTATTCGGATCATCAG |
| | | CTACACCAGAGACGGAAA |
| | | TCCATCGCTCGACCAGTTT |
| | | AGTGACTCCCAGGCTAAGT |
| | | GCCTTCTCTACACCTGCGG |
| | | TGCTAACCAGCGTTTTCGT |
| | | TCTGCCAATATGGATTAAC |
| | | ATTCTCCCACCGTCAGTAC |
| | | GTGAGATATCTTTAACCCT |
| | | GATCCTGGCAATTTCGGCT |
| | | ATACGTAACAGGGTGTTAT |
| | | AAGCAATCCCCAGAAATG |
| | | CCAGATTACGTATATCCTG |
| | | GCAGCGATCGCTATTTTCC |
| | | ATGAGTGAACGGACTTGGT |
| | | CGAAATCAGTGCGTTCGAA |
| | | CGCTAGAGCCTGTTTTGCA |
| | | CGTTCACCGGCATCAACGT |
| | | TTTCTTTTCGGATCCGCCG |
| | | CATAACCAGTGAAACAGC |
| | | ATTGCTGTCACTTGGTCGT |
| | | GGCAGCCCGGACCGACGA |
| | | TGAAGCATGTTTAGCTGGC |
| | | CCAAATGTTGCTGGATAGT |
| | | TTTTACTGCCAGACCGCGC |
| | | GCTTGAAGATATAGAAGAT |
| | | AATCGCGAACATCTTCAGG |
| | | TTCTGCGGGAAACCATTTC |
| | | CGGTTATTCAACTTGCACC |
| | | ATGCCGCCCACGACCGGCA |
| | | AACGGACAGAAGCATTTTC |
| | | CAGGTATGCTCAGAAAAC |
| | | GCCTGGCGATCCCTGAACA |
| | | TGTCCATCAGGTTCTTGCG |
| | | AACCTCATCACTCGTTGCA |
| | | TCGACCGGTAATGCAGGCA |
| | | AATTTTGGTGTACGGTCAG |
| | | TAAATTGGACATGGTGGCT |
| | | ACGTAATAACTTCGTATAT |
| | | GGTTTCTTATACGAAGTTA |
| | | TGCGGCCGCTTTACGAGGG |
| | | TAGGAAGTGGTACGGAAA |
| | | GTTGGTATAAGACAAAAGT |
| | | GTTGTGGAATTGCTCCAGG |
| | | CGATCTGACGGTTCACTAA |
| | | ACGAGCTCTGCTTTTATAG |
| | | GCGCCCACCGTACACGCCT |
| | | AAAGCTTATACGTTCTCTA |
| | | TCACTGATAGGGAGTAAAC |
| | | TGGATATACGTTCTCTATC |
| | | ACTGATAGGGAGTAAACT |
| | | GTAGATACGTTCTCTATCA |
| | | CTGATAGGGAGTAAACTG |
| | | GTCATACGTTCTCTATCAC |
| | | TGATAGGGAGTAAACTCCT |
| | | TATACGTTCTCTATCACTG |
| | | ATAGGGAGTAAAGTCTGC |
| | | ATACGTTCTCTATCACTGA |
| | | TAGGGAGTAAACTCTTCAT |
| | | ACGTTCTCTATCACTGATA |
| | | GGGAGTAAACTCGCGGCC |
| | | GCAGAGAAATGTTCTGGCA |
| | | CCTGCACTTGCACTGGGGA |
| | | CAGCCTATTTTGCTAGTTT |
| | | GTTTTGTTTCGTTTTGTTTT |
| | | GATGGAGAGCGTATGTTAG |
| | | TACTATCGATTCACACAAA |
| | | AAACCAACACACAGATGT |
| | | AATGAAAATAAAGATATTT |
| | | TATTGGATCTGCGATCGCT |
| | | CCGGTGCCCGTCAGTGGGC |
| | | AGAGCGCACATCGCCCAC |
| | | AGTCCCCGAGAAGTTGGG |
| | | GGGAGGGGTCGGCAATTG |
| | | AACGGGTGCCTAGAGAAG |
| | | GTGGCGCGGGGTAAACTG |
| | | GGAAAGTGATGTCGTGTAC |
| | | TGGCTCCGCCTTTTTCCCG |
| | | AGGGTGGGGGAGAACCGT |
| | | ATGTAAGTGCAGTAGTCGC |
| | | CGTGAACGTTCTTTTTCGC |
| | | AACGGGTTTGCCGCCAGAA |
| | | CACAGCTGAAGCTTCGAGG |
| | | GGCTCGCATCTCTCCTTCA |
| | | CGCGCCCGCCGCCCTACCT |
| | | GAGGCCGCCATCCACGCCG |
| | | GTTGAGTCGCGTTCTGCCG |
| | | CCTCCCGCCTGTGGTGCCT |
| | | CCTGAACTGCGTCCGCCGT |
| | | CTAGGTAAGTTTAAAGCTC |
| | | AGGTCGAGACCGGGCCTTT |
| | | GTCCGGCGCTCCCTTGGAG |
| | | CCTACCTAGACTCAGCCGG |
| | | CTCTCCACGCTTTGCCTGA |
| | | CCCTGCTTGCTCAACTCTA |
| | | CGTCTTTGTTTCGTTTTCTG |
| | | TTCTGCGCCGTTACAGATC |
| | | CAAGCTGTGACCGGCGCCT |
| | | ACGCTAGCGGATCCGCCGC |
| | | CACCATGTCTAGACTGGAC |
| | | AAGAGCAAAGTCATAAAC |
| | | TCTGCTCTGGAATTACTCA |
| | | ATGGAGTCGGTATCGAAG |
| | | GCCTGACGACAAGGAAAC |
| | | TCGCTCAAAAGCTGGGAGT |
| | | TGAGCAGCCTACCCTGTAC |
| | | TGGCACGTGAAGAACAAG |
| | | CGGGCCCTGCTCGATGCCC |
| | | TGCCAATCGAGATGCTGGA |
| | | CAGGCATCATACCCACTCC |
| | | TGCCCCCTGGAAGGCGAGT |
| | | CATGGCAAGACTTTCTGCG |
| | | GAACAACGCCAAGTCATA |
| | | CCGCTGTGCTCTTCTCTCA |
| | | CATCGCGACGGGGCTAAA |
| | | GTGCATCTCGGCACCCGCC |
| | | CAACAGAGAAACAGTACG |
| | | AAACCCTGGAAAATCAGCT |
| | | CGCGTTCCTGTGTCAGCAA |
| | | GGCTTCTCCCTGGAGAACG |
| | | CACTGTACGCTCTGTCCGC |
| | | CGTGGGCCACTTTACACTG |
| | | GGCTGCGTATTGGAGGAAC |
| | | AGGAGCATCAAGTAGCAA |
| | | AAGAGGAAAGAGAGACAC |
| | | CTACCACCGATTCTATGCC |
| | | CCCACTTCTGAAACAAGCA |
| | | ATTGAGCTGTTCGACCGGC |
| | | AGGGAGCCGAACCTGCCTT |
| | | CCTTTTCGGCCTGGAACTA |
| | | ATCATATGTGGCCTGGAGA |
| | | AACAGCTAAAGTGCGAAA |
| | | GCGGCGGGCCGACCGACG |
| | | CCCTTGACGATTTTGACTT |
| | | AGACATGCTCCCAGCCGAT |
| | | GCCCTTGACGACTTTGACC |
| | | TTGATATGCTGCCTGCTGA |
| | | CGCTCTTGACGATTTTGAC |
| | | CTTGACATGCTCCCCGGGT |
| | | GAACCGGTCGCTGATCAGC |
| | | CTCGACTGTGCCTTCTAGT |
| | | TGCCAGCCATCTGTTGTTT |
| | | GCCCCTCCCCCGTGCCTTC |
| | | CTTGACCCTGGAAGGTGCC |
| | | ACTCCCACTGTCCTTTCCT |
| | | AATAAAATGAGGAAATTG |
| | | CATCGCATTGTCTGAGTAG |
| | | GTGTCATTCTATTCTGGGG |
| | | GGTGGGGTGGGGCAGGAC |
| | | AGCAAGGGGGAGGATTGG |
| | | GAAGACAATAGCAGGCAT |
| | | GCTGGGGATGCGGTGGGCT |
| | | CTATGGCTTCTGAGGCGGA |
| | | AAGAACCAGCTGGGGCTC |
| | | GACTAGAGCTTGCGGAACC |
| | | CTTAGAGGGCCTATTTCCC |
| | | ATGATTCCTTCATATTTGC |
| | | ATATACGATACAAGGCTGT |
| | | TAGAGAGATAATTAGAATT |
| | | AATTTGACTGTAAACACAA |
| | | AGATATTAGTACAAAATAA |
| | | TAACTTCGTATAATGTATG |
| | | CTATACGAAGTTATCAGAC |
| | | ATGATAAGATACATTGATG |
| | | AGTTTGGACAAACCACAAC |
| | | TAGAATGCAGTGAAAAAA |
| | | ATGCTTTATTTGTGAAATT |
| | | TGTGATGCTATTGCTTTATT |
| | | TGTAACCATTATAAGCTGC |
| | | AATAAACAAGGTACCTCA |
| | | AGCGCCGGGTTTTCGCGTC |
| | | ATGCACCACGTCCGTGGTT |
| | | CAAGCGCCGGGTTTTCGCG |
| | | TCATGCACCACGTCCGTGG |
| | | GCCCTCGGGTACTTCAACG |
| | | TCAGCAGTAACTGTAAATC |
| | | CGAGCCGTTCATAGAAGG |
| | | GCAAATTCCTTGGCGCTGA |
| | | CGTTTCAAGAAAGGCTGGC |
| | | ACTCCGGCTCGTTCTGCGG |
| | | CTTCTACTCCGGGCAATAC |
| | | CACCGCGGAACCAAGGCC |
| | | CTTTCCCTGATGATCGGGG |
| | | CTAACGCCCACAGTAGCGA |
| | | GGAACCAAGCTGGTTCTTT |
| | | AGGGCGGTGAGGGGCGAG |
| | | GAGTCCTTCCATTTGTTGC |
| | | TGAGCCGCGAGACGAGAG |
| | | CCACTAAGCTCAGCCATTC |
| | | GGGGACCAATTTCTGCAAA |
| | | TACAGCCCCGGCCTCAACG |
| | | CTCTCCGGAGTCGTCCACA |
| | | CTGCCACTGCAGCCCCGTC |
| | | GTCGGCGACCCAAACTTTA |
| | | CCGATGTCCAATCCTACCC |
| | | TGGTCAAAAAAAGTTCTTG |
| | | CAATTCTGTAACCCGTTCA |
| | | ATATGTCTATCAGGATCAA |
| | | CTGTGTGGCGTGTAGCGGG |
| | | ATAATCCGCGAAAGCGGC |
| | | AGCCAATGTTCTCACGGCC |
| | | CTAGGGACGTCGTCTCGAG |
| | | TTGCCAGTCTGACAGTAGG |
| | | TTTATATTCTGTCATGGTG |
| | | GCGGCGAATTCTCTTCTAT |
| | | GGAGGTCAAAACAGCGTG |
| | | GATGGCGTCTCCAGGCGAT |
| | | CTGACGGTTCACTAAACGA |
| | | GCTCTGCTTATATAAACCT |
| | | CCCACCGTACACGCCTACC |
| | | GCCCATTTGCGTCAATGGG |
| | | GCGGAGTTGTTACGACATT |
| | | TTGGAAAGTCCCGTTGATT |
| | | TTGGTGCCAAAACAAACTC |
| | | CCATTGACGTCAATGGGGT |
| | | GGAGACTTGGAAATCCCCG |
| | | TGAGTCAAACCGCTATCCA |
| | | CGCCCATTGATGTACTGCC |
| | | AAAACCGCATCACCATGGT |
| | | AATAGCGATGACTAATACG |
| | | TAGATGTACTGCCAAGTAG |
| | | GAAAGTCCCATAAGGTCAT |
| | | GTACTGGGCATAATACTAG |
| | | TTCTTGGGAAAAGCGCTCC |
| | | CCTACCCATAACTTCGTAT |
| | | AATGTATGCTATACGAAGT |
| | | TATTTTGCAGTTTTAAAAT |
| | | TATGTTTTAAAATGGACTA |
| | | TCATATGCTTACCGTAACT |
| | | TGAAAGTATTTCGATTTCT |
| | | TGGCTTTATATATCTTGTG |
| | | GAAAGGACGAAACACCGG |
| | | GCACTCTTCCGTGATCTGG |
| | | TGGATAAATTCGCAAGGGT |
| | | ATCATGGCGGACGACCGG |
| | | GATTCGAACCCCGGATCCG |
| | | GCCGTCCGCCGTGATCCAT |
| | | GCGGTTACCGCCCGCGTGT |
| | | CGAACCCAGGTGTGCGAC |
| | | GTCAGACAACGGGGGAGC |
| | | GCTCCTTTTTGGGCCCAT |
| SEQ ID NO: 50 | N-terminal Blasticidin | GAAAACATTTAACATTT |
| | fragment/N-terminal | CTCAACAGGATCTAGAA |
| | NpuDnaE Intein | TTAGTAGAAGTAGCGAC |
| | | AGAGAAGATTACAATGC |
| | | TTTATGAGGATAATAAA |
| | | CATCATGTGGGAGCGGC |
| | | AATTCGTACGAAAACAG |
| | | GAGAAATCATTTCGGCA |
| | | GTACATATTGAAGCGTA |
| | | TATAGGACGAGTAACTG |
| | | TTTGTGCAGAAGCCATT |
| | | GCGATTGGTAGTGCAGT |
| | | TTCGAATGGACAAAAGG |
| | | ATTTTGACACGATTGTA |
| | | GCTGTTAGACACCCTTA |
| | | TTCTGACGAAGTAGATA |
| | | GAAGTATTCGAGTGGTA |
| | | AGTCCTTGTGGTATGTG |
| | | CCTTTCATACGAGACCG |
| | | AGATCCTGACTGTCGAG |
| | | TACGGATTGCTTCCTATC |
| | | GGCAAAATCGTGGAGAA |
| | | GAGGATTGAATGTACCG |
| | | TCTATTCAGTCGATAAT |
| | | AATGGGAACATCTACAC |
| | | ACAGCCCGTGGCTCAAT |
| | | GGCACGACAGAGGAGA |
| | | GCAGGAAGTTTTTGAAT |
| | | ACTGTCTCGAGGACGGA |
| | | TCCCTCATCCGCGCTACT |
| | | AAAGATCATAAGTTTAT |
| | | GACCGTGGACGGCCAGA |
| | | TGCTGCCAATTGACGAA |
| | | ATTTTTGAACGAGAGCT |
| | | GGATCTGATGAGAGTCG |
| | | ACAACCTTCCAAACTGA |
| SEQ ID NO: 51 | C-terminal NpuDnaE | ATGATTAAGATCGCTAC |
| | Intein/C-terminal | GCGGAAGTACCTGGGGA |
| | Blasticidin | AACAGAACGTCTACGAC |
| | fragment | ATAGGTGTGGAGCGCGA |
| | | TCACAACTTTGCTCTGA |
| | | AAAATGGATTTATCGCC |
| | | AGCAACTGTAGGGAGTT |
| | | GATTTCAGACTATGCAC |
| | | CAGATTGTTTTGTGTTAA |
| | | TAGAAATGAATGGCAAG |
| | | TTAGTCAAAACTACGAT |
| | | TGAAGAACTCATTCCAC |
| | | TCAAATATACCCGAAAT |
| | | T |
| SEQ ID NO: 52 | GFP AAV (ITR to ITR) | GCTTTTGGCCACTCCCTC |
| | | TCTGCGCGCTCGCTCGCT |
| | | CACTGAGGCCGGGCGAC |
| | | CAAAGGTCGCCCGACGC |
| | | CCGGGCTTTGCCCGGGC |
| | | GGCCTCAGTGAGCGAGC |
| | | GAGCGCGCAGAGAGGG |
| | | AGTGGCCAACTCCATCA |
| | | CTAGGGGTTCCTCTGCA |
| | | GCCGCGACCGGCCAAGG |
| | | TTTAATGATAGGCTGCA |
| | | ACGGGATGTTGGGAATA |
| | | TGTTGCACTGGTCCGTG |
| | | AGGGTACCAACTTGTTT |
| | | ATTGCAGCTTATAATGG |
| | | TTACAAATAAAGCAATA |
| | | GCATCACAAATTTCACA |
| | | AATAAAGCATTTTTTTCA |
| | | CTGCATTCTAGTTGTGGT |
| | | TTGTCCAAACTCATCAA |
| | | TGTATCTTATCATGTCTG |
| | | ACCGGTTCACTTGAGCT |
| | | CGAGATCTGAGTACTTG |
| | | TACAGCTCGTCCATGCC |
| | | GAGAGTGATCCCGGCGG |
| | | CGGTCACGAACTCCAGC |
| | | AGGACCATGTGATCGCG |
| | | CTTCTCGTTGGGGTCTTT |
| | | GCTCAGGGCGGACTGGG |
| | | TGCTCAGGTAGTGGTTG |
| | | TCGGGCAGCAGCACGGG |
| | | GCCGTCGCCGATGGGGG |
| | | TGTTCTGCTGGTAGTGGT |
| | | CGGCGAGCTGCACGCTG |
| | | CCGTCCTCGATGTTGTG |
| | | GCGGATCTTGAAGTTCA |
| | | CCTTGATGCCGTTCTTCT |
| | | GCTTGTCGGCCATGATA |
| | | TAGACGTTGTGGCTGTT |
| | | GTAGTTGTACTCCAGCTT |
| | | GTGCCCCAGGATGTTGC |
| | | CGTCCTCCTTGAAGTCG |
| | | ATGCCCTTCAGCTCGAT |
| | | GCGGTTCACCAGGGTGT |
| | | CGCCCTCGAACTTCACC |
| | | TCGGCGCGGGTCTTGTA |
| | | GTTGCCGTCGTCCTTGA |
| | | AGAAGATGGTGCGCTCC |
| | | TGGACGTAGCCTTCGGG |
| | | CATGGCGGACTTGAAGA |
| | | AGTCGTGCTGCTTCATGT |
| | | GGTCGGGGTAGCGGCTG |
| | | AAGCACTGCACGCCGTA |
| | | GGTCAGGGTGGTCACGA |
| | | GGGTGGGCCAGGGCACG |
| | | GGCAGCTTGCCGGTGGT |
| | | GCAGATGAACTTCAGGG |
| | | TCAGCTTGCCGTAGGTG |
| | | GCATCGCCCTCGCCCTC |
| | | GCCGGACACGCTGAACT |
| | | TGTGGCCGTTTACGTCG |
| | | CCGTCCAGCTCGACCAG |
| | | GATGGGCACCACCCCGG |
| | | TGAACAGCTCCTCGCCC |
| | | TTGCTCACCATGGTGGC |
| | | GGCTTAAGGGTTCGATC |
| | | CTCTAGAGTCCGGAGGC |
| | | TGGATCGGTCCCGGTGT |
| | | CTACTATGGAGGTCAAA |
| | | ACAGCGTGGATGGCGTC |
| | | TCCAGGCGATCTGACGG |
| | | TTCACTAAACGAGCTCT |
| | | GCTTATATAGACCTCCC |
| | | ACCGTACACGCCTACCG |
| | | CCCATTTGCGTCAATGG |
| | | GGCGGAGTTGTTACGAC |
| | | ATTTTGGAAAGTCCCGT |
| | | TGATTTTGGTGCCAAAA |
| | | CAAACTCCCATTGACGT |
| | | CAATGGGGTGGAGACTT |
| | | GGAAATCCCCGTGAGTC |
| | | AAACCGCTATCCACGCC |
| | | CATTGATGTACTGCCAA |
| | | AACCGCATCACCATGGT |
| | | AATAGCGATGACTAATA |
| | | CGTAGATGTACTGCCAA |
| | | GTAGGAAAGTCCCATAA |
| | | GGTCATGTACTGGGCAT |
| | | AATGCCAGGCGGGCCAT |
| | | TTACCGTCATTGACGTC |
| | | AATAGGGGGCGTACTTG |
| | | GCATATGATACACTTGA |
| | | TGTACTGCCAAGTGGGC |
| | | AGTTTACCGTAAATACT |
| | | CCACCCATTGACGTCAA |
| | | TGGAAAGTCCCTATTGG |
| | | CGTTACTATGGGAACAT |
| | | ACGTCATTATTGACGTC |
| | | AATGGGCGGGGGTCGTT |
| | | GGGCGGTCAGCCAGGCG |
| | | GGCCATTTACCGTAAGT |
| | | TATGTAACGCGGAACTC |
| | | CATATATGGGCTATGAA |
| | | CTAATGACCCCGTAATT |
| | | GATTACTATTAATAACT |
| | | AGTCAATAATCAATGTC |
| | | AACGCGTATGGTACCTG |
| | | CGGAGGATGCCGAGGAT |
| | | AACCTTGTTACTAGCCTC |
| | | CGCCTGGCCGTTGGACT |
| | | GTGGATAATATGGCGTA |
| | | GAGGATCCTCTGCGCGC |
| | | TCGCTCGCTCACTGAGG |
| | | CCGCCCGGGCAAAGCCC |
| | | GGGCGTCGGGCGACCTT |
| | | TGGTCGCCCGGCCTCAG |
| | | TGAGCGAGCGAGCGCGC |
| | | AGAGAA |
| 53 | N-term NpuDnaE intein | CLSYETEILTVEYGLLPIG |
| | fragment | KIVEKRIECTVYSVDNNG |
| | | NIYTQPVAQWHDRGEQE |
| | | VFEYCLEDGSLIRATKDH |
| | | KFMTVDGQMLPIDEIFER |
| | | ELDLMRVDNLPN |
| 54 | C-term NpuDnaE intein | MIKIATRKYLGKQNVYDI |
| | fragment | GVERDHNFALKNGFIASN |

What is claimed is:

1. A composition comprising:
a) a first exogenous nucleic acid construct comprising:
i) a first polynucleotide of interest; and
ii) a sequence encoding an N-terminal fragment of a functional selectable protein fused in-frame to a sequence encoding an N-terminal fragment of an intein; and
b) a second exogenous nucleic acid construct comprising:
i) a second polynucleotide of interest; and
ii) a sequence encoding a C-terminal fragment of the intein fused in-frame to a sequence encoding a C-terminal fragment of the functional selectable protein;
wherein, when expressed, the N-terminal fragment of the functional selectable protein and the C-terminal fragment of the selectable protein are joined together to form the functional selectable protein by the N-terminal fragment of the intein and the C-terminal fragment of the intein,
wherein the functional selectable protein is selected from the group consisting of glutamine synthetase (GS), phenylalanine hydroxylase (PAH), dihydrofolate reductase (DHFR), and thymidylate synthase (TYMS), and
wherein the first and second nucleic acid constructs are exogenous to an eukaryotic host cell in which they are introduced.

2. The composition of claim 1, wherein the first exogenous nucleic acid construct is a first plasmid and the second nucleic acid construct is a second plasmid.

3. The composition of claim 1 or 2, wherein the first polynucleotide of interest encodes an adeno-associated virus (AAV) Rep protein, an AAV Cap protein, an adenoviral helper protein, a first payload, or any combination thereof and/or wherein the second polynucleotide of interest encodes an adeno-associated virus (AAV) Rep protein, an AAV Cap protein, an adenoviral helper protein, a second payload, or any combination thereof.

4. The composition of any one of claims 1-3, wherein the second exogenous nucleic acid construct comprises:
a first promoter and the second polynucleotide of interest, wherein the first promoter is operably linked to the second polynucleotide of interest;
a second promoter and the sequence encoding the C-terminal fragment of the intein fused in-frame to the sequence encoding the C-terminal fragment of the functional selectable protein, wherein the second promoter is operably linked to the sequence encoding the C-terminal fragments of the intein and the functional selectable protein, and
wherein the 3′ end of the coding strand of the second polynucleotide of interest is adjacent to the 3′ end of the coding strand for the C-terminal fragment of the intein fused in-frame to the sequence encoding the C-terminal fragment of the functional selectable protein such that a direction of transcription of the second polynucleotide of interest and a direction of transcription of the C-terminal fragment of the intein fused in-frame to the sequence encoding a C-terminal fragment of the functional selectable protein are towards each other.

5. The composition of any one of claims 1-4, wherein the first exogenous nucleic acid construct comprises:
a first promoter and the first polynucleotide of interest, wherein the first promoter is operably linked to the first polynucleotide of interest;
a second promoter and the sequence encoding the N-terminal fragment of the functional selectable protein fused in-frame to the sequence encoding the N-terminal fragment of the intein, wherein the second promoter is operably linked to the sequence encoding the N-terminal fragments of the functional selectable protein and the intein,
wherein the 5′ end of the coding strand for the first polynucleotide of interest is adjacent to the 5′ end of the coding strand for the N-terminal fragment of the functional selectable protein and the N-terminal fragment of the intein such that a direction of transcription of the first polynucleotide of interest and a direction of transcription of the N-terminal fragment of the functional selectable protein and the N-terminal fragment of the intein proceeds away from the 5′ end of the respective sequences.

6. The composition of claim 4 or 5, wherein the second polynucleotide of interest encodes for an AAV Rep and/or an AAV Cap protein and the first polynucleotide of interest encodes a first payload.

7. The composition of any one of claims 3-6, wherein the first and/or second payload is a guide RNA, a tRNA, a gene, a transgene, encodes a protein, comprises a gene for replacement gene therapy, or comprises a homology construct for homologous recombination.

8. The composition of any one of claims 1-7, wherein the N-terminal residue of the C-terminal fragment of the functional selectable protein is a cysteine or serine and wherein the N-terminal fragment and the C-terminal fragment are spliced together at a split point in the functional selectable protein, wherein the split point is immediately N-terminus to the cysteine or serine within a catalytic domain of the functional selectable protein.

9. The composition of claim 8, wherein the N-terminal residue of the C-terminal fragment of the functional selectable protein is cysteine.

10. The composition of any one of claims 1-9, wherein the intein is derived from the Nostoc punctiforme (Npu) DnaE intein, the Synechocystis species, strain PCC6803 (Ssp) DnaE intein, or the consensus DnaE intein (Cfa), and optionally wherein the N-terminal fragment of the intein comprises the amino acid sequence of SEQ ID NO:53 and/or wherein the C-terminal fragment of the intein comprises the amino acid sequence of SEQ ID NO:54.

11. The composition of any one of claims 1-10, wherein the first exogenous nucleic acid construct or second exogenous nucleic acid construct further encodes a helper enzyme that facilitates production of a molecule required for growth of a host cell into which the first exogenous nucleic acid construct and the second exogenous nucleic acid construct are introduced, wherein the molecule is produced by enzymatic activity of the functional selectable marker.

12. The composition of any one of claims 1-11, wherein expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein is suppressed.

13. The composition of claim 12, wherein the expression from the sequence encoding the N-terminal fragment of the functional selectable protein fused in-frame to the sequence encoding the N-terminal fragment of the intein and/or the C-terminal fragment of the intein fused in-frame to the sequence encoding the C-terminal fragment of the functional selectable protein is suppressed via an attenuated promoter.

14. The composition of claim 13, wherein the attenuated promoter comprises an attenuated EF1alpha promoter; optionally, wherein the attenuated EF1alpha promoter has a sequence that is SEQ ID NO: 43.

15. The composition of claim 12, wherein the expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein is suppressed via the functional selectable protein comprising a mutation.

16. The composition of claim 15, wherein the functional selectable protein is GS, and the mutation is R324C, R324S, or R341C.

17. The composition of any one of claims 11-15, wherein the functional selectable protein is phenylalanine hydroxylase (PAH) and the helper enzyme is GTP cyclohydrolase I (GTP-CH1).

18. The composition of any one of claims 1-14, wherein the functional selectable protein is PAH, and wherein
(i) the N-terminal fragment of the PAH comprises amino acids 1-236 of SEQ ID NO:1 and the C-terminal fragment of the PAH comprises amino acids 237-452 of SEQ ID NO:1;
(ii) the N-terminal fragment of the PAH comprises amino acids 1-264 of SEQ ID NO:1 and the C-terminal fragment of the PAH comprises amino acids 265-452 of SEQ ID NO:1;
(iii) the N-terminal fragment of the PAH comprises amino acids 1-283 of SEQ ID NO:1 and the C-terminal fragment of the PAH comprises amino acids 284-452 of SEQ ID NO:1; or
(iv) the N-terminal fragment of the PAH comprises amino acids 1-333 of SEQ ID NO:1 and the C-terminal fragment of the PAH comprises amino acids 334-452 of SEQ ID NO:1.

19. The composition of claim 18, wherein the N-terminal fragment of PAH is fused to the N-terminal fragment of the intein and the C-terminal fragment of the intein is fused to the C-terminal fragment of the PAH, and the N-terminal fragment of the intein and the C-terminal fragment of the intein are capable of being spliced out to generate a functional PAH comprising the amino acid sequence of SEQ ID NO:1.

20. The composition of any one of claims 1-14, wherein the functional selectable protein is GS and wherein
(i) the N-terminal fragment of the GS comprises amino acids 1-52 of SEQ ID NO:23 and the C-terminal fragment of the GS comprises amino acids 53-373 of SEQ ID NO:23;
(ii) the N-terminal fragment of the GS comprises amino acids 1-116 of SEQ ID NO:23 and the C-terminal fragment of the GS comprises amino acids 117-373 of SEQ ID NO:23;
(iii) the N-terminal fragment of the GS comprises amino acids 1-182 of SEQ ID NO:23 and the C-terminal fragment of the GS comprises amino acids 183-373 of SEQ ID NO:23;
(iv) the N-terminal fragment of the GS comprises amino acids 1-228 of SEQ ID NO:23 and the C-terminal fragment of the GS comprises amino acids 229-373 of SEQ ID NO:23; or
(v) the N-terminal fragment of the GS comprises amino acids 1-251 of SEQ ID NO:23 and the C-terminal fragment of the GS comprises amino acids 252-373 of SEQ ID NO:23.

21. The composition of claim 20, wherein N-terminal fragment of GS is fused in-frame to the sequence encoding the N-terminal fragment of the intein and the C-terminal fragment of the intein fused in-frame to the sequence encoding the C-terminal fragment of GS, and the N-terminal fragment of the intein and the C-terminal fragment of the intein are capable of being spliced out to generate a functional GS comprising the amino acid sequence of SEQ ID NO:23.

22. The composition of any one of claims 1-14, wherein functional selectable protein is TYMS and wherein
(i) the N-terminal fragment of the TYMS comprises amino acids 1-40 of SEQ ID NO:34 and the C-terminal fragment of the TYMS comprises amino acids 41-279 of SEQ ID NO:34;
(ii) the N-terminal fragment of the TYMS comprises amino acids 1-160 of SEQ ID NO:34 and the C-terminal fragment of the TYMS comprises amino acids 161-279 of SEQ ID NO:34;
(iii) the N-terminal fragment of the TYMS comprises amino acids 1-164 of SEQ ID NO:34 and the C-terminal fragment of the TYMS comprises amino acids 165-279 of SEQ ID NO:34; or
(iv) the N-terminal fragment of the TYMS comprises amino acids 1-175 of SEQ ID NO:34 and the C-terminal fragment of the TYMS comprises amino acids 176-279 of SEQ ID NO:34.

23. The composition of claim 22, wherein N-terminal fragment of TYMS is fused in-frame to the sequence encoding the N-terminal fragment of the intein and the C-terminal fragment of the intein fused in-frame to the sequence encoding the C-terminal fragment of TYMS, and the N-terminal fragment of the intein and the C-terminal fragment of the intein are capable of being spliced out to generate a functional TYMS comprising the amino acid sequence of SEQ ID NO:34.

24. A method of generating a recombinant eukaryotic host cell that can be selected to retain a first exogenous nucleic acid construct and a second exogenous nucleic acid construct with a single selective pressure, the method comprising:
introducing into a eukaryotic host cell the first exogenous nucleic acid construct and the second exogenous nucleic acid construct according to any one of claims 1-23,
wherein upon application of the single selective pressure, the eukaryotic host cell comprising the first exogenous nucleic acid construct and the second exogenous nucleic acid construct is selected.

25. The method of claim 24, wherein the host cell is a mammalian cell and optionally wherein the mammalian cell is a human embryonic kidney (HEK) cell, Chinese hamster ovary (CHO) cell, or HeLa cell, and optionally wherein the host cell is suspension-adapted.

26. The method of claim 24 or 25, wherein the functional selectable protein is phenylalanine hydroxylase (PAH) and the single selective pressure is a culture medium deficient in tyrosine, wherein the host cell does not grow in the culture medium in absence the first exogenous nucleic acid construct and the second exogenous nucleic acid construct.

27. The method of claim 26, wherein the culture medium comprises phenylalanine and (6R)-5,6,7,8-tetrahydrobiopterin (BH4) or a BH4 precursor, optionally wherein the BH4 precursor is 7,8-dihydrobiopterin (7,8-BH2).

28. The method of claim 26 or 27, wherein the host cell expresses GTP cyclohydrolase I (GTP-CH1).

29. The method of claim 24 or 25, wherein the functional selectable protein is GS and the single selective pressure comprises a culture medium deficient in glutamine, wherein the host cell does not grow in the culture medium in absence of the first exogenous nucleic acid construct and the second exogenous nucleic acid construct.

30. The method of claim 24 or 25, wherein the functional selectable protein is TYMS and the single selective pressure comprises a culture medium deficient in hypoxanthine and/or thymidine.

31. The method of any one of claims 24-30, wherein expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein is suppressed and upon application of the single selective pressure to the eukaryotic host cell, the single selective pressure selects for the eukaryotic host cell comprising a high copy number of the first exogenous nucleic acid construct and the second exogenous nucleic acid construct.

32. The method of claim 31, wherein the expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein is suppressed via administration of an inhibitor of the functional selectable protein.

33. The method of claim 32, wherein the functional selectable protein is GS, and the inhibitor is Methionine Sulfoximine (MSX).

34. The method of any one of claims 24-33, further comprising applying the single selective pressure to the eukaryotic host cell by culturing the eukaryotic host cell in a culture medium deficient in at least one molecule required for growth of the eukaryotic host cell.

35. The method of claim 34, further comprising applying a second selective pressure, wherein application of the second selective pressure selects for high expression of the N-terminal fragment of the functional selectable protein fused in-frame to the sequence encoding the N-terminal fragment of the intein and the C-terminal fragment of the intein fused in-frame to the sequence encoding the C-terminal fragment of the functional selectable protein in the eukaryotic host cell.

36. The method of claim 35, wherein the second selective pressure is the presence of an inhibitor of the functional selectable protein; optionally, wherein the functional selectable protein is GS and the inhibitor is MSX.

37. A method for selecting a recombinant eukaryotic host cell that has retained the first exogenous nucleic acid construct and the second exogenous nucleic acid construct according to any one of claims 1-23, the method comprising:
applying a single selective pressure to an eukaryotic host cell into which the first exogenous nucleic acid construct and the second exogenous nucleic acid construct has been introduced,
wherein upon application of the single selective pressure, the eukaryotic host cell comprising the first exogenous nucleic acid construct and the second exogenous nucleic acid construct is selected.

38. The method of claim 37, wherein applying the single selective pressure to the eukaryotic host cell comprises culturing the eukaryotic host cell in a culture medium deficient in at least one molecule required for growth of the eukaryotic host cell, wherein the culturing is for a period of time sufficient for selection of the recombinant eukaryotic host cell that has retained the first exogenous nucleic acid construct and the second exogenous nucleic acid construct.

39. The method of claim 36 or 37, wherein the host cell is a mammalian cell and optionally wherein the mammalian cell is a human embryonic kidney (HEK) cell, Chinese hamster ovary (CHO) cell, or HeLa cell, and optionally wherein the host cell is suspension-adapted.

40. The method of any one of claims 36-39, wherein the functional selectable protein is phenylalanine hydroxylase (PAH) and the single selective pressure is a culture medium deficient in tyrosine, wherein the host cell does not grow in the culture medium in absence the first exogenous nucleic acid construct and the second exogenous nucleic acid construct.

41. The method of claim 40, wherein the culture medium comprises phenylalanine and (6R)-5,6,7,8-tetrahydrobiopterin (BH4) or a BH4 precursor, optionally wherein the BH4 precursor is 7,8-dihydrobiopterin (7,8-BH2).

42. The method of claim 40 or 41, wherein the host cell expresses GTP cyclohydrolase I (GTP-CH1).

43. The method of any one of claims 36-39, wherein the functional selectable protein is GS and the single selective pressure comprises a culture medium deficient in glutamine, wherein the host cell does not grow in the culture medium in absence of the first exogenous nucleic acid construct and the second exogenous nucleic acid construct.

44. The method of any one of claims 36-39, wherein the functional selectable protein is TYMS and the single selective pressure comprises a culture medium deficient in hypoxanthine and/or thymidine.

45. The method of any one of claims 36-44, wherein expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein is suppressed and upon application of the single selective pressure to the eukaryotic host cell, the single selective pressure selects for the eukaryotic host cell comprising a high copy number of the first exogenous nucleic acid construct and the second exogenous nucleic acid construct.

46. The method of claim 45, wherein the method comprises administering to the host cell an inhibitor of the functional selectable protein for suppressing the expression from the sequence encoding the N-terminal fragment of the functional selectable protein fused in-frame to the sequence encoding the N-terminal fragment of an intein and/or the C-terminal fragment of the intein fused in-frame to the sequence encoding the C-terminal fragment of the functional selectable protein, optionally, wherein the method comprises culturing the host cell in a culture medium comprising the inhibitor simultaneously with or subsequently to culturing the host cell in a culture medium deficient in at least one molecule required for growth of the host cell.

47. The method of claim 46, wherein the functional selectable protein is GS, and the inhibitor is Methionine Sulfoximine (MSX).

48. The method of any one of claims 37-47, wherein the method comprises introducing the first and second exogenous nucleic acid constructs into the eukaryotic host cell.

49. A recombinant eukaryotic host cell or cell line, wherein the recombinant eukaryotic host cell or cell line is selected to retain the first exogenous nucleic acid construct and the second exogenous nucleic acid construct as set forth in any one of claims 1-23, with a single selective pressure.

50. A eukaryotic host cell or cell line selected to retain the first exogenous nucleic acid construct and the second exogenous nucleic acid construct by a method as set forth in any one of claims 24-48.

51. A method for producing a plurality of recombinant adeno-associated virus (rAAV) virions, the method comprising:
culturing the recombinant eukaryotic host cell or cell line as set forth in claim 49 or 50 in a culture medium to produce the rAAV.

52. The method of claim 51, wherein
the first polynucleotide of interest encodes a first payload,
the second polynucleotide of interest encodes AAV Rep proteins and AAV Cap proteins, and
the functional selectable protein is a first functional selectable protein,
and the cell further comprises a nucleic acid construct comprising a polynucleotide sequence encoding a second functional selectable protein and one or more of AAV helper proteins and/or one or more VA RNA.

53. The method of claim 51, wherein
the first polynucleotide of interest encodes AAV Rep proteins and AAV Cap proteins,
the second polynucleotide of interest encodes a first payload, and
the functional selectable protein is a first functional selectable protein,
and the cell further comprises a nucleic acid construct comprising a polynucleotide sequence encoding a second functional selectable protein and one or more of AAV helper proteins and/or one or more VA RNA.

54. The method of claim 51, wherein
the first polynucleotide of interest encodes AAV Rep proteins and AAV Cap proteins,
the second polynucleotide of interest encodes one or more of AAV helper proteins and/or one or more VA RNA, and
the functional selectable protein is a first functional selectable protein,
and the cell further comprises a nucleic acid construct comprising a polynucleotide sequence encoding a second functional selectable protein and a first payload.

55. The method of claim 51, wherein
the first polynucleotide of interest encodes a first payload,
the second polynucleotide of interest encodes one or more of AAV helper proteins and/or one or more VA RNA, and
the functional selectable protein is a first functional selectable protein,
and the cell further comprises a nucleic acid construct comprising a polynucleotide sequence encoding a second functional selectable protein and AAV Rep proteins and AAV Cap proteins.

56. The method of any one of claims 51-55, wherein the AAV Rep proteins comprise one or more of Rep78, Rep68, Rep52, Rep40, or any combination thereof.

57. The method of any one of claims 51-56, wherein the AAV Cap proteins one or more of VP1, VP2, VP3, or any combination thereof.

58. The method of any one of claims 51-56, wherein the AAV helper proteins comprise one or more of E1A, E1B, E2A, E4, or any combination thereof.

59. The method of any one of claims 51-58, wherein the sequence encoding VA RNA encodes for a mutant VA RNA; optionally, wherein the mutant VA RNA comprises a G16A mutation, a G60A mutation, or a combination thereof.

60. The method of any one of claims 51-59, wherein the culturing comprises culturing the recombinant eukaryotic host cell or cell line in a culture medium deficient in a molecule required for growth of the recombinant eukaryotic host cell or cell line.

61. The method of any one of claims 51-60, wherein the expression of one or more of an AAV Rep, an AAV Cap protein, an adenoviral helper protein, and a first payload is inducible.

62. The method of any one of claims 51-61, wherein the first functional selectable protein is as set forth in any one of claims 1-23 and the second functional selectable protein is different from the first functional selectable protein.
